SEQUENCE LISTING (101 sequences)

SEQ ID NO: 1 - protein, 518 aa, Artificial Sequence
  Synthetic Construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGER

SEQ ID NO: 2 - protein, 611 aa, Artificial Sequence
  Synthetic Construct
RPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 3 - protein, 658 aa, Artificial Sequence
  Synthetic Construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRC

SEQ ID NO: 4 - protein, 471 aa, Artificial Sequence
  Synthetic Construct
GSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 5 - protein, 783 aa, Artificial Sequence
  Synthetic Construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQV

SEQ ID NO: 6 - protein, 346 aa, Artificial Sequence
  Synthetic Construct
IRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 7 - RNA, 89 nt, Artificial Sequence
  Synthetic Construct
  misc_feature 89: A, G, C, or U
  misc_feature 89: Can be present in repeats of any integer between 18 and 35
gucgucuauaggacggcgaguuuuucaacgggugugccaauggccacuuuccagguggcaaagcccguugagcuucaagaaguggcacn

SEQ ID NO: 8 - RNA, 64 nt, Artificial Sequence
  Synthetic Construct
  misc_feature 64: A, G, C, or U
  misc_feature 64: Can be present in repeats of any integer between 18 and 35
caacgggugugccaauggccacuuuccagguggcaaagcccguugagcuucaagaaguggcacn

SEQ ID NO: 9 - RNA, 73 nt, Artificial Sequence
  Synthetic Construct
  misc_feature 73: A, G, C, or U
  misc_feature 73: Can be present in repeats of any integer between 18 and 35
gucgucuauaggacggcgaguuuuugugccaauggccacuuuccagguggcaaaagcuucaagaaguggcacn

SEQ ID NO: 10 - RNA, 62 nt, Artificial Sequence
  Synthetic Construct
  misc_feature 62: A, G, C, or U
  misc_feature 62: Can be present in repeats of any integer between 18 and 35
gucgucuauaggacggcgaguuuuucaacgggugugcccguugagcuucaagaaguggcacn

SEQ ID NO: 11 - protein, 644 aa, Artificial Sequence
  Synthetic Construct
PKKKRKVPGMAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERSRGGSGSSGGSGGSGGSGEMWHEGLEEASRLYFGERNVKGMFEVLEPLHAMMERGPQTLKETSFNQAYGRDLMEAQEWCRKYMKSGNVKDLTQAWDLYYHVFRRISKQASPKKKRKV

SEQ ID NO: 12 - protein, 752 aa, Artificial Sequence
  Synthetic Construct
PKKKRKVPGMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSGGGSGGGLKRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDIASPKKKRKV

SEQ ID NO: 13 - protein, 784 aa, Artificial Sequence
  Synthetic Construct
PKKKRKVPGMAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCSRGGSGSSGGSGGSGGSGEMWHEGLEEASRLYFGERNVKGMFEVLEPLHAMMERGPQTLKETSFNQAYGRDLMEAQEWCRKYMKSGNVKDLTQAWDLYYHVFRRISKQASPKKKRKV

SEQ ID NO: 14 - protein, 612 aa, Artificial Sequence
  Synthetic Construct
PKKKRKVPGMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSGGGSGGGLKGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDIASPKKKRKV

SEQ ID NO: 15 - protein, 909 aa, Artificial Sequence
  Synthetic Construct
PKKKRKVPGMAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVSRGGSGSSGGSGGSGGSGEMWHEGLEEASRLYFGERNVKGMFEVLEPLHAMMERGPQTLKETSFNQAYGRDLMEAQEWCRKYMKSGNVKDLTQAWDLYYHVFRRISKQASPKKKRKV

SEQ ID NO: 16 - protein, 487 aa, Artificial Sequence
  Synthetic Construct
PKKKRKVPGMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSGGGSGGGLKIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDIASPKKKRKV

SEQ ID NO: 17 - DNA, 1932 bp, Artificial Sequence
  Synthetic Construct
cccaagaagaagcgcaaggtgcccgggatggccgtgaagagcatgaaggtgaagctgcgcctggacaacatgcccgagatccgcgccggcctgtggaagctgcacaccgaggtgaacgccggcgtgcgctactacaccgagtggctgagcctgctgcgccaggagaacctgtaccgccgcagccccaacggcgacggcgagcaggagtgctacaagaccgccgaggagtgcaaggccgagctgctggagcgcctgcgcgcccgccaggtggagaacggccactgcggccccgccggcagcgacgacgagctgctgcagctggcccgccagctgtacgagctgctggtgccccaggccatcggcgccaagggcgacgcccagcagatcgcccgcaagttcctgagccccctggccgacaaggacgccgtgggcggcctgggcatcgccaaggccggcaacaagccccgctgggtgcgcatgcgcgaggccggcgagcccggctgggaggaggagaaggccaaggccgaggcccgcaagagcaccgaccgcaccgccgacgtgctgcgcgccctggccgacttcggcctgaagcccctgatgcgcgtgtacaccgacagcgacatgagcagcgtgcagtggaagcccctgcgcaagggccaggccgtgcgcacctgggaccgcgacatgttccagcaggccatcgagcgcatgatgagctgggagagctggaaccagcgcgtgggcgaggcctacgccaagctggtggagcagaagagccgcttcgagcagaagaacttcgtgggccaggagcacctggtgcagctggtgaaccagctgcagcaggacatgaaggaggccagccacggcctggagagcaaggagcagaccgcccactacctgaccggccgcgccctgcgcggcagcgacaaggtgttcgagaagtgggagaagctggaccccgacgcccccttcgacctgtacgacaccgagatcaagaacgtgcagcgccgcaacacccgccgcttcggcagccacgacctgttcgccaagctggccgagcccaagtaccaggccctgtggcgcgaggacgccagcttcctgacccgctacgccgtgtacaacagcatcgtgcgcaagctgaaccacgccaagatgttcgccaccttcaccctgcccgacgccaccgcccaccccatctggacccgcttcgacaagctgggcggcaacctgcaccagtacaccttcctgttcaacgagttcggcgagggccgccacgccatccgcttccagaagctgctgaccgtggaggacggcgtggccaaggaggtggacgacgtgaccgtgcccatcagcatgagcgcccagctggacgacctgctgccccgcgacccccacgagctggtggccctgtacttccaggactacggcgccgagcagcacctggccggcgagttcggcggcgccaagatccagtaccgccgcgaccagctgaaccacctgcacgcccgccgcggcgcccgcgacgtgtacctgaacctgagcgtgcgcgtgcagagccagagcgaggcccgcggcgagcgctctagaggcggcagcggcagcagcggcggcagcggcggcagcggcggcagcggcgagatgtggcacgagggcctggaggaggccagccgcctgtacttcggcgagcgcaacgtgaagggcatgttcgaggtgctggagcccctgcacgccatgatggagcgcggcccccagaccctgaaggagaccagcttcaaccaggcctacggccgcgacctgatggaggcccaggagtggtgccgcaagtacatgaagagcggcaacgtgaaggacctgacccaggcctgggacctgtactaccacgtgttccgccgcatcagcaagcaggctagccccaagaagaagcgcaaggtg

SEQ ID NO: 18 - DNA, 2256 bp, Artificial Sequence
  Synthetic Construct
cccaagaagaagcgcaaggtgcccgggatgggcgtgcaggtggagaccatcagccccggcgacggccgcaccttccccaagcgcggccagacctgcgtggtgcactacaccggcatgctggaggacggcaagaagttcgacagcagccgcgaccgcaacaagcccttcaagttcatgctgggcaagcaggaggtgatccgcggctgggaggagggcgtggcccagatgagcgtgggccagcgcgccaagctgaccatcagccccgactacgcctacggcgccaccggccaccccggcatcatccccccccacgccaccctggtgttcgacgtggagctgctgaagctggagggcggcagcggcggcagcggcggcggcagcggcggcggccttaagcgccccccctacgccgccgtgttccgcctggtgggcgacaaccaccgcgccttcgtgcacttcgacaagctgagcgactacctggccgagcaccccgacgacggcaagctgggcagcgagggcctgctgagcggcctgcgcgtgatgagcgtggacctgggcctgcgcaccagcgccagcatcagcgtgttccgcgtggcccgcaaggacgagctgaagcccaacagcgagggccgcgtgcccttctgcttccccatcgagggcaacgagaacctggtggccgtgcacgagcgcagccagctgctgaagctgcccggcgagaccgagagcaaggacctgcgcgccatccgcgaggagcgccagcgcaccctgcgccagctgcgcacccagctggcctacctgcgcctgctggtgcgctgcggcagcgaggacgtgggccgccgcgagcgcagctgggccaagctgatcgagcagcccatggacgccaaccagatgacccccgactggcgcgaggccttcgaggacgagctgcagaagctgaagagcctgtacggcatctgcggcgaccgcgagtggaccgaggccgtgtacgagagcgtgcgccgcgtgtggcgccacatgggcaagcaggtgcgcgactggcgcaaggacgtgcgcagcggcgagcgccccaagatccgcggctaccagaaggacgtggtgggcggcaacagcatcgagcagatcgagtacctggagcgccagtacaagttcctgaagagctggagcttcttcggcaaggtgagcggccaggtgatccgcgccgagaagggcagccgcttcgccatcaccctgcgcgagcacatcgaccacgccaaggaggaccgcctgaagaagctggccgaccgcatcatcatggaggccctgggctacgtgtacgccctggacgacgagcgcggcaagggcaagtgggtggccaagtaccccccctgccagctgatcctgctggaggagctgagcgagtaccagttcaacaacgaccgcccccccagcgagaacaaccagctgatgcagtggagccaccgcggcgtgttccaggagctgctgaaccaggcccaggtgcacgacctgctggtgggcaccatgtacgccgccttcagcagccgcttcgacgcccgcaccggcgcccccggcatccgctgccgccgcgtgcccgcccgctgcgcccgcgagcagaaccccgagcccttcccctggtggctgaacaagttcgtggccgagcacaagctggacggctgccccctgcgcgccgacgacctgatccccaccggcgagggcgagttcttcgtgagccccttcagcgccgaggagggcgacttccaccagatccacgccgacctgaacgccgcccagaacctgcagcgccgcctgtggagcgacttcgacatcagccagatccgcctgcgctgcgactggggcgaggtggacggcgagcccgtgctgatcccccgcaccaccggcaagcgcaccgccgacagctacggcaacaaggtgttctacaccaagaccggcgtgacctactacgagcgcgagcgcggcaagaagcgccgcaaggtgttcgcccaggaggagctgagcgaggaggaggccgagctgctggtggaggccgacgaggcccgcgagaagagcgtggtgctgatgcgcgaccccagcggcatcatcaaccgcggcgactggacccgccagaaggagttctggagcatggtgaaccagcgcatcgagggctacctggtgaagcagatccgcagccgcgtgcgcctgcaggagagcgcctgcgagaacaccggcgacatcgctagccccaagaagaagcgcaaggtg

SEQ ID NO: 19 - DNA, 2352 bp, Artificial Sequence
  Synthetic Construct
cccaagaagaagcgcaaggtgcccgggatggccgtgaagagcatgaaggtgaagctgcgcctggacaacatgcccgagatccgcgccggcctgtggaagctgcacaccgaggtgaacgccggcgtgcgctactacaccgagtggctgagcctgctgcgccaggagaacctgtaccgccgcagccccaacggcgacggcgagcaggagtgctacaagaccgccgaggagtgcaaggccgagctgctggagcgcctgcgcgcccgccaggtggagaacggccactgcggccccgccggcagcgacgacgagctgctgcagctggcccgccagctgtacgagctgctggtgccccaggccatcggcgccaagggcgacgcccagcagatcgcccgcaagttcctgagccccctggccgacaaggacgccgtgggcggcctgggcatcgccaaggccggcaacaagccccgctgggtgcgcatgcgcgaggccggcgagcccggctgggaggaggagaaggccaaggccgaggcccgcaagagcaccgaccgcaccgccgacgtgctgcgcgccctggccgacttcggcctgaagcccctgatgcgcgtgtacaccgacagcgacatgagcagcgtgcagtggaagcccctgcgcaagggccaggccgtgcgcacctgggaccgcgacatgttccagcaggccatcgagcgcatgatgagctgggagagctggaaccagcgcgtgggcgaggcctacgccaagctggtggagcagaagagccgcttcgagcagaagaacttcgtgggccaggagcacctggtgcagctggtgaaccagctgcagcaggacatgaaggaggccagccacggcctggagagcaaggagcagaccgcccactacctgaccggccgcgccctgcgcggcagcgacaaggtgttcgagaagtgggagaagctggaccccgacgcccccttcgacctgtacgacaccgagatcaagaacgtgcagcgccgcaacacccgccgcttcggcagccacgacctgttcgccaagctggccgagcccaagtaccaggccctgtggcgcgaggacgccagcttcctgacccgctacgccgtgtacaacagcatcgtgcgcaagctgaaccacgccaagatgttcgccaccttcaccctgcccgacgccaccgcccaccccatctggacccgcttcgacaagctgggcggcaacctgcaccagtacaccttcctgttcaacgagttcggcgagggccgccacgccatccgcttccagaagctgctgaccgtggaggacggcgtggccaaggaggtggacgacgtgaccgtgcccatcagcatgagcgcccagctggacgacctgctgccccgcgacccccacgagctggtggccctgtacttccaggactacggcgccgagcagcacctggccggcgagttcggcggcgccaagatccagtaccgccgcgaccagctgaaccacctgcacgcccgccgcggcgcccgcgacgtgtacctgaacctgagcgtgcgcgtgcagagccagagcgaggcccgcggcgagcgccgccccccctacgccgccgtgttccgcctggtgggcgacaaccaccgcgccttcgtgcacttcgacaagctgagcgactacctggccgagcaccccgacgacggcaagctgggcagcgagggcctgctgagcggcctgcgcgtgatgagcgtggacctgggcctgcgcaccagcgccagcatcagcgtgttccgcgtggcccgcaaggacgagctgaagcccaacagcgagggccgcgtgcccttctgcttccccatcgagggcaacgagaacctggtggccgtgcacgagcgcagccagctgctgaagctgcccggcgagaccgagagcaaggacctgcgcgccatccgcgaggagcgccagcgcaccctgcgccagctgcgcacccagctggcctacctgcgcctgctggtgcgctgctctagaggcggcagcggcagcagcggcggcagcggcggcagcggcggcagcggcgagatgtggcacgagggcctggaggaggccagccgcctgtacttcggcgagcgcaacgtgaagggcatgttcgaggtgctggagcccctgcacgccatgatggagcgcggcccccagaccctgaaggagaccagcttcaaccaggcctacggccgcgacctgatggaggcccaggagtggtgccgcaagtacatgaagagcggcaacgtgaaggacctgacccaggcctgggacctgtactaccacgtgttccgccgcatcagcaagcaggctagccccaagaagaagcgcaaggtg

SEQ ID NO: 20 - DNA, 1836 bp, Artificial Sequence
  Synthetic Construct
cccaagaagaagcgcaaggtgcccgggatgggcgtgcaggtggagaccatcagccccggcgacggccgcaccttccccaagcgcggccagacctgcgtggtgcactacaccggcatgctggaggacggcaagaagttcgacagcagccgcgaccgcaacaagcccttcaagttcatgctgggcaagcaggaggtgatccgcggctgggaggagggcgtggcccagatgagcgtgggccagcgcgccaagctgaccatcagccccgactacgcctacggcgccaccggccaccccggcatcatccccccccacgccaccctggtgttcgacgtggagctgctgaagctggagggcggcagcggcggcagcggcggcggcagcggcggcggccttaagggcagcgaggacgtgggccgccgcgagcgcagctgggccaagctgatcgagcagcccatggacgccaaccagatgacccccgactggcgcgaggccttcgaggacgagctgcagaagctgaagagcctgtacggcatctgcggcgaccgcgagtggaccgaggccgtgtacgagagcgtgcgccgcgtgtggcgccacatgggcaagcaggtgcgcgactggcgcaaggacgtgcgcagcggcgagcgccccaagatccgcggctaccagaaggacgtggtgggcggcaacagcatcgagcagatcgagtacctggagcgccagtacaagttcctgaagagctggagcttcttcggcaaggtgagcggccaggtgatccgcgccgagaagggcagccgcttcgccatcaccctgcgcgagcacatcgaccacgccaaggaggaccgcctgaagaagctggccgaccgcatcatcatggaggccctgggctacgtgtacgccctggacgacgagcgcggcaagggcaagtgggtggccaagtaccccccctgccagctgatcctgctggaggagctgagcgagtaccagttcaacaacgaccgcccccccagcgagaacaaccagctgatgcagtggagccaccgcggcgtgttccaggagctgctgaaccaggcccaggtgcacgacctgctggtgggcaccatgtacgccgccttcagcagccgcttcgacgcccgcaccggcgcccccggcatccgctgccgccgcgtgcccgcccgctgcgcccgcgagcagaaccccgagcccttcccctggtggctgaacaagttcgtggccgagcacaagctggacggctgccccctgcgcgccgacgacctgatccccaccggcgagggcgagttcttcgtgagccccttcagcgccgaggagggcgacttccaccagatccacgccgacctgaacgccgcccagaacctgcagcgccgcctgtggagcgacttcgacatcagccagatccgcctgcgctgcgactggggcgaggtggacggcgagcccgtgctgatcccccgcaccaccggcaagcgcaccgccgacagctacggcaacaaggtgttctacaccaagaccggcgtgacctactacgagcgcgagcgcggcaagaagcgccgcaaggtgttcgcccaggaggagctgagcgaggaggaggccgagctgctggtggaggccgacgaggcccgcgagaagagcgtggtgctgatgcgcgaccccagcggcatcatcaaccgcggcgactggacccgccagaaggagttctggagcatggtgaaccagcgcatcgagggctacctggtgaagcagatccgcagccgcgtgcgcctgcaggagagcgcctgcgagaacaccggcgacatcgctagccccaagaagaagcgcaaggtg

SEQ ID NO: 21 - DNA, 2727 bp, Artificial Sequence
  Synthetic Construct
cccaagaagaagcgcaaggtgcccgggatggccgtgaagagcatgaaggtgaagctgcgcctggacaacatgcccgagatccgcgccggcctgtggaagctgcacaccgaggtgaacgccggcgtgcgctactacaccgagtggctgagcctgctgcgccaggagaacctgtaccgccgcagccccaacggcgacggcgagcaggagtgctacaagaccgccgaggagtgcaaggccgagctgctggagcgcctgcgcgcccgccaggtggagaacggccactgcggccccgccggcagcgacgacgagctgctgcagctggcccgccagctgtacgagctgctggtgccccaggccatcggcgccaagggcgacgcccagcagatcgcccgcaagttcctgagccccctggccgacaaggacgccgtgggcggcctgggcatcgccaaggccggcaacaagccccgctgggtgcgcatgcgcgaggccggcgagcccggctgggaggaggagaaggccaaggccgaggcccgcaagagcaccgaccgcaccgccgacgtgctgcgcgccctggccgacttcggcctgaagcccctgatgcgcgtgtacaccgacagcgacatgagcagcgtgcagtggaagcccctgcgcaagggccaggccgtgcgcacctgggaccgcgacatgttccagcaggccatcgagcgcatgatgagctgggagagctggaaccagcgcgtgggcgaggcctacgccaagctggtggagcagaagagccgcttcgagcagaagaacttcgtgggccaggagcacctggtgcagctggtgaaccagctgcagcaggacatgaaggaggccagccacggcctggagagcaaggagcagaccgcccactacctgaccggccgcgccctgcgcggcagcgacaaggtgttcgagaagtgggagaagctggaccccgacgcccccttcgacctgtacgacaccgagatcaagaacgtgcagcgccgcaacacccgccgcttcggcagccacgacctgttcgccaagctggccgagcccaagtaccaggccctgtggcgcgaggacgccagcttcctgacccgctacgccgtgtacaacagcatcgtgcgcaagctgaaccacgccaagatgttcgccaccttcaccctgcccgacgccaccgcccaccccatctggacccgcttcgacaagctgggcggcaacctgcaccagtacaccttcctgttcaacgagttcggcgagggccgccacgccatccgcttccagaagctgctgaccgtggaggacggcgtggccaaggaggtggacgacgtgaccgtgcccatcagcatgagcgcccagctggacgacctgctgccccgcgacccccacgagctggtggccctgtacttccaggactacggcgccgagcagcacctggccggcgagttcggcggcgccaagatccagtaccgccgcgaccagctgaaccacctgcacgcccgccgcggcgcccgcgacgtgtacctgaacctgagcgtgcgcgtgcagagccagagcgaggcccgcggcgagcgccgccccccctacgccgccgtgttccgcctggtgggcgacaaccaccgcgccttcgtgcacttcgacaagctgagcgactacctggccgagcaccccgacgacggcaagctgggcagcgagggcctgctgagcggcctgcgcgtgatgagcgtggacctgggcctgcgcaccagcgccagcatcagcgtgttccgcgtggcccgcaaggacgagctgaagcccaacagcgagggccgcgtgcccttctgcttccccatcgagggcaacgagaacctggtggccgtgcacgagcgcagccagctgctgaagctgcccggcgagaccgagagcaaggacctgcgcgccatccgcgaggagcgccagcgcaccctgcgccagctgcgcacccagctggcctacctgcgcctgctggtgcgctgcggcagcgaggacgtgggccgccgcgagcgcagctgggccaagctgatcgagcagcccatggacgccaaccagatgacccccgactggcgcgaggccttcgaggacgagctgcagaagctgaagagcctgtacggcatctgcggcgaccgcgagtggaccgaggccgtgtacgagagcgtgcgccgcgtgtggcgccacatgggcaagcaggtgcgcgactggcgcaaggacgtgcgcagcggcgagcgccccaagatccgcggctaccagaaggacgtggtgggcggcaacagcatcgagcagatcgagtacctggagcgccagtacaagttcctgaagagctggagcttcttcggcaaggtgagcggccaggtgtctagaggcggcagcggcagcagcggcggcagcggcggcagcggcggcagcggcgagatgtggcacgagggcctggaggaggccagccgcctgtacttcggcgagcgcaacgtgaagggcatgttcgaggtgctggagcccctgcacgccatgatggagcgcggcccccagaccctgaaggagaccagcttcaaccaggcctacggccgcgacctgatggaggcccaggagtggtgccgcaagtacatgaagagcggcaacgtgaaggacctgacccaggcctgggacctgtactaccacgtgttccgccgcatcagcaagcaggctagccccaagaagaagcgcaaggtg

SEQ ID NO: 22 - DNA, 1461 bp, Artificial Sequence
  Synthetic Construct
cccaagaagaagcgcaaggtgcccgggatgggcgtgcaggtggagaccatcagccccggcgacggccgcaccttccccaagcgcggccagacctgcgtggtgcactacaccggcatgctggaggacggcaagaagttcgacagcagccgcgaccgcaacaagcccttcaagttcatgctgggcaagcaggaggtgatccgcggctgggaggagggcgtggcccagatgagcgtgggccagcgcgccaagctgaccatcagccccgactacgcctacggcgccaccggccaccccggcatcatccccccccacgccaccctggtgttcgacgtggagctgctgaagctggagggcggcagcggcggcagcggcggcggcagcggcggcggccttaagatccgcgccgagaagggcagccgcttcgccatcaccctgcgcgagcacatcgaccacgccaaggaggaccgcctgaagaagctggccgaccgcatcatcatggaggccctgggctacgtgtacgccctggacgacgagcgcggcaagggcaagtgggtggccaagtaccccccctgccagctgatcctgctggaggagctgagcgagtaccagttcaacaacgaccgcccccccagcgagaacaaccagctgatgcagtggagccaccgcggcgtgttccaggagctgctgaaccaggcccaggtgcacgacctgctggtgggcaccatgtacgccgccttcagcagccgcttcgacgcccgcaccggcgcccccggcatccgctgccgccgcgtgcccgcccgctgcgcccgcgagcagaaccccgagcccttcccctggtggctgaacaagttcgtggccgagcacaagctggacggctgccccctgcgcgccgacgacctgatccccaccggcgagggcgagttcttcgtgagccccttcagcgccgaggagggcgacttccaccagatccacgccgacctgaacgccgcccagaacctgcagcgccgcctgtggagcgacttcgacatcagccagatccgcctgcgctgcgactggggcgaggtggacggcgagcccgtgctgatcccccgcaccaccggcaagcgcaccgccgacagctacggcaacaaggtgttctacaccaagaccggcgtgacctactacgagcgcgagcgcggcaagaagcgccgcaaggtgttcgcccaggaggagctgagcgaggaggaggccgagctgctggtggaggccgacgaggcccgcgagaagagcgtggtgctgatgcgcgaccccagcggcatcatcaaccgcggcgactggacccgccagaaggagttctggagcatggtgaaccagcgcatcgagggctacctggtgaagcagatccgcagccgcgtgcgcctgcaggagagcgcctgcgagaacaccggcgacatcgctagccccaagaagaagcgcaaggtg

SEQ ID NO: 23 - DNA, 369 bp, Artificial Sequence
  Synthetic Construct
  misc_feature 342, 343, 344, 345, 346, 347, 348, 349, 350, 351, 352,353, 354, 355, 356, 357, 358, 359, 360, 361: n = A,T,C or G
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggtcgtctataggacggcgagtttttcaacgggtgtgccaatggccactttccaggtggcaaagcccgttgagcttcaaagaagtggcacnnnnnnnnnnnnnnnnnnnntttttttt

SEQ ID NO: 24 - DNA, 89 bp, Artificial Sequence
  Synthetic Construct
  misc_feature 89: A, G, C, or T
  misc_feature 89: Can be present in repeats of any integer between 18 and 35
gtcgtctataggacggcgagtttttcaacgggtgtgccaatggccactttccaggtggcaaagcccgttgagcttcaagaagtggcacn

SEQ ID NO: 25 - DNA, 64 bp, Artificial Sequence
  Synthetic Construct
  misc_feature 64: A, G, C, or T
  misc_feature 64: Can be present in repeats of any integer between 18 and 35
caacgggtgtgccaatggccactttccaggtggcaaagcccgttgagcttcaagaagtggcacn

SEQ ID NO: 26 - DNA, 73 bp, Artificial Sequence
  Synthetic Construct
  misc_feature 73: A, G, C, or T
  misc_feature 73: Can be present in repeats of any integer between 18 and 35
gtcgtctataggacggcgagtttttgtgccaatggccactttccaggtggcaaaagcttcaagaagtggcacn

SEQ ID NO: 27 - DNA, 62 bp, Artificial Sequence
  Synthetic Construct
  misc_feature 62: A, G, C, or T
  misc_feature 62: Can be present in repeats of any integer between 18 and 35
gtcgtctataggacggcgagtttttcaacgggtgtgcccgttgagcttcaagaagtggcacn

SEQ ID NO: 28 - DNA, 6 bp, Artificial Sequence
  Synthetic Construct
  misc_feature 1, 2: n = A,T,C or G
nngrrt

SEQ ID NO: 29 - DNA, 8 bp, Artificial Sequence
  Synthetic Construct
  misc_feature 1, 2, 3, 4: n = A,T,C or G
nnnnryac

SEQ ID NO: 30 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 31 - protein, 108 aa, Artificial Sequence
  Synthetic Construct
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 32 - protein, 90 aa, Artificial Sequence [sequence omitted]

SEQ ID NO: 33 - protein, 1129 aa, Alicyclobacillus acidiphilus
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 34 - protein, 9 aa, Artificial Sequence
  Synthetic Construct
PKKKRKVPG

SEQ ID NO: 35 - protein, 9 aa, Artificial Sequence
  Synthetic Construct
ASPKKKRKV

SEQ ID NO: 36 - RNA, 101 nt, Artificial Sequence
  Synthetic Construct
gucuaaaggacagaauuuuucaacgggugugccaauggccacuuuccagguggcaaagcccguugaacuucucaaaaagaacgcucgcucaguguucugac

SEQ ID NO: 37 - RNA, 37 nt, Artificial Sequence
  Synthetic Construct
  misc_feature 37: A, G, C, or U
  misc_feature 37: Can be present in repeats of any integer between 18 and 35
gucggaucacugagcgagcgaucugagaaguggcacn

SEQ ID NO: 38 - RNA, 138 nt, Artificial Sequence
  Synthetic Construct
  misc_feature 138: A, G, C, or U
  misc_feature 138: Can be present in repeats of any integer between 18 and 35
gucuaaaggacagaauuuuucaacgggugugccaauggccacuuuccagguggcaaagcccguugaacuucucaaaaagaacgcucgcucaguguucugacgucggaucacugagcgagcgaucugagaaguggcacn

SEQ ID NO: 39 - RNA, 142 nt, Artificial Sequence
  Synthetic Construct
  misc_feature 142: A, G, C, or U
  misc_feature 142: Can be present in repeats of any integer between 18 and 35
aacugucuaaaggacagaauuuuucaacgggugugccaauggccacuuuccagguggcaaagcccguugaacuucucaaaaagaacgcucgcucaguguucugacgucggaucacugagcgagcgaucugagaaguggcacn

SEQ ID NO: 40 - RNA, 140 nt, Artificial Sequence
  Synthetic Construct
  misc_feature 140: A, G, C, or U
  misc_feature 140: Can be present in repeats of any integer between 18 and 35
cugucuaaaggacagaauuuuucaacgggugugccaauggccacuuuccagguggcaaagcccguugaacuucucaaaaagaacgcucgcucaguguucugacgucggaucacugagcgagcgaucugagaaguggcacn

SEQ ID NO: 41 - RNA, 128 nt, Artificial Sequence
  Synthetic Construct
  misc_feature 128: A, G, C, or U
  misc_feature 128: Can be present in repeats of any integer between 18 and 35
gucuaaaggacagaauuuuucaacgggugugccaauggccacuuuccagguggcaaagcccguugaacuucucaaaaagaacgcucgcucaguguuaucacugagcgagcgaucugagaaguggcacn

SEQ ID NO: 42 - RNA, 100 nt, Artificial Sequence
  Synthetic Construct
  misc_feature 100: A, G, C, or U
  misc_feature 100: Can be present in repeats of any integer between 18 and 35
gucuaaaggacagaauuuuucaacgggugugccaauggccacuuuccagguggcaaagcccguugaacuucucaaaaagaacgaucugagaaguggcacn

SEQ ID NO: 43 - RNA, 94 nt, Artificial Sequence
  Synthetic Construct
  misc_feature 94: A, G, C, or U
  misc_feature 94: Can be present in repeats of any integer between 18 and 35
gucuaaaggacagaauuuuucaacgggugugccaauggccacuuuccagguggcaaagcccguugaacuucucaaaaagcugagaaguggcacn

SEQ ID NO: 44 - RNA, 92 nt, Artificial Sequence
  Synthetic Construct
  misc_feature 92: A, G, C, or U
  misc_feature 92: Can be present in repeats of any integer between 18 and 35
gucuaaaggacagaauuuuucaacgggugugccaauggccacuuuccagguggcaaagcccguugaacuucucaaagcugagaaguggcacn

SEQ ID NO: 45 - RNA, 92 nt, Artificial Sequence
  Synthetic Construct
  misc_feature 92: A, G, C, or U
  misc_feature 92: Can be present in repeats of any integer between 18 and 35
gucuaaaggacagaauuuuucaacgggugugccaauggccacuuuccagguggcaaagcccguugaacuucucaaaacugagaaguggcacn

SEQ ID NO: 46 - RNA, 90 nt, Artificial Sequence
  Synthetic Construct
  misc_feature 90: A, G, C, or U
  misc_feature 90: Can be present in repeats of any integer between 18 and 35
gucuaaaggacagaauuuuucaacgggugugccaauggccacuuuccagguggcaaagcccguugaacuucucaagcgagaaguggcacn

SEQ ID NO: 47 - RNA, 88 nt, Artificial Sequence
  Synthetic Construct
  misc_feature 88: A, G, C, or U
  misc_feature 88: Can be present in repeats of any integer between 18 and 35
gucuaaaggacagaauuuuucaacgggugugccaauggccacuuuccagguggcaaagcccguugaacuucuaagcagaaguggcacn

SEQ ID NO: 48 - RNA, 86 nt, Artificial Sequence
  Synthetic Construct
  misc_feature 86: A, G, C, or U
  misc_feature 86: Can be present in repeats of any integer between 18 and 35
gucuaaaggacagaauuuuucaacgggugugccaauggccacuuuccagguggcaaagcccguugaacuucaagcgaaguggcacn

SEQ ID NO: 49 - DNA, 20 bp, Artificial Sequence
  Synthetic Construct
tccttctcctgaacaccttc

SEQ ID NO: 50 - DNA, 20 bp, Artificial Sequence
  Synthetic Construct
tttggcctgaataattgcag

SEQ ID NO: 51 - DNA, 20 bp, Artificial Sequence
  Synthetic Construct
cccttcagctaaaataaagg

SEQ ID NO: 52 - DNA, 20 bp, Artificial Sequence
  Synthetic Construct
tagtcatggtgttcttcaac

SEQ ID NO: 53 - DNA, 20 bp, Artificial Sequence
  Synthetic Construct
gctctcaagacccacaatcc

SEQ ID NO: 54 - DNA, 20 bp, Artificial Sequence
  Synthetic Construct
aatcagagaggatcttccga

SEQ ID NO: 55 - DNA, 20 bp, Artificial Sequence
  Synthetic Construct
cggcctcgccctccccagac

SEQ ID NO: 56 - DNA, 20 bp, Artificial Sequence
  Synthetic Construct
gacgctgtctggggagggcg

SEQ ID NO: 57 - DNA, 20 bp, Artificial Sequence
  Synthetic Construct
ttcttcatccctagccagcc

SEQ ID NO: 58 - DNA, 20 bp, Artificial Sequence
  Synthetic Construct
ccttgtcaaggctattggtc

SEQ ID NO: 59 - DNA, 20 bp, Artificial Sequence
  Synthetic Construct
gccagggaccgtttcagaca

SEQ ID NO: 60 - DNA, 20 bp, Artificial Sequence
  Synthetic Construct
agacagatatttgcattgag

SEQ ID NO: 61 - DNA, 23 bp, Artificial Sequence
  Synthetic Construct
gcagctctcattttccatacagt

SEQ ID NO: 62 - DNA, 22 bp, Artificial Sequence
  Synthetic Construct
gatcgggtgtaaactgagcttg

SEQ ID NO: 63 - DNA, 23 bp, Artificial Sequence
  Synthetic Construct
tctgctactcgggaatcctaaaa

SEQ ID NO: 64 - DNA, 23 bp, Artificial Sequence
  Synthetic Construct
tgtcacaagcccacagatatttc

SEQ ID NO: 65 - DNA, 20 bp, Artificial Sequence
  Synthetic Construct
ctcctgctcggtgaatttgg

SEQ ID NO: 66 - DNA, 23 bp, Artificial Sequence
  Synthetic Construct
tagttgataagcgaacctcacac

SEQ ID NO: 67 - DNA, 25 bp, Artificial Sequence
  Synthetic Construct
ggagctgtaggcgattatagttgaa

SEQ ID NO: 68 - DNA, 24 bp, Artificial Sequence
  Synthetic Construct
ttctcaaaccctggaaagcacttt

SEQ ID NO: 69 - DNA, 23 bp, Artificial Sequence
  Synthetic Construct
acttccaccctctgtcttatctc

SEQ ID NO: 70 - DNA, 23 bp, Artificial Sequence
  Synthetic Construct
ccagcttcctcaaatcttatgca

SEQ ID NO: 71 - protein, 18 aa, Artificial Sequence
  Synthetic Construct
SRGGSGSSGGSGGSGGSG

SEQ ID NO: 72 - DNA, 20 bp, Artificial Sequence
  Synthetic Construct
tgcccagctgagtgaactgc

SEQ ID NO: 73 - DNA, 22 bp, Artificial Sequence
  Synthetic Construct
tgcagtcaccatcttctgccag

SEQ ID NO: 74 - DNA, 21 bp, Artificial Sequence
  Synthetic Construct
atcaccgacttcaacagcgtg

SEQ ID NO: 75 - DNA, 20 bp, Artificial Sequence
  Synthetic Construct
tgacagttgagcacacgcag

SEQ ID NO: 76 - DNA, 23 bp, Artificial Sequence
  Synthetic Construct
aactgggacgacatggagaaaat

SEQ ID NO: 77 - DNA, 23 bp, Artificial Sequence
  Synthetic Construct
gaaggtctcaaacatgatctggg

SEQ ID NO: 78 - DNA, 40 bp, Artificial Sequence
  Synthetic Construct
ctacaacattgtccttctcctgaacaccttccaggaattc

SEQ ID NO: 79 - DNA, 36 bp, Artificial Sequence
  Synthetic Construct
ctacaacattgtccttctcctgaacaccaggaattc

SEQ ID NO: 80 - DNA, 32 bp, Artificial Sequence
  Synthetic Construct
ctacaacattgtccttctcctgacaggaattc

SEQ ID NO: 81 - DNA, 40 bp, Artificial Sequence
  Synthetic Construct
tccttttatttcccttcagctaaaataaaggaggaggaag

SEQ ID NO: 82 - DNA, 29 bp, Artificial Sequence
  Synthetic Construct
tccttttatttcccttcagctaaaggaag

SEQ ID NO: 83 - protein, 650 aa, Artificial Sequence
  Synthetic Construct
MAIRSIKLKMKTNSGTDSIYLRKALWRTHQLINEGIAYYMNLLTLYRQEAIGDKTKEAYQAELINIIRNQQRNNGSSEEHGSDQEILALLRQLYELIIPSSIGESGDANQLGNKFLYPLVDPNSQSGKGTSNAGRKPRWKRLKEEGNPDWELEKKKDEERKAKDPTVKIFDNLNKYGLLPLFPLFTNIQKDIEWLPLGKRQSVRKWDKDMFIQAIERLLSWESWNRRVADEYKQLKEKTESYYKEHLTGGEEWIEKIRKFEKERNMELEKNAFAPNDGYFITSRQIRGWDRVYEKWSKLPESASPEELWKVVAEQQNKMSEGFGDPKVFSFLANRENRDIWRGHSERIYHIAAYNGLQKKLSRTKEQATFTLPDAIEHPLWIRYESPGGTNLNLFKLEEKQKKNYYVTLSKIIWPSEEKWIEKENIEIPLAPSIQFNRQIKLKQHVKGKQEISFSDYSSRISLDGVLGGSRIQFNRKYIKNHKELLGEGDIGPVFFNLVVDVAPLQETRNGRLQSPIGKALKVISSDFSKVIDYKPKELMDWMNTGSASNSFGVASLLEGMRVMSIDMGQRTSASVSIFEVVKELPKDQEQKLFYSINDTELFAIHKRSFLLNLPGEVVTKNNKQQRQERRKKRQFVRSQIRMLANVLRL

SEQ ID NO: 84 - protein, 462 aa, Artificial Sequence
  Synthetic Construct
ETKKTPDERKKAIHKLMEIVQSYDSWTASQKEVWEKELNLLTNMAAFNDEIWKESLVELHHRIEPYVGQIVSKWRKGLSEGRKNLAGISMWNIDELEDTRRLLISWSKRSRTPGEANRIETDEPFGSSLLQHIQNVKDDRLKQMANLIIMTALGFKYDKEEKDRYKRWKETYPACQIILFENLNRYLFNLDRSRRENSRLMKWAHRSIPRTVSMQGEMFGLQVGDVRSEYSSRFHAKTGAPGIRCHALTEEDLKAGSNTLKRLIEDGFINESELAYLKKGDIIPSQGGELFVTLSKRYKKDSDNNELTVIHADINAAQNLQKRFWQQNSEVYRVPCQLARMGEDKLYIPKSQTETIKKYFGKGSFVKNNTEQEVYKWEKSEKMKIKTDTTFDLQDLDGFEDISKTIELAQEQQKKYLTMFRDPSGYFFNNETWRPQKEYWSIVNNIIKSCLKKKILSNKVEL

SEQ ID NO: 85 - protein, 1112 aa, Artificial Sequence
  Synthetic Construct
MAIRSIKLKMKTNSGTDSIYLRKALWRTHQLINEGIAYYMNLLTLYRQEAIGDKTKEAYQAELINIIRNQQRNNGSSEEHGSDQEILALLRQLYELIIPSSIGESGDANQLGNKFLYPLVDPNSQSGKGTSNAGRKPRWKRLKEEGNPDWELEKKKDEERKAKDPTVKIFDNLNKYGLLPLFPLFTNIQKDIEWLPLGKRQSVRKWDKDMFIQAIERLLSWESWNRRVADEYKQLKEKTESYYKEHLTGGEEWIEKIRKFEKERNMELEKNAFAPNDGYFITSRQIRGWDRVYEKWSKLPESASPEELWKVVAEQQNKMSEGFGDPKVFSFLANRENRDIWRGHSERIYHIAAYNGLQKKLSRTKEQATFTLPDAIEHPLWIRYESPGGTNLNLFKLEEKQKKNYYVTLSKIIWPSEEKWIEKENIEIPLAPSIQFNRQIKLKQHVKGKQEISFSDYSSRISLDGVLGGSRIQFNRKYIKNHKELLGEGDIGPVFFNLVVDVAPLQETRNGRLQSPIGKALKVISSDFSKVIDYKPKELMDWMNTGSASNSFGVASLLEGMRVMSIDMGQRTSASVSIFEVVKELPKDQEQKLFYSINDTELFAIHKRSFLLNLPGEVVTKNNKQQRQERRKKRQFVRSQIRMLANVLRLETKKTPDERKKAIHKLMEIVQSYDSWTASQKEVWEKELNLLTNMAAFNDEIWKESLVELHHRIEPYVGQIVSKWRKGLSEGRKNLAGISMWNIDELEDTRRLLISWSKRSRTPGEANRIETDEPFGSSLLQHIQNVKDDRLKQMANLIIMTALGFKYDKEEKDRYKRWKETYPACQIILFENLNRYLFNLDRSRRENSRLMKWAHRSIPRTVSMQGEMFGLQVGDVRSEYSSRFHAKTGAPGIRCHALTEEDLKAGSNTLKRLIEDGFINESELAYLKKGDIIPSQGGELFVTLSKRYKKDSDNNELTVIHADINAAQNLQKRFWQQNSEVYRVPCQLARMGEDKLYIPKSQTETIKKYFGKGSFVKNNTEQEVYKWEKSEKMKIKTDTTFDLQDLDGFEDISKTIELAQEQQKKYLTMFRDPSGYFFNNETWRPQKEYWSIVNNIIKSCLKKKILSNKVEL

SEQ ID NO: 86 - protein, 671 aa, Artificial Sequence
  Synthetic Construct
MNIHLKELIRMATKSFILKMKTKNNPQLRLSLWKTHELFNFGVAYYMDLLSLFRQKDLYMHNDEDPDHPVVLKKEEIQERLWMKVRETQQKNGFHGEVSKDEVLETLRALYEELVPSAVGKSGEANQISNKYLYPLTDPASQSGKGTANSGRKPRWKKLKEAGDPSWKDAYEKWEKERQEDPKLKILAALQSFGLIPLFRPFTENDHKAVISVKWMPKSKNQSVRKFDKDMFNQAIERFLSWESWNEKVAEDYEKTVSIYESLQKELKGISTKAFEIMERVEKAYEAHLREITFSNSTYRIGNRAIRGWTEIVKKWMKLDPSAPQGNYLDVVKDYQRRHPRESGDFKLFELLSRPENQAAWREYPEFLPLYVKYRHAEQRMKTAKKQATFTLCDPIRHPLWVRYEERSGTNLNKYRLIMNEKEKVVQFDRLICLNADGHYEEQEDVTVPLAPSQQFDDQIKFSSEDTGKGKHNFSYYHKGINYELKGTLGGARIQFDREHLLRRQGVKAGNVGRIFLNVTLNIEPMQPFSRSGNLQTSVGKALKVYVDGYPKVVNFKPKELTEHIKESEKNTLTLGVESLPTGLRVMSVDLGQRQAAAISIFEVVSEKPDDNKLFYPVKDTDLFAVHRTSFNIKLPGEKRTERRMLEQQKRDQAIRDLSRKLKFLKNVLNM

SEQ ID NO: 87 - protein, 471 aa, Artificial Sequence
  Synthetic Construct
QKLEKTDEREKRVNRWIKDREREEENPVYVQEFEMISKVLYSPHSVWVDQLKSIHRKLEEQLGKEISKWRQSISQGRQGVYGISLKNIEDIEKTRRLLFRWSMRPENPGEVKQLQPGERFAIDQQNHLNHLKDDRIKKLANQIVMTALGYRYDGKRKKWIAKHPACQLVLFEDLSRYAFYDERSRLENRNLMRWSRREIPKQVAQIGGLYGLLVGEVGAQYSSRFHAKSGAPGIRCRVVKEHELYITEGGQKVRNQKFLDSLVENNIIEPDDARRLEPGDLIRDQGGDKFATLDERGELVITHADINAAQNLQKRFWTRTHGLYRIRCESREIKDAVVLVPSDKDQKEKMENLFGIGYLQPFKQENDVYKWVKGEKIKGKKTSSQSDDKELVSEILQEASVMADELKGNRKTLFRDPSGYVFPKDRWYTGGRYFGTLEHLLKRKLAERRLFDGGSSRRGLFNGTDSNTNVE

SEQ ID NO: 88 - protein, 1142 aa, Artificial Sequence
  Synthetic Construct
MNIHLKELIRMATKSFILKMKTKNNPQLRLSLWKTHELFNFGVAYYMDLLSLFRQKDLYMHNDEDPDHPVVLKKEEIQERLWMKVRETQQKNGFHGEVSKDEVLETLRALYEELVPSAVGKSGEANQISNKYLYPLTDPASQSGKGTANSGRKPRWKKLKEAGDPSWKDAYEKWEKERQEDPKLKILAALQSFGLIPLFRPFTENDHKAVISVKWMPKSKNQSVRKFDKDMFNQAIERFLSWESWNEKVAEDYEKTVSIYESLQKELKGISTKAFEIMERVEKAYEAHLREITFSNSTYRIGNRAIRGWTEIVKKWMKLDPSAPQGNYLDVVKDYQRRHPRESGDFKLFELLSRPENQAAWREYPEFLPLYVKYRHAEQRMKTAKKQATFTLCDPIRHPLWVRYEERSGTNLNKYRLIMNEKEKVVQFDRLICLNADGHYEEQEDVTVPLAPSQQFDDQIKFSSEDTGKGKHNFSYYHKGINYELKGTLGGARIQFDREHLLRRQGVKAGNVGRIFLNVTLNIEPMQPFSRSGNLQTSVGKALKVYVDGYPKVVNFKPKELTEHIKESEKNTLTLGVESLPTGLRVMSVDLGQRQAAAISIFEVVSEKPDDNKLFYPVKDTDLFAVHRTSFNIKLPGEKRTERRMLEQQKRDQAIRDLSRKLKFLKNVLNMQKLEKTDEREKRVNRWIKDREREEENPVYVQEFEMISKVLYSPHSVWVDQLKSIHRKLEEQLGKEISKWRQSISQGRQGVYGISLKNIEDIEKTRRLLFRWSMRPENPGEVKQLQPGERFAIDQQNHLNHLKDDRIKKLANQIVMTALGYRYDGKRKKWIAKHPACQLVLFEDLSRYAFYDERSRLENRNLMRWSRREIPKQVAQIGGLYGLLVGEVGAQYSSRFHAKSGAPGIRCRVVKEHELYITEGGQKVRNQKFLDSLVENNIIEPDDARRLEPGDLIRDQGGDKFATLDERGELVITHADINAAQNLQKRFWTRTHGLYRIRCESREIKDAVVLVPSDKDQKEKMENLFGIGYLQPFKQENDVYKWVKGEKIKGKKTSSQSDDKELVSEILQEASVMADELKGNRKTLFRDPSGYVFPKDRWYTGGRYFGTLEHLLKRKLAERRLFDGGSSRRGLFNGTDSNTNVE

SEQ ID NO: 89 - DNA, 20 bp, Artificial Sequence
  Synthetic Construct
tgcttggctgccagtacctg

SEQ ID NO: 90 - DNA, 20 bp, Artificial Sequence
  Synthetic Construct
atcgagacctcaagctgggc

SEQ ID NO: 91 - DNA, 20 bp, Artificial Sequence
  Synthetic Construct
cctgaatgaagatctggagg

SEQ ID NO: 92 - DNA, 20 bp, Artificial Sequence
  Synthetic Construct
ctcctcttgtgcagctccag

SEQ ID NO: 93 - DNA, 20 bp, Artificial Sequence
  Synthetic Construct
gccgtaggtagtatcgggcc

SEQ ID NO: 94 - DNA, 20 bp, Artificial Sequence
  Synthetic Construct
cggtgcaggtactggcagcc

SEQ ID NO: 95 - DNA, 20 bp, Artificial Sequence
  Synthetic Construct
tcacctccagatcttcattc

SEQ ID NO: 96 - RNA, 90 nt, Artificial Sequence
  Synthetic Construct
  misc_feature 90: n = A,U,C or G
  misc_feature 90: Can be present in repeats of any integer between 18 and 35
gucgucuauaggacggcgaguuuuucaacgggugugccaauggccacuuuccagguggcaaagcccguugagcuucaaagaaguggcacn

SEQ ID NO: 97 - RNA, 65 nt, Artificial Sequence
  Synthetic Construct
  misc_feature 65: n = A,U,C or G
  misc_feature 65: Can be present in repeats of any integer between 18 and 35
caacgggugugccaauggccacuuuccagguggcaaagcccguugagcuucaaagaaguggcacn

SEQ ID NO: 98 - RNA, 74 nt, Artificial Sequence
  Synthetic Construct
  misc_feature 74: n = A,U,C or G
  misc_feature 74: Can be present in repeats of any integer between 18 and 35
gucgucuauaggacggcgaguuuuugugccaauggccacuuuccagguggcaaaagcuucaaagaaguggcacn

SEQ ID NO: 99 - RNA, 63 nt, Artificial Sequence
  Synthetic Construct
  misc_feature 63: n = A,U,C or G
  misc_feature 63: Can be present in repeats of any integer between 18 and 35
gucgucuauaggacggcgaguuuuucaacgggugugcccguugagcuucaaagaaguggcacn

SEQ ID NO: 100 - RNA, 138 nt, Artificial Sequence; identical to SEQ ID NO: 38
  Synthetic Construct
  misc_feature 138: n = A,U,C or G
  misc_feature 138: Can be present in repeats of any integer between 18 and 35

SEQ ID NO: 101 - RNA, 86 nt, Artificial Sequence; identical to SEQ ID NO: 48
  Synthetic Construct
  misc_feature 86: n = A,U,C or G
  misc_feature 86: Can be present in repeats of any integer between 18 and 35

(19) United States
(12) Patent Application Publication
(10) Pub. No.: US 2023/0323322 A1
(43) Pub. Date: Oct. 12, 2023

(54) SPLIT CAS12 SYSTEMS AND METHODS OF USE THEREOF

(71) Applicants: Institute of Zoology, Chinese Academy of Sciences, Beijing (CN); Beijing Institute for Stem Cell and Regenerative Medicine, Beijing (CN)

(72) Inventors: Qi ZHOU, Beijing (CN); Wei LI, Beijing (CN); Fei TENG, Beijing (CN); Qingqin GAO, Beijing (CN)

(21) Appl. No.: 18/023,171

(22) PCT Filed: Aug. 24, 2021
(86) PCT No.: PCT/CN2021/114339

§ 371 Date: Feb. 24, 2023

(30) Foreign Application Priority Data
Aug. 25, 2020 (WO) PCT/CN2020/111057

(51) Int. Cl. C12N 9/22; C12N 15/10

(52) U.S. Cl. CPC C12N 9/22; C12N 15/102; C12N 2310/20

(57) ABSTRACT

Provided are engineered split Cas12b systems and methods of use thereof. Also provided are compositions comprising one or more components of the engineered split Cas12b systems, as well as engineered cells and non-human animals produced by the methods. The systems, methods and compositions are useful for genome editing, transcription modulation and gene therapy.

### CROSS REFERENCE TO RELATED APPLICATIONS

This application claims priority benefit of International Patent Application No. PCT/CN2020/111057 filed Aug. 25, 2020, the contents of which are incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 182452000441SEQLIST.txt, date recorded: Aug. 23, 2021, size: 156 KB).

### FIELD

The present application relates generally to the field of biotechnology. More specifically, the present application relates to an engineered CRISPR-Cas system.

### BACKGROUND

Genome editing is an important and useful technology in genomic research and various applications. Various systems may be used for genome editing, including the clustered regularly interspersed short palindromic repeats (CRISPR)-Cas system, the transcription activator-like effector nuclease (TALEN) system, and the zinc finger nuclease (ZFN) system.

The CRISPR-Cas system is an efficient and cost-effective genome-editing technology that is widely applicable in a range of eukaryotic organisms from yeast and plants to zebrafish and human (reviewed by Van der Oost 2013, Science 339: 768-770, and Charpentier and Doudna, 2013, Nature 495: 50-51). The CRISPR-Cas system provides adaptive immunity in archaea and bacteria by employing a combination of Cas effector proteins and CRISPR RNAs (crRNAs). To date, two classes (class 1 and 2) including six types (type I-VI) of CRISPR-Cas systems have been characterized according to prominent functional and evolutionary modularity of the systems. Among class 2 CRISPR-Cas systems, type II Cas9 systems and type V-A Cas12a/Cpf1 systems have been harnessed for genome editing, and hold tremendous promise for biomedical research.

However, current CRISPR-Cas systems have various limitations. For instance, they may be limited in their efficiency, ease of use, stability, specificity, etc. Accordingly, there exists a need for improved methods and systems for effective genome editing.

### BRIEF SUMMARY

To address the above and other needs, the present disclosure provides engineered CRISPR-Cas systems comprising split Cas12b polypeptides and methods of use thereof.

In one aspect, the present application provides an engineered Clustered Regularly Interspersed Short Palindromic Repeat (CRISPR)-CRISPR associated (Cas) (CRISPR-Cas) system comprising: (a) a first polypeptide comprising a N-terminal portion of a reference Cas12b protein, (b) a second polypeptide comprising a C-terminal portion of the reference Cas12b protein, and (c) a guide RNA comprising a guide sequence; wherein the reference Cas12b protein comprises from the N-terminus to the C-terminus: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III) and a second Nuc domain (Nuc-II), wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; and wherein the first polypeptide, the second polypeptide and the guide RNA are capable of associating with each other to form a CRIPSR complex that specifically binds to a target nucleic acid comprising a target sequence complementary to the guide sequence. In some embodiments, wherein the reference Cas12b protein has N amino acid residues; the first polypeptide comprises amino acid residues 1 to X of the reference Cas12b protein, wherein X is an integer greater than 1 and smaller than N; and the second polypeptide comprises amino acid residues X+1 to N of the reference Cas12b protein.

In some embodiments according to any one of the CRISPR-Cas systems described above, the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I and BH domains of the reference Cas12b protein, wherein the C-terminal portion of the reference Cas12b protein comprises RuvC-II, Nuc-I, RuvC-III and Nuc-II domains of the reference Cas12b protein, and wherein REC2 domain of the reference Cas12b protein is split between the N-terminal portion of the reference Cas12b protein and the C-terminal portion of the reference Cas12b protein. In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein.

In some embodiments according to any one of the CRISPR-Cas systems described above, the reference Cas12b protein is a Cas12b protein selected from the group consisting of Cas12b from Alicyclobacillus acidiphilus (AaCas12b), Cas12b from Alicyclobacillus kakegawensis (AkCas12b), Cas12b from Alicyclobacillus macrosporangiidus (AmCas12b), Cas12b from Bacillus hisashii (BhCas12b), BsCas12b from Bacillus, Cas12b from Bacillus sp. V3-13 (Bs3Cas12b), Cas12b from Desulfovibrio inopinatus (DiCas12b), Cas12b from Laceyella sediminis (LsCas12b), Cas 12b from Spirochaetes bacterium (SbCas12b), Cas12b from Tuberibacillus calidus (TcCas12b) and functional derivatives thereof.

In some embodiments according to any one of the CRISPR-Cas systems described above, the reference Cas12b protein is a Cas12b protein from Alicyclobacillus acidiphilus (AaCas12b) or a functional derivative thereof. In some embodiments, the N-terminal portion of the reference Cas12b protein comprises amino acid residues 1 to 658 of the reference Cas12b protein, and the C-terminal portion of the reference Cas12b protein comprises amino acid residues 659 to 1129 of the reference Cas12b protein, wherein the amino acid residue numbering is according to SEQ ID NO: 33. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 3, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 4. In some embodiments, the N-terminal portion of the reference Cas12b protein comprises the amino acid sequence of SEQ ID NO: 3, and wherein the C-terminal portion of the reference Cas12b protein comprises the amino acid sequence of SEQ ID NO: 4. In some embodiments, the N-terminal portion of the reference Cas12b protein comprises amino acid residues 1 to 783 of the reference Cas12b protein, and the C-terminal portion of the reference Cas12b protein comprises amino acid residues 784 to 1129 of the reference Cas12b protein, wherein the amino acid residue numbering is according to SEQ ID NO: 33. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 5, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 6. In some embodiments, the N-terminal portion of the reference Cas12b protein comprises the amino acid sequence of SEQ ID NO: 5, and wherein the C-terminal portion of the reference Cas12b protein comprises the amino acid sequence of SEQ ID NO: 6. In some embodiments, the N-terminal portion of the reference Cas12b protein comprises amino acid residues 1 to 518 of the reference Cas12b protein, and the C-terminal portion of the reference Cas12b protein comprises amino acid residues 519 to 1129 of the reference Cas12b protein, wherein the amino acid residue numbering is according to SEQ ID NO: 33. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 1, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 2. In some embodiments, the N-terminal portion of the reference Cas12b protein comprises the amino acid sequence of SEQ ID NO: 1, and wherein the C-terminal portion of the reference Cas12b protein comprises the amino acid sequence of SEQ ID NO: 2.

In some embodiments according to any one of the CRISPR-Cas systems described above, the reference Cas12b protein is a Cas12b protein from Bacillus sp. V3-13 (Bs3Cas12b) or a functional derivative thereof. In some embodiments, the N-terminal portion of the reference Cas12b protein comprises amino acid residues 1 to 650 of the reference Cas12b protein, and the C-terminal portion of the reference Cas12b protein comprises amino acid residues 651 to 1112 of the reference Cas12b protein, wherein the amino acid residue numbering is according to SEQ ID NO: 85. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 83, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 84. In some embodiments, the N-terminal portion of the reference Cas12b protein comprises the amino acid sequence of SEQ ID NO: 83, and wherein the C-terminal portion of the reference Cas12b protein comprises the amino acid sequence of SEQ ID NO: 84.

In some embodiments according to any one of the CRISPR-Cas systems described above, the reference Cas12b protein is a Cas12b protein from Tuberibacillus calidus (TcCas12b) or a functional derivative thereof. In some embodiments, the N-terminal portion of the reference Cas12b protein comprises amino acid residues 1 to 671 of the reference Cas12b protein, and the C-terminal portion of the reference Cas12b protein comprises amino acid residues 672 to 1142 of the reference Cas12b protein, wherein the amino acid residue numbering is according to SEQ ID NO: 88. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 86, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 87. In some embodiments, the N-terminal portion of the reference Cas12b protein comprises the amino acid sequence of SEQ ID NO: 86, and wherein the C-terminal portion of the reference Cas12b protein comprises the amino acid sequence of SEQ ID NO: 87.

In some embodiments according to any one of the CRISPR-Cas systems described above, the first polypeptide comprises a first dimerization domain, and the second polypeptide comprises a second dimerization domain. In some embodiments, the first dimerization domain and the second dimerization domain associate with each other in the presence of an inducer. In some embodiments, the first dimerization domain is FK506 binding protein (FKBP) and the second dimerization domain is FKBP-rapamycin-binding domain (FRB), or the first dimerization domain is FRB and the second dimerization domain is FKBP, and the inducer is rapamycin. In some embodiments, the first polypeptide and the second polypeptide do not comprise dimerization domains.

In some embodiments according to any one of the CRISPR-Cas systems described above, the guide RNA is a single-guide RNA (sgRNA) comprising a trans-activating CRISPR RNA (tracrRNA) sequence and a CRISPR RNA (crRNA) sequence comprising the guide sequence, and wherein the sgRNA comprises from the 5′ to the 3′: a first stem loop, a second stem loop, a third stem loop and a fourth stem loop. In some embodiments, the sgRNA comprises the nucleic acid sequence of SEQ ID NO: 7. In some embodiments, the sgRNA comprises the nucleic acid sequence of SEQ ID NO: 96. In some embodiments, the sgRNA comprises the nucleic acid sequence of SEQ ID NO: 100.

In some embodiments according to any one of the CRISPR-Cas systems described above, the guide RNA is a truncated sgRNA comprising a tracrRNA sequence and a crRNA sequence comprising the guide sequence, and wherein compared to a full-length sgRNA comprising a wildtype tracrRNA sequence and a wildtype crRNA sequence corresponding to the reference Cas12b protein, the truncated sgRNAs lacks one or more stem loops. In some embodiments, the full-length sgRNA comprises from the 5′ to the 3′: a first stem loop, a second stem loop, a third stem loop and a fourth stem loop, and wherein the truncated sgRNA does not comprise the first stem loop, the second stem loop, and/or the third stem loop. In some embodiments, the truncated sgRNA comprises the nucleic acid sequence selected from the group consisting of SEQ ID NOs: 8-10. In some embodiments, the truncated sgRNA comprises the nucleic acid sequence selected from the group consisting of SEQ ID NOs: 97-99. In some embodiments, the truncated sgRNA comprises the nucleic acid sequence of SEQ ID NO: 101.

In some embodiments according to any one of the CRISPR-Cas systems described above, the reference Cas12b protein is enzymatically active. In some embodiments, the reference Cas12b protein is enzymatically inactive. In some embodiments, the reference Cas12b protein comprises one or more mutations selected from the group consisting of D570A, R785A, R911A, and D977A, wherein the amino acid numbering is according to SEQ ID NO: 33. In some embodiments, the first polypeptide further comprises a functional domain fused to the N-terminal portion of the reference Cas12b protein, and/or the second polypeptide further comprises a functional domain fused to the C-terminal portion of the reference Cas12b protein. In some embodiments, the functional domain is selected from the group consisting of a translation initiator domain, a transcription repressor domain, a transactivation domain, an epigenetic modification domain, and a nuclease domain. In some embodiments, the functional domain is a transcription repressor domain, such as one or more functional domains selected from the group consisting of Krüppel associated box (KRAB), EnR, NuE, NcoR, SID, and SID4X. In some embodiments, the functional domain is a transactivation domain, such as one or more functional domains selected from the group consisting of VP64, p65, HSF1, VP16, MyoD1, HSF1, RTA, SETT/9, and combinations thereof. In some embodiments, the first polypeptide comprises from the N-terminus to the C-terminus: a first functional domain, the N-terminal portion of the reference Cas12b protein, a second functional domain; and/or wherein the second polypeptide comprises from the N-terminus to the C-terminus: a third functional domain, the C-terminal portion of the reference Cas12b protein, a fourth functional domain.

In some embodiments according to any one of the CRISPR-Cas systems described above, the first polypeptide and/or the second polypeptide further comprises a nuclear localization signal (NLS).

In some embodiments according to any one of the CRISPR-Cas systems described above, the engineered CRISPR-Cas system comprises a first nucleic acid encoding the first polypeptide and a second nucleic acid encoding the second polypeptide. In some embodiments, the first nucleic acid is present in a first vector, and the second nucleic acid is present in a second vector. In some embodiments, the first vector and the second vector are adeno-associated viral (AAV) vectors. In some embodiments, the first vector or the second vector further comprises a third nucleic acid encoding the guide RNA. In some embodiments, the engineered CRISPR-Cas system comprises a third vector comprising a third nucleic acid encoding the guide RNA.

Another aspect of the present application provides a method of modifying a target nucleic acid in a cell, comprising: contacting the cell with any one of the engineered CRISPR-Cas systems described above, wherein the guide sequence of the guide RNA is complementary to a target sequence of the target nucleic acid, wherein the first polypeptide, the second first polypeptide and the guide RNA associate with each other to bind to the target nucleic acid, thereby modifying the target nucleic acid. In some embodiments, the method does not comprise contacting the cell with an inducer. In some other embodiments, the method further comprises contacting the cell with an inducer.

In some embodiments according to any one of the methods described above, the cell is a bacterial cell, a yeast cell, a plant cell, or an animal cell (e.g., a mammalian cell).

In some embodiments according to any one of the methods described above, the target nucleic acid is cleaved or the target sequence in the target nucleic acid is altered by the engineered CRISPR-Cas system. In some embodiments, the method further comprises contacting the target nucleic acid with a donor DNA. In some embodiments, expression of the target nucleic acid is altered by the engineered CRISPR-Cas system.

In some embodiments according to any one of the methods described above, the method is carried out ex vivo. In some embodiments, the method is carried out in vivo.

In some embodiments according to any one of the methods described above, the target nucleic acid is a genomic DNA. In some embodiments, the target sequence is associated with a disease or condition.

In some embodiments according to any one of the methods described above, the guide RNA comprises a plurality of crRNA sequences, wherein each crRNA comprises a different target sequence.

In yet another aspect, the present application provides a method of treating a disease or condition associated with a target nucleic acid in cells of an individual, comprising modifying the target nucleic acid in the cells of the individual using any one of the methods described above. In some embodiments, the disease or condition is selected from the group consisting of cancer, cardiovascular diseases, hereditary diseases, autoimmune diseases, metabolic diseases, neurodegenerative diseases, ocular diseases, bacterial infections and viral infections. In some embodiments, the target nucleic acid is PCSK9, and the disease or condition is a cardiovascular disease.

In still another aspect, the present application provides an engineered cell having a modified target nucleic acid that has been modified using any one of the methods of modifying a target nucleic acid described above. Also provided is an engineered non-human animal comprising one or more engineered cells according to any one of the engineered cells described above.

The present application further provides an engineered polypeptide comprising an amino acid sequence selected from the group consisting of SEQ ID NOs: 1-6, 11-16, 78-79 and 81-82, and an engineered sgRNA comprising a nucleic acid sequence selected from the group consisting of SEQ ID NOs: 7-10.

Also provided are kits, articles of manufacture comprising any one of the engineered polypeptides, engineered sgRNAs, and/or engineered cells described herein.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

### BRIEF DESCRIPTION OF DRAWINGS

FIG. 1 shows a schematic of an exemplary rapamycin-inducible split Cas12b system. Cas12b is split into an N-terminus fragment and a C-terminus fragment, which are fused to FRB and FKBP dimerization domains, respectively. Rapamycin induces dimerization between FKBP and FRB, which enables split Cas12b fragments to re-associate and regain RNA-guided DNA nuclease activity.

FIG. 2 shows constructs of an exemplary rapamycin-inducible split Cas12b system. The top construct encodes a Cas12b N-terminus fragment fused to an FRB domain. The bottom construct encodes an FKBP domain fused to a Cas12b C-terminus fragment.

FIG. 3 shows three exemplary pairs of split Cas12b polypeptides based on the Alicyclobacillus acidiphilus Cas12b (AaCas12b). Split 1 Cas12b polypeptides split a full-length AaCas12b protein at amino acid position 518. Split 2 Cas12b polypeptides split a full-length AaCas12b protein at amino acid position 658. Split 3 Cas12b polypeptides splits a full-length AaCas12b at amino acid position 783.

FIG. 4A shows results of a T7 endonuclease I (T7EI) assay assessing the Insertion-Deletion (Indel) mutations at human target sites induced by three split AaCas12b protein constructs of FIG. 3. The human target sites include CCR5-1, CCR5-2, DNMT1, RNF2, and VEGFA. The split AaCas12b proteins comprise dimerization domains.

FIG. 4B shows DNA sequences at the CCR5-10 locus and DNMT1-16 locus, and exemplary edited sequences using the split AsCas12b systems of FIG. 4A. Deleted bases are shown as dashes; PAM sequence is boxed and spacer sequence is underlined.

FIG. 5 shows an exemplary rapamycin-inducible split Cas12b-based gene activation system, wherein the reference Cas12b is a catalytically dead Cas12b (dCas12b).

FIG. 6 shows an exemplary rapamycin-inducible split Cas12b-based gene repression system, wherein the reference Cas12b is a catalytically dead Cas12b (dCas12b).

FIG. 7 shows an exemplary auto-inducing split self-inducing split Cas12b-based gene activation system, wherein the reference Cas12b is a catalytically dead Cas12b (dCas12b).

FIG. 8 shows an exemplary auto-inducing split Cas12b-based gene repression system, wherein the reference Cas12b is a catalytically dead Cas12b (dCas12b).

FIG. 9 shows a DNA sequence corresponding to an exemplary sgRNA scaffold, artsgRNA13. Secondary structures of the sgRNA are annotated.

FIG. 10 shows DNA sequences corresponding to exemplary truncated sgRNA scaffolds, artsgRNA13Δloop1, artsgRNA13Δloop2, and artsgRNA13Δloop3. Secondary structures of the sgRNAs are annotated.

FIG. 11 shows transcriptional activation of the HBG gene using an exemplary split Cas12b-based gene activation system.

FIG. 12 shows transcriptional repression of the PCSK9 gene using an exemplary split Cas12b-based gene repression system.

FIG. 13 shows results of a T7EI assay assessing the Indel mutations at PLK-1 human target sites induced by three split AaCas12b protein constructs of FIG. 3. The split AaCas12b proteins contain no dimerization domains. AasgRNA3.8 has the nucleic acid sequence of SEQ ID NO: 101. “WT” indicates blank control.

FIG. 14 shows results of a T7EI assay assessing the Indel mutations at PLK-1 human target sites induced by Split 2 AaCas12b protein constructs and corresponding split protein constructs of orthologues Bs3Cas12b and TcCas12b. The Split2 BsCas12b and TcCas12b proteins contain no dimerization domains. “WT” indicates blank control.

### DETAILED DESCRIPTION

The present application provides engineered CRISPR-Cas systems (also referred herein as “split Cas12b systems”) comprising split Cas12b polypeptides, which can reconstitute into a functional Cas12b protein upon binding to an inducer or upon binding to a guide RNA (i.e., auto-induction). Advantages of such engineered split Cas12b systems include, but are not limited to: (1) reduced construct sizes, which facilitate delivery of the split Cas12b system into cells via AAV vectors; (2) flexibility of including additional functional domains in the split Cas12b polypeptides, which can be used for transcriptional regulation and other sequence-specific gene modifications; and (3) multiplex genome editing at a plurality of genomic target sites. In some embodiments, truncated gRNAs are used in combination with an inducer-controlled split Cas12b system, which can minimize auto-induction of the split Cas12b system, allow tighter control over the split Cas12b system, and reduce off-target editing events.

Accordingly, one aspect of the present application provides an engineered CRISPR-Cas system comprising: (a) a first polypeptide comprising a N-terminal portion of a reference Cas12b protein, (b) a second polypeptide comprising a C-terminal portion of the reference Cas12b protein, and (c) a guide RNA comprising a guide sequence; wherein the reference Cas12b protein comprises from the N-terminus to the C-terminus: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III) and a second Nuc domain (Nuc-II), wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; and wherein the first polypeptide, the second polypeptide and the guide RNA are capable of associating with each other to form a CRIPSR complex that specifically binds to a target nucleic acid comprising a target sequence complementary to the guide sequence. In some embodiments, the first polypeptide comprises a first dimerization domain, and the second polypeptide comprises a second dimerization domain. In some embodiments, the first dimerization domain and the second dimerization domain associate with each other in the presence of an inducer. In some embodiments, the guide RNA is a truncated guide RNA as compared to the wild-type guide RNA, and the truncated guide RNA is capable of associating with the first polypeptide and the second polypeptide to form a functional CRISPR complex. In some embodiments, the first dimerization domain and the second dimerization domain are capable of associating with each other in the absence of any inducer.

Also provided are compositions, kits and articles of manufacture comprising one or more components of the engineered CRISPR-Cas systems, as well as engineered cells and animals produced by the methods of using the systems.

### Definitions

Unless defined otherwise, all technical and scientific terms used herein have the same meaning as is commonly understood by one of ordinary skill in the art to which this disclosure belongs.

As used herein, a “split Cas12b polypeptide” refers to one or a pair of polypeptides, each of which comprises a portion of a functional Cas12b protein (e.g., a full-length Cas12b protein or a functional variant thereof; also referred herein as a “reference Cas12b protein”), and the pair of polypeptides can associate with each other to reconstitute the functional Cas12b protein. A functional Cas12b protein has one or more activities selected from DNA binding, cleavage of a single strand of a duplex nucleic acid (also referred herein as “nickase activity)” and cleavage of both strands of a duplex nucleic acid. The portion of the functional Cas12b protein within each split Cas12b polypeptide is referred herein as the “split Cas12b portion.” For split Cas12b polypeptide systems having two split Cas12b portions, there are two split Cas12b portions, i.e., an N-terminal split Cas12b portion and a C-terminal split Cas12b portion. The N-terminal split Cas12b portion may comprise amino acid residues from the first amino acid residue at the N-terminus of a reference Cas12b polypeptide to the amino acid residue at the split position at amino acid residue X in the reference Cas12b polypeptide; and the C-terminal split Cas12b portion may comprise amino acid residue X+1 in the reference Cas12b polypeptide to the last amino acid residue in the reference Cas12b polypeptide. It is also contemplated that amino acid residue addition(s), deletion(s) and insertion(s) with respect to the reference Cas12b polypeptide, including at the N-terminus, the C-terminus, at the boundary of the split position, and/or at internal position(s), may be applicable to the N-terminal split Cas12b portion and/or the C-terminal split Cas12b portion, as long as the N-terminal split Cas12b portion and the C-terminal Cas12b portion may reconstitute into a functional Cas12b protein. A “split Cas12b construct” refers to a nucleic acid sequence encoding the split Cas12b polypeptide. In some embodiments, one or both split Cas12b polypeptides of the pair comprises additional functional domains fused to the split Cas12b portion. In some embodiments, the split Cas12b polypeptides do not have additional function domains fused to the split Cas12b portions. In embodiments of inducer-controlled split Cas12b systems, the split Cas12b polypeptides may associate with each other in the presence of an inducer. In embodiments of auto-inducing or self-inducing split Cas12b systems, the split Cas12b polypeptides may associate with each other without any inducer. Certain inducer-controlled split Cas12b systems may also have auto-inducing activity.

As used herein, “guide RNA” and “gRNA” are used herein interchangeably to refer to RNA that is capable of forming a complex with a Cas protein (e.g., a reconstituted Cas12b protein from two split Cas12b polypeptides or portions) and a target nucleic acid (e.g., duplex DNA). A guide RNA may comprise a single RNA molecule or two or more RNA molecules associated with each other via hybridization of complementary regions in the two or more RNA molecules. When used in connection with a Cas12b protein, a guide RNA comprises a crRNA and a tracrRNA. The “crRNA” or “CRISPR RNA” comprises a guide sequence that has sufficient complementarity to a target sequence of a target nucleic acid (e.g., duplex DNA), which guides sequence-specific binding of the CRISPR complex (i.e., Cas12b+crRNA+tracrRNA complex) to the target nucleic acid. The “tracrRNA” or “trans-activating CRISPR RNA” is partially complementary to and base pairs with the crRNA, and may play a role in the maturation of the crRNA. A “single guide RNA” or “sgRNA” is an engineered guide RNA having both crRNA and tracrRNA fused to each other in a single molecule.

The terms “nucleic acid,” “polynucleotide,” and “nucleotide sequence” are used interchangeably to refer to a polymeric form of nucleotides of any length, including deoxyribonucleotides, ribonucleotides, combinations thereof, and analogs thereof. “Oligonucleotide” and “oligo” are used interchangeably to refer to a short polynucleotide, having no more than about 50 nucleotides.

As used herein, “complementarity” refers to the ability of a nucleic acid to form hydrogen bond(s) with another nucleic acid by traditional Watson-Crick base-pairing. A percent complementarity indicates the percentage of residues in a nucleic acid molecule which can form hydrogen bonds (i.e., Watson-Crick base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence form hydrogen bonds with the same number of contiguous residues in a second nucleic acid sequence. “Substantially complementary” as used herein refers to a degree of complementarity that is at least about any one of 70%, 75%, 80%, 85%, 90%, 95%, 97%, 98%, 99%, or 100% over a region of about 40, 50, 60, 70, 80, 100, 150, 200, 250 or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay,” Elsevier, N,Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. A sequence capable of hybridizing with a given sequence is referred to as the “complement” of the given sequence.

“Percentage (%) sequence identity” with respect to a nucleic acid sequence is defined as the percentage of nucleotides in a candidate sequence that are identical with the nucleotides in the specific nucleic acid sequence, after aligning the sequences by allowing gaps, if necessary, to achieve the maximum percent sequence identity. “Percentage (%) sequence identity” with respect to a peptide, polypeptide or protein sequence is the percentage of amino acid residues in a candidate sequence that are identical substitutions to amino acid residues in the specific peptide or amino acid sequence, after aligning the sequences by allowing gaps, if necessary, to achieve the maximum percent sequence homology. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The terms “polypeptide”, and “peptide” are used interchangeably herein to refer to polymers of amino acids of any length. The polymer may he linear or branched, it may comprise modified amino acids, and it may be interrupted by non-amino acids. A protein may have one or more polypeptides. The terms also encompass an amino acid polymer that has been modified; for example, disulfide bond formation, glycosylation, lipidation, acetylation, phosphorylation, or any other manipulation, such as conjugation with a labeling component.

As used herein, a “variant” is interpreted to mean a polynucleotide or polypeptide that differs from a reference polynucleotide or polypeptide, respectively, but retains essential properties. A typical variant of a polynucleotide differs in nucleic acid sequence from another, reference polynucleotide. Changes in the nucleic acid sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

A “cell” as used herein, is understood to refer not only to the particular individual cell, but to the progeny or potential progeny of the cell. Because certain modifications may occur in succeeding generations due to either mutation or environmental influences, such progeny may not, in fact, be identical to the parent cell, but are still included within the scope of the term as used herein.

The term “transduction” and “transfection” as used herein include all methods known in the art using an infectious agent (such as a virus) or other means to introduce DNA into cells for expression of a protein or molecule of interest. Besides a virus or virus like agent, there are chemical-based transfection methods, such as those using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, delivery of plasmids, or transposons; particle-based methods, such as using a gene gun, magnectofection or magnet assisted transfection, particle bombardment; and hybrid methods, such as nucleofection.

The term “transfected” or “transformed” or “transduced” as used herein refers to a process by which exogenous nucleic acid is transferred or introduced into the host cell. A “transfected” or “transformed” or “transduced” cell is one, which has been transfected, transformed or transduced with exogenous nucleic acid.

The term “in vivo” refers to inside the body of the organism from which the cell is obtained. “Ex vivo” or “in vitro” means outside the body of the organism from which the cell is obtained.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, reducing recurrence rate of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

The term “effective amount” used herein refers to an amount of a compound or composition sufficient to treat a specified disorder, condition or disease such as ameliorate, palliate, lessen, and/or delay one or more of its symptoms. As is understood in the art, an “effective amount” may be in one or more doses, i.e., a single dose or multiple doses may be required to achieve the desired treatment endpoint.

A “subject,” an “individual,” or a “patient” are used herein interchangeably for purposes of treatment, and refers to any animal classified as a mammal, including humans, domestic and farm animals, and zoo, sports, or pet animals, such as dogs, horses, cats, cows, etc. In some embodiments, the individual is a human individual.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X.”

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### Engineered CRISPR-Cas Systems

The present application provides split Cas12b polypeptides, engineered guide RNAs and engineered CRISPR-Cas systems comprising split Cas12b polypeptides. In some embodiments, the engineered CRISPR-Cas system comprises split Cas12b polypeptides and one or more guide RNAs.

Accordingly, in some embodiments, the present application provides an engineered Clustered Regularly Interspersed Short Palindromic Repeat (CRISPR)-CRISPR associated (Cas) (CRISPR-Cas) system comprising: (a) a first polypeptide comprising a N-terminal portion of a reference Cas12b protein, (b) a second polypeptide comprising a C-terminal portion of the reference Cas12b protein, and (c) a guide RNA comprising a guide sequence; wherein the reference Cas12b protein comprises from the N-terminus to the C-terminus: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III) and a second Nuc domain (Nuc-II), wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; and wherein the first polypeptide, the second polypeptide and the guide RNA are capable of associating with each other to form a CRIPSR complex that specifically binds to a target nucleic acid comprising a target sequence complementary to the guide sequence. In some embodiments, the reference Cas12b protein is a Cas12b protein from Alicyclobacillus acidiphilus (AaCas12b), Bh3Cas12b or TcCas12b or a functional derivative thereof. In some embodiments, the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein. In some embodiments, the reference Cas12b protein is enzymatically active (e.g., a nuclease that cleaves a single strand or both strands of a duplex nucleic acid). In some embodiments, the reference Cas12b protein is enzymatically inactive.

In some embodiments, there is provided an engineered CRISPR-Cas system comprising: (a) a first polypeptide comprising a N-terminal portion of a reference Cas12b protein and a first functional domain, (b) a second polypeptide comprising a C-terminal portion of the reference Cas12b protein and a second functional domain, and (c) a guide RNA comprising a guide sequence; wherein the reference Cas12b protein comprises from the N-terminus to the C-terminus: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III) and a second Nuc domain (Nuc-II), wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; wherein the reference Cas12b protein is enzymatically inactive; and wherein the first polypeptide, the second polypeptide and the guide RNA are capable of associating with each other to form a CRIPSR complex that specifically binds to a target nucleic acid comprising a target sequence complementary to the guide sequence. In some embodiments, the reference Cas12b protein is a Cas12b protein from Alicyclobacillus acidiphilus (AaCas12b), Bh3Cas12b or TcCas12b or a functional derivative thereof. In some embodiments, the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein. In some embodiments, the first functional domain and/or the second functional domain is selected from the group consisting of a translation initiator domain, a transcription repressor domain, a transactivation domain, an epigenetic modification domain, and a nuclease domain.

In some embodiments, there is provided an engineered CRISPR-Cas system comprising: (a) a first polypeptide comprising a N-terminal portion of a reference Cas12b protein and a first dimerization domain, (b) a second polypeptide comprising a C-terminal portion of the reference Cas12b protein and a second dimerization domain, and (c) a guide RNA comprising a guide sequence; wherein the reference Cas12b protein comprises from the N-terminus to the C-terminus: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III) and a second Nuc domain (Nuc-II), wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; wherein the first polypeptide, the second polypeptide and the guide RNA are capable of associating with each other in the presence of an inducer to form a CRIPSR complex that specifically binds to a target nucleic acid comprising a target sequence complementary to the guide sequence. In some embodiments, the reference Cas12b protein is a Cas12b protein from Alicyclobacillus acidiphilus (AaCas12b), Bh3Cas12b or TcCas12b or a functional derivative thereof. In some embodiments, the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein. In some embodiments, the reference Cas12b protein is enzymatically active (e.g., a nuclease that cleaves a single strand or both strands of a duplex nucleic acid). In some embodiments, the reference Cas12b protein is enzymatically inactive. In some embodiments, the first polypeptide further comprises a functional domain fused to the N-terminal portion of the reference Cas12b protein, and/or the second polypeptide further comprises a functional domain fused to the C-terminal portion of the reference Cas12b protein. In some embodiments, the functional domain is selected from the group consisting of a translation initiator domain, a transcription repressor domain, a transactivation domain, an epigenetic modification domain, and a nuclease domain.

In some embodiments, there is provided an engineered CRISPR-Cas system comprising: (a) a first polypeptide comprising a N-terminal portion of a reference Cas12b protein and a first dimerization domain, (b) a second polypeptide comprising a C-terminal portion of the reference Cas12b protein and a second dimerization domain, and (c) a guide RNA comprising a guide sequence; wherein the reference Cas12b protein comprises from the N-terminus to the C-terminus: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III) and a second Nuc domain (Nuc-II), wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; wherein the first polypeptide, the second polypeptide and the guide RNA are capable of associating with each other in the absence of any inducer to form a CRIPSR complex that specifically binds to a target nucleic acid comprising a target sequence complementary to the guide sequence. In some embodiments, the reference Cas12b protein is a Cas12b protein from Alicyclobacillus acidiphilus (AaCas12b), Bh3Cas12b or TcCas12b or a functional derivative thereof. In some embodiments, the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein. In some embodiments, the reference Cas12b protein is enzymatically active (e.g., a nuclease that cleaves a single strand or both strands of a duplex nucleic acid). In some embodiments, the reference Cas12b protein is enzymatically inactive. In some embodiments, the first polypeptide further comprises a functional domain fused to the N-terminal portion of the reference Cas12b protein, and/or the second polypeptide further comprises a functional domain fused to the C-terminal portion of the reference Cas12b protein. In some embodiments, the functional domain is selected from the group consisting of a translation initiator domain, a transcription repressor domain, a transactivation domain, an epigenetic modification domain, and a nuclease domain.

In some embodiments, there is provided an engineered CRISPR-Cas system comprising: (a) a first polypeptide comprising a N-terminal portion of a reference Cas12b protein, (b) a second polypeptide comprising a C-terminal portion of the reference Cas12b protein, and (c) a guide RNA comprising a guide sequence; wherein the reference Cas12b protein comprises from the N-terminus to the C-terminus: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III) and a second Nuc domain (Nuc-II), wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; wherein the first polypeptide and the second polypeptide do not comprise dimerization domains; and wherein the first polypeptide, the second polypeptide and the guide RNA are capable of associating with each other to form a CRIPSR complex that specifically binds to a target nucleic acid comprising a target sequence complementary to the guide sequence. In some embodiments, the reference Cas12b protein is a Cas12b protein from Alicyclobacillus acidiphilus (AaCas12b), Bh3Cas12b or TcCas12b or a functional derivative thereof. In some embodiments, the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein. In some embodiments, the reference Cas12b protein is enzymatically active (e.g., a nuclease that cleaves a single strand or both strands of a duplex nucleic acid). In some embodiments, the reference Cas12b protein is enzymatically inactive. In some embodiments, the first polypeptide further comprises a functional domain fused to the N-terminal portion of the reference Cas12b protein, and/or the second polypeptide further comprises a functional domain fused to the C-terminal portion of the reference Cas12b protein. In some embodiments, the functional domain is selected from the group consisting of a translation initiator domain, a transcription repressor domain, a transactivation domain, an epigenetic modification domain, and a nuclease domain.

In some embodiments, there is provided an engineered CRISPR-Cas system comprising: (a) a first polypeptide comprising a N-terminal portion of a reference Cas12b protein and a first dimerization domain, (b) a second polypeptide comprising a C-terminal portion of the reference Cas12b protein and a second dimerization domain, and (c) a guide RNA comprising a guide sequence, wherein the guide RNA is a truncated sgRNA comprising a tracrRNA sequence and a crRNA sequence comprising the guide sequence, and wherein compared to a full-length sgRNA comprising a wildtype tracrRNA sequence and a wildtype crRNA sequence corresponding to the reference Cas12b protein, the truncated sgRNAs lacks one or more stem loops; wherein the reference Cas12b protein comprises from the N-terminus to the C-terminus: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III) and a second Nuc domain (Nuc-II), wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; wherein the second polypeptide and the guide RNA are capable of associating with each other in the presence of an inducer to form a CRIPSR complex that specifically binds to a target nucleic acid comprising a target sequence complementary to the guide sequence. In some embodiments, the reference Cas12b protein is a Cas12b protein from Alicyclobacillus acidiphilus (AaCas12b), Bh3Cas12b or TcCas12b or a functional derivative thereof. In some embodiments, the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein. In some embodiments, the reference Cas12b protein is enzymatically active (e.g., a nuclease that cleaves a single strand or both strands of a duplex nucleic acid). In some embodiments, the reference Cas12b protein is enzymatically inactive. In some embodiments, the first polypeptide further comprises a functional domain fused to the N-terminal portion of the reference Cas12b protein, and/or the second polypeptide further comprises a functional domain fused to the C-terminal portion of the reference Cas12b protein. In some embodiments, the functional domain is selected from the group consisting of a translation initiator domain, a transcription repressor domain, a transactivation domain, an epigenetic modification domain, and a nuclease domain.

In some embodiments, the present application provides an engineered Clustered Regularly Interspersed Short Palindromic Repeat (CRISPR)-CRISPR associated (Cas) (CRISPR-Cas) system comprising: (a) a first polypeptide comprising a N-terminal portion of a reference Cas12b protein, (b) a second polypeptide comprising a C-terminal portion of the reference Cas12b protein, and (c) a guide RNA comprising a guide sequence; wherein the reference Cas12b protein comprises from the N-terminus to the C-terminus: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III) and a second Nuc domain (Nuc-II), wherein the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein; and wherein the first polypeptide, the second polypeptide and the guide RNA are capable of associating with each other to form a CRIPSR complex that specifically binds to a target nucleic acid comprising a target sequence complementary to the guide sequence. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 3, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 4. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 83, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 84. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 86, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 87. In some embodiments, the first polypeptide and the second polypeptide do not comprise dimerization domains. In some embodiments, the reference Cas12b protein is a Cas12b protein from Alicyclobacillus acidiphilus (AaCas12b), Bh3Cas12b or TcCas12b or a functional derivative thereof. In some embodiments, the reference Cas12b protein is enzymatically active (e.g., a nuclease that cleaves a single strand or both strands of a duplex nucleic acid). In some embodiments, the reference Cas12b protein is enzymatically inactive. In some embodiments, the first polypeptide further comprises a functional domain fused to the N-terminal portion of the reference Cas12b protein, and/or the second polypeptide further comprises a functional domain fused to the C-terminal portion of the reference Cas12b protein. In some embodiments, the functional domain is selected from the group consisting of a translation initiator domain, a transcription repressor domain, a transactivation domain, an epigenetic modification domain, and a nuclease domain.

In some embodiments, there is provided an engineered CRISPR-Cas system comprising: (a) a first polypeptide comprising a N-terminal portion of a reference Cas12b protein and a first dimerization domain, (b) a second polypeptide comprising a C-terminal portion of the reference Cas12b protein and a second dimerization domain, and (c) a guide RNA comprising a guide sequence; wherein the reference Cas12b protein comprises from the N-terminus to the C-terminus: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III) and a second Nuc domain (Nuc-II), wherein the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein; and wherein the first polypeptide, the second polypeptide and the guide RNA are capable of associating with each other in the presence of an inducer to form a CRIPSR complex that specifically binds to a target nucleic acid comprising a target sequence complementary to the guide sequence. In some embodiments, the first dimerization domain is a FKBP domain and the second dimerization domain is a FRB domain; or the first dimerization domain is a FRB domain and the second dimerization domain is a FKBP domain. In some embodiments, the inducer is rapamycin. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 3, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 4. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 83, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 84. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 86, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 87. In some embodiments, the first polypeptide comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 13, and wherein the second polypeptide comprises an amino acid sequence having at least about 85% (e.g., at least about any one of 90%, 91%, 92%, 93%, 94%, 95%, 96%, 97%, 98%, or 99%) sequence identity, or 100% sequence identity to the amino acid sequence of SEQ ID NO: 14. In some embodiments, the reference Cas12b protein is a Cas12b protein from Alicyclobacillus acidiphilus (AaCas12b), Bh3Cas12b or TcCas12b or a functional derivative thereof. In some embodiments, the reference Cas12b protein is enzymatically active (e.g., a nuclease that cleaves a single strand or both strands of a duplex nucleic acid). In some embodiments, the reference Cas12b protein is enzymatically inactive. In some embodiments, the first polypeptide further comprises a functional domain fused to the N-terminal portion of the reference Cas12b protein, and/or the second polypeptide further comprises a functional domain fused to the C-terminal portion of the reference Cas12b protein. In some embodiments, the functional domain is selected from the group consisting of a translation initiator domain, a transcription repressor domain, a transactivation domain, an epigenetic modification domain, and a nuclease domain.

In some embodiments according to any one of the engineered CRISPR-Cas systems described herein, wherein the reference Cas12b protein has N amino acid residues; the first polypeptide comprises amino acid residues 1 to X of the reference Cas12b protein, wherein X is an integer greater than 1 and smaller than N; and the second polypeptide comprises amino acid residues X+1 to N of the reference Cas12b protein.

In some embodiments according to any one of the engineered CRISPR-Cas systems described herein, the engineered CRISPR-Cas system comprises a crRNA molecule and a tracrRNA molecule. In some embodiments, the engineered CRISPR-Cas system comprises a single guide RNA (sgRNA) comprising a crRNA sequence fused to a tracrRNA sequence. In some embodiments, the engineered CRISPR-Cas system comprises a guide RNA comprising a plurality of crRNA sequences, wherein each crRNA sequences comprises a different target sequence.

### Split Cas12b Polypeptides

The CRISPR-Cas systems described herein may comprise any pair of polypeptides (also referred herein as “split Cas12b polypeptides”) comprising split Cas12b portions in this section. In some embodiments, the CRISPR-Cas system comprises: (a) a first polypeptide comprising a N-terminal portion of a reference Cas12b protein, and (b) a second polypeptide comprising a C-terminal portion of the reference Cas12b protein, wherein the first polypeptide and the second polypeptide are capable of associating with each other in the presence of a guide RNA comprising a guide sequence to form a CRIPSR complex that specifically binds to a target nucleic acid comprising a target sequence complementary to the guide sequence.

The split Cas12b portions are designed based on Cas12b proteins that naturally occur in bacteria and archaea, or functional variants thereof. Recently, the type V-B CRISPR-Cas12b (also known as C2c1) system has been identified as a dual-RNA-guided DNA endonuclease system with distinct features from Cas9 and Cas12a (Shmakov, S. et al. Mol. Cell 60, 385-397 (2015)). First, Cas12b was reported to generate staggered ends distal to the protospacer adjacent motif (PAM) site in vitro when reconstituted with the crRNA/tracrRNA duplex. Second, although the RuvC domain of Cas12b is similar to that of Cas9 and Cas12a, its putative Nuc domain shares no sequence or structural similarity to the HNH domain of Cas9 and the Nuc domain of Cas12a. Moreover, Cas12b proteins are smaller than the most widely used SpCas9 and Cas12a (e.g., AacCas12b: 1,129 amino acids (aa); SpCas9: 1,369 aa; AsCas12a: 1,353aa; LbCas12a: 1,274 aa), making Cas12b suitable for adeno-associated virus (AAV)-mediated in vivo delivery in gene therapy. Compared with small-sized Cas9 proteins, such as SaCas9 and CjCas9, Cas12b recognizes simpler PAM sequences (e.g., AacCas12b: 5′-TTN-3′; compared to SaCas9: 5′-NNGRRT-3′ (SEQ ID NO: 28), CjCas9: 5′-NNNNRYAC-3′ (SEQ ID NO: 29)), which significantly increase the targeting range of Cas12b in the genome. Most importantly, Cas12b has minimal off-target effects and thus may serve as a safer choice for therapeutic and clinical applications.

Cas12b (C2c1) proteins from various organisms may be used to design split Cas12b portions of the present application. Exemplary Cas12b proteins have been described, for example, Shmakov, S. et al. Mol. Cell 60, 385-397 (2015); Shmakov, S. et al. Nat. Rev. Microbiol. 15, 169-182 (2017); Teng F. et al., Cell Discovery (2019) 5:23; WO2016205764, and WO2020/087631, which are incorporated herein by reference in their entirety.

In some embodiments, the split Cas12b portions are based on a reference Cas12b protein selected from Cas12b from Alicyclobacillus acidiphilus (AaCas12b), Cas12b from Alicyclobacillus kakegawensis (AkCas12b), Cas12b from Alicyclobacillus macrosporangiidus (AmCas12b), Cas12b from Bacillus hisashii (BhCas12b), BsCas12b from Bacillus, Cas12b from Bacillus sp. V3-13 (Bs3Cas12b), Cas12b from Desulfovibrio inopinatus (DiCas12b), Cas12b from Laceyella sediminis (LsCas12b), Cas12b from Spirochaetes bacterium (SbCas12b), Cas12b from Tuberibacillus calidus (TcCas12b) and functional derivatives thereof. In some embodiments, the reference Cas12b protein is a Cas12b protein from Alicyclobacillus acidiphilus (AaCas12b) or a functional derivative thereof.

In some embodiments, the split Cas12b portions are based on a reference Cas12b protein from Alicyclobacillus acidiphilus (AaCas12b) or a functional derivative thereof. Sequences of naturally occurring Cas12b proteins are known, for example, in UniProtKB ID: TOD7A2, which is incorporated herein by reference in its entirety. In some embodiments, the split Cas12b portions are based on a reference Cas12b protein comprising an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 33. In some embodiments, the split Cas12b portions are based on a reference Cas12b protein comprising the amino acid sequence of SEQ ID NO: 33.

In some embodiments, the split Cas12b portions are based on a reference Bs3Cas12b protein from Bacillus sp. V3-13 or a functional derivative thereof. In some embodiments, the split Cas12b portions are based on a reference Cas12b protein comprising an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 85. In some embodiments, the split Cas12b portions are based on a reference Cas12b protein comprising the amino acid sequence of SEQ ID NO: 85.

In some embodiments, the split Cas12b portions are based on a reference TcCas12b protein from Tuberibacillus calidus or a functional derivative thereof. In some embodiments, the split Cas12b portions are based on a reference Cas12b protein comprising an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 88. In some embodiments, the split Cas12b portions are based on a reference Cas12b protein comprising the amino acid sequence of SEQ ID NO: 88.

Alicyclobacillus acidoterrestris Cas12b amino acid sequence
SEQ ID NO: 30
MAVKSIKVKLRLDDMPEIRAGLWKLHKEVNAGVRYYTEWLSLLRQENLYRRSPNG
DGEQECDKTAEECKAELLERLRARQVENGHRGPAGSDDELLQLARQLYELLVPQAI
GAKGDAQQIARKFLSPLADKDAVGGLGIAKAGNKPRWVRMREAGEPGWEEEKEKA
ETRKSADRTADVLRALADFGLKPLMRVYTDSEMSSVEWKPLRKGQAVRTWDRDM
FQQAIERMMSWESWNQRVGQEYAKLVEQKNRFEQKNFVGQEHLVHLVNQLQQDM
KEASPGLESKEQTAHYVTGRALRGSDKVFEKWGKLAPDAPFDLYDAEIKNVQRRNT
RRFGSHDLFAKLAEPEYQALWREDASFLTRYAVYNSILRKLNHAKMFATFTLPDAT
AHPIWTRFDKLGGNLHQYTFLFNEFGERRHAIRFHKLLKVENGVAREVDDVTVPISM
SEQLDNLLPRDPNEPIALYFRDYGAEQHFTGEFGGAKIQCRRDQLAHMHRRRGARD
VYLNVSVRVQSQSEARGERRPPYAAVFRLVGDNHRAFVHFDKLSDYLAEHPDDGKL
GSEGLLSGLRVMSVDLGLRTSASISVFRVARKDELKPNSKGRVPFFFPIKGNDNLVAV
HERSQLLKLPGETESKDLRAIREERQRTLRQLRTQLAYLRLLVRCGSEDVGRRERSW
AKLIEQPVDAANHMTPDWREAFENELQKLKSLHGICSDKEWMDAVYESVRRVWRH
MGKQVRDWRKDVRSGERPKIRGYAKDVVGGNSIEQIEYLERQYKFLKSWSFFGKVS
GQVIRAEKGSRFAITLREHIDHAKEDRLKKLADRIIMEALGYVYALDERGKGKWVA
KYPPCQLILLEELSEYQFNNDRPPSENNQLMQWSHRGVFQELINQAQVHDLLVGTM
YAAFSSRFDARTGAPGIRCRRVPARCTQEHNPEPFPWWLNKFVVEHTLDACPLRAD
DLIPTGEGEIFVSPFSAEEGDFHQIHADLNAAQNLQQRLWSDFDISQIRLRCDWGEVD
GELVLIPRLTGKRTADSYSNKVFYTNTGVTYYERERGKKRRKVFAQEKLSEEEAELL
VEADEAREKSVVLMRDPSGIINRGNWTRQKEFWSMVNQRIEGYLVKQIRSRVPLQD
SACENTGDI
Alicyclobacillus acidiphilus Cas12b (AaCas12b) amino acid sequence
SEQ ID NO: 33
MAVKSMKVKLRLDNMPEIRAGLWKLHTEVNAGVRYYTEWLSLLRQENLYRRSPNG
DGEQECYKTAEECKAELLERLRARQVENGHCGPAGSDDELLQLARQLYELLVPQAIG
AKGDAQQIARKFLSPLADKDAVGGLGIAKAGNKPRWVRMREAGEPGWEEEKAKAE
ARKSTDRTADVLRALADFGLKPLMRVYTDSDMSSVQWKPLRKGQAVRTWDRDMFQ
QAIERMMSWESWNQRVGEAYAKLVEQKSRFEQKNFVGQEHLVQLVNQLQQDMKEA
SHGLESKEQTAHYLTGRALRGSDKVFEKWEKLDPDAPFDLYDTEIKNVQRRNTRRFG
SHDLFAKLAEPKYQALWREDASFLTRYAVYNSIVRKLNHAKMFATFTLPDATAHPIWT
RFDKLGGNLHQYTFLFNEFGEGRHAIRFQKLLTVEDGVAKEVDDVTVPISMSAQLDD
LLPRDPHELVALYFQDYGAEQHLAGEFGGAKIQYRRDQLNHLHARRGARDVYLNLS
VRVQSQSEARGERRPPYAAVFRLVGDNHRAFVHFDKLSDYLAEHPDDGKLGSEGLL
SGLRVMSVDLGLRTSASISVFRVARKDELKPNSEGRVPFCFPIEGNENLVAVHERSQL
LKLPGETESKDLRAIREERQRTLRQLRTQLAYLRLLVRCGSEDVGRRERSWAKLIEQP
MDANQMTPDWREAFEDELQKLKSLYGICGDREWTEAVYESVRRVWRHMGKQVRD
WRKDVRSGERPKIRGYQKDVVGGNSIEQIEYLERQYKFLKSWSFFGKVSGQVIRAEK
GSRFAITLREHIDHAKEDRLKKLADRIIMEALGYVYALDDERGKGKWVAKYPPCQLI
LLEELSEYQFNNDRPPSENNQLMQWSHRGVFQELLNQAQVHDLLVGTMYAAFSSRF
DARTGAPGIRCRRVPARCAREQNPEPFPWWLNKFVAEHKLDGCPLRADDLIPTGEGE
FFVSPFSAEEGDFHQIHADLNAAQNLQRRLWSDFDISQIRLRCDWGEVDGEPVLIPRT
TGKRTADSYGNKVFYTKTGVTYYERERGKKRRKVFAQEELSEEEAELLVEADEARE
KSVVLMRDPSGIINRGDWTRQKEFWSMVNQRIEGYLVKQIRSRVRLQESACENTGDI
Bs3 Cas12b
SEQ ID NO: 85
MAIRSIKLKMKTNSGTDSIYLRKALWRTHQLINEGIAYYMNLLTLYRQEAIGDKTKE
AYQAELINIIRNQQRNNGSSEEHGSDQEILALLRQLYELIIPSSIGESGDANQLGNKFLY
PLVDPNSQSGKGTSNAGRKPRWKRLKEEGNPDWELEKKKDEERKAKDPTVKIFDNL
NKYGLLPLFPLFTNIQKDIEWLPLGKRQSVRKWDKDMFIQAIERLLSWESWNRRVAD
EYKQLKEKTESYYKEHLTGGEEWIEKIRKFEKERNMELEKNAFAPNDGYFITSRQIRG
WDRVYEKWSKLPESASPEELWKVVAEQQNKMSEGFGDPKVFSFLANRENRDIWRG
HSERIYHIAAYNGLQKKLSRTKEQATFTLPDAIEHPLWIRYESPGGTNLNLFKLEEKQ
KKNYYVTLSKIIWPSEEKWIEKENIEIPLAPSIQFNRQIKLKQHVKGKQEISFSDYSSRIS
LDGVLGGSRIQFNRKYIKNHKELLGEGDIGPVFFNLVVDVAPLQETRNGRLQSPIGKA
LKVISSDFSKVIDYKPKELMDWMNTGSASNSFGVASLLEGMRVMSIDMGQRTSASV
SIFEVVKELPKDQEQKLFYSINDTELFAIHKRSFLLNLPGEVVTKNNKQQRQERRKKR
QFVRSQIRMLANVLRLETKKTPDERKKAIHKLMEIVQSYDSWTASQKEVWEKELNL
LTNMAAFNDEIWKESLVELHHRIEPYVGQIVSKWRKGLSEGRKNLAGISMWNIDELE
DTRRLLISWSKRSRTPGEANRIETDEPFGSSLLQHIQNVKDDRLKQMANLIIMTALGF
KYDKEEKDRYKRWKETYPACQIILFENLNRYLFNLDRSRRENSRLMKWAHRSIPRTV
SMQGEMFGLQVGDVRSEYSSRFHAKTGAPGIRCHALTEEDLKAGSNTLKRLIEDGFI
NESELAYLKKGDIIPSQGGELFVTLSKRYKKDSDNNELTVIHADINAAQNLQKRFWQ
QNSEVYRVPCQLARMGEDKLYIPKSQTETIKKYFGKGSFVKNNTEQEVYKWEKSEK
MKIKTDTTFDLQDLDGFEDISKTIELAQEQQKKYLTMFRDPSGYFFNNETWRPQKEY
WSIVNNIIKSCLKKKILSNKVEL
Tc Cas12b
SEQ ID NO: 88
MNIHLKELIRMATKSFILKMKTKNNPQLRLSLWKTHELFNFGVAYYMDLLSLFRQK
DLYMHNDEDPDHPVVLKKEEIQERLWMKVRETQQKNGFHGEVSKDEVLETLRALY
EELVPSAVGKSGEANQISNKYLYPLTDPASQSGKGTANSGRKPRWKKLKEAGDPSW
KDAYEKWEKERQEDPKLKILAALQSFGLIPLFRPFTENDHKAVISVKWMPKSKNQSV
RKFDKDMFNQAIERFLSWESWNEKVAEDYEKTVSIYESLQKELKGISTKAFEIMERV
EKAYEAHLREITFSNSTYRIGNRAIRGWTEIVKKWMKLDPSAPQGNYLDVVKDYQR
RHPRESGDFKLFELLSRPENQAAWREYPEFLPLYVKYRHAEQRMKTAKKQATFTLC
DPIRHPLWVRYEERSGTNLNKYRLIMNEKEKVVQFDRLICLNADGHYEEQEDVTVPL
APSQQFDDQIKFSSEDTGKGKHNFSYYHKGINYELKGTLGGARIQFDREHLLRRQGV
KAGNVGRIFLNVTLNIEPMQPFSRSGNLQTSVGKALKVYVDGYPKVVNFKPKELTEH
IKESEKNTLTLGVESLPTGLRVMSVDLGQRQAAAISIFEVVSEKPDDNKLFYPVKDTD
LFAVHRTSFNIKLPGEKRTERRMLEQQKRDQAIRDLSRKLKFLKNVLNMQKLEKTDE
REKRVNRWIKDREREEENPVYVQEFEMISKVLYSPHSVWVDQLKSIHRKLEEQLGKE
ISKWRQSISQGRQGVYGISLKNIEDIEKTRRLLFRWSMRPENPGEVKQLQPGERFAID
QQNHLNHLKDDRIKKLANQIVMTALGYRYDGKRKKWIAKHPACQLVLFEDLSRYA
FYDERSRLENRNLMRWSRREIPKQVAQIGGLYGLLVGEVGAQYSSRFHAKSGAPGIR
CRVVKEHELYITEGGQKVRNQKFLDSLVENNIIEPDDARRLEPGDLIRDQGGDKFATL
DERGELVITHADINAAQNLQKRFWTRTHGLYRIRCESREIKDAVVLVPSDKDQKEKM
ENLFGIGYLQPFKQENDVYKWVKGEKIKGKKTSSQSDDKELVSEILQEASVMADELK
GNRKTLFRDPSGYVFPKDRWYTGGRYFGTLEHLLKRKLAERRLFDGGSSRRGLFNG
TDSNTNVE

It is noted that orthologues having a certain sequence identity (e.g., at least about any one of 60%, 70%, 80%, 85%, 90%, 95%, 98% or higher) to Cas12b or fragments thereof may be used as basis to design the split Cas12b portions of the present application. The skilled artisan can determine, based on the purpose and application, the percentage of sequence identity of an orthologue of Cas12b or fragment thereof suitable for use in the present application. Methods for determining sequence identity values may be found in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Various Cas12b orthologues have been described in WO2020/087631 and Teng F. et al., Cell Discovery (2019) 5:23, which are incorporated herein by reference in their entirety.

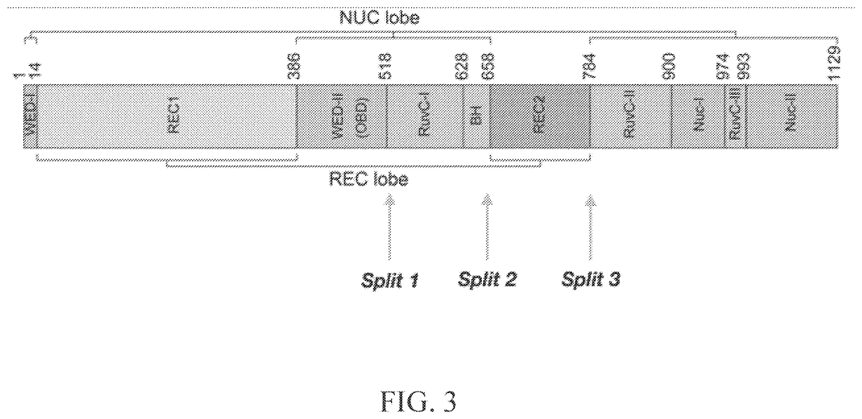

Naturally occurring Cas12b proteins have various structural domains, for example, as shown in FIG. 3. In some embodiments, a reference Cas12b protein comprises from the N-terminus to the C-terminus: a first WED domain (WED-I; also known as OBD-I domain), a first REC domain (REC1), a second WED domain (WED-II; also known as OBD-II domain), a first RuvC domain (RuvC-I), a bridge helix (BH) domain, a second RuvC domain (RuvC-II), a first Nuc domain (Nuc-I; also known as UK-I domain), a third RuvC domain (RuvC-III) and a second Nuc domain (Nuc-II; also known as UK-II domain). Domain boundaries may be determined using known methods in the art, such as based on crystal structures of a reference Cas12b protein (e.g., PDB ID Nos: 5U30, 5U31, 5U33, 5U34 and 5WQE for AaCas12b), and/or sequence homology to known functional domains in a reference Cas12b protein. In some embodiments, the AaCas12b has the following domains: WEB-I domain (amino acid residues 1-14), REC1 domain (amino acid residues 15-386), WED-II domain (amino acid residues 387-518), RuvC-I domain (amino acid residues 519-628), BH domain (amino acid residues 629-658), REC2 domain (amino acid residues 659-783), RuvC-II domain (amino acid residues 784-900), Nuc-I domain (amino acid residues 901-974), RuvC-III domain (amino acid residues 975-993), and Nuc-II domain (amino acid residues 994-1129), wherein the amino acid numbering is based on SEQ ID NO: 33.

Crystal structures of Alicyclobacillus acidoterrestris Cas12b bound to sgRNA as a binary complex and to target DNAs as ternary complexes have been described in Yang H., et al. Cell 167:1814-1828(2016) and Liu L. et al. Mol. Cell 65:310-322(2017). Briefly, the crystal structures show 2 discontinuous REC (recognition, residues 15-386, 658-783) and NUC (nuclease, residues 1-14, 387-658 and 784-1129) lobes composed of several domains each, which are shown in FIG. 3 with the domain boundaries annotated according to Liu et al. Yang et al. has slightly different domain boundaries, which differ only from those in Liu et al. by a few amino acid residues. The crRNA (or single guide RNA, sgRNA) binds in a central channel between the two lobes. PAM recognition is sequence specific and occurs mostly via interaction with the REC1 (helical-1) and WED-II (OBD-II) domains. The sgRNA-target DNA heteroduplex binds primarily to the REC lobe in a sequence-independent manner.

It is understood that other Cas12b orthologues, such as BhCas12b, Bs3Cas12b, LsCas12b, SbCas12b, AkCas12b, AmCas12b, BsCas12b, and DiCas12b etc., have similar domain structures as AaCas12b and other exemplary reference Cas12b proteins described herein, and split Cas12b portions may be designed based on any one of the orthologues using split positions that correspond to the exemplary AaCas12b, Bs3Cas12b and TcCas12b split portions described herein. Corresponding positions refer to the positions in two polypeptides that are aligned with each other when the amino acid sequences of the two polypeptides are aligned with each other. For example, FIG. S2 of Teng F. et al., Cell Discovery (2019) 5:23 provides an alignment of AaCas12b, AkCas12b, AmCas12b, Bs3Cas12b, BsCas12b, LsCas12b, BhCas12b and SbCas12b, which is incorporated herein by reference.

In some embodiments, the split Cas12b portions are based on a functional variant of a naturally occurring Cas12b protein. In some embodiments, the functional variant has one or more mutations, such as amino acid substitutions, insertions and deletions. By way of example, the functional variant may comprise any one of 1, 2, 3, 4, 5, 6, 7, 8, 9, 10 or more amino acid substitutions compared to a wild type naturally occurring Cas12b protein. In some embodiments, the one or more substitutions are conservative substitutions. In some embodiments, the functional variant has all domains of a naturally occurring Cas12b protein. In some embodiments, the functional variant does not have one or more domains of a naturally occurring Cas12b protein.

In some embodiments, the reference Cas12b protein is enzymatically active. In some embodiments, the reference Cas12b is a nuclease that cleaves both strands of a target duplex nucleic acid (e.g., duplex DNA). In some embodiments, the reference Cas12b is a nickase, i.e., cleaving a single strand of a target duplex nucleic acid (e.g., duplex DNA). In some embodiments, the reference Cas12b protein is enzymatically inactive.

The reference Cas12b is split in the sense that the two split Cas12b portions substantially comprise a functional Cas12b. That Cas12b may function as a genome editing enzyme (when forming a complex with a target DNA and a guide RNA), such as a nuclease that cleaves a single strand or both strands of a duplex nucleic acid, or it may be a catalytically dead-Cas12b (dCas12b), which is essentially a DNA-binding protein with very little or no catalytic activity, due to typically mutation(s) in its catalytic domains. Mutations at one or more amino acid residues in the active site of a reference Cas12b can result in a catalytically dead Cas12b. For example, R785A, R911A, or D977A mutants of AaCas12b have no nuclease activities in human cells. See, for example, Teng F. et al., Cell Discovery, 4, Article number: 63 (2018), which is incorporated herein by reference in its entirety. D570A AaCas12b is also known to have no nuclease activities. Corresponding mutations in homologues and orthologues of AaCas12b are also contemplated herein. In some embodiments, the reference Cas12b is AaCas12b (D570A). In some embodiments, the reference Cas12b is AaCas12b (R785A). In some embodiments, the reference Cas12b is AaCas12b (R911A). In some embodiments, the reference Cas12b is AaCas12b (D977A). In some embodiments, the reference Cas12b is BthCas12b (D573A). In some embodiments, the split Cas12b portions are based on a catalytically dead Cas12b protein comprising an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 33, 85 or 85, and having one or more of D570A, R785A, R911A and/or D977A mutations, wherein the amino acid numbering is according to SEQ ID NO: 33.

The split Cas12b portions described herein can be designed by dividing (i.e., splitting) a reference Cas12b protein (e.g., a full-length Cas12b protein or a functional variant thereof) into two halves at a split position, which is the point at which the N-terminal portion of the reference Cas12b protein is separated from the C-terminal portion. In some embodiments, the N-terminal portion comprises amino acid residues 1 to X, whilst the C-terminal portion comprises amino acid residues X+1 to the C-terminus end of the reference Cas12b protein. In this example, the numbering is contiguous, but this may not always be necessary as amino acids (or the nucleotides encoding them) could be trimmed from the end of either one of the split ends, and/or mutations (e.g., insertions, deletions and substitutions) at internal regions of the polypeptide chain(s) are also contemplated, provided that sufficient DNA binding activity and, if required, DNA nickase or cleavage activity, of the reconstituted Cas12b protein is retained, for example at least 40%, 50%, 60%, 70%, 80%, 90% or 95% activity compared to the reference Cas12b protein.

For example, FIG. 3 shows exemplary splitting positions for three pairs of split Cas12b polypeptides, in which the amino acid residue numbering corresponds to that of a wild-type AaCas12b protein, e.g., as in SEQ ID NO: 33. However, it is envisaged that functional variants, including mutants of wildtype Cas12b proteins, can be used as basis for designing the split Cas12b polypeptides. The numbering may also not follow exactly the reference Cas12b numbering as, for instance, some N- or C-terminal truncations or deletions, as well as internal mutations to a wildtype Cas12b protein may be used. A skilled person in the art could readily use the information of the exemplary split Cas12b polypeptides described herein to design counterpart split Cas12b polypeptides based on other Cas12b proteins and functional variants, e.g., by using standard sequence alignment tools.

A skilled artisan would recognize that variations of the exemplary splitting schemes as illustrated in FIG. 3 can be adopted to provide alternative split Cas12b portions that are encompassed by the present application. The exact split position may be selected in the vicinity of the splitting positions in FIG. 3 based on crystal structure data and/or computational structure predictions. For example, the split position may be located within a flexible region, such as a loop. Preferably, the split position occurs where an interruption of the amino acid sequence does not result in the partial or full destruction of a structural feature (e.g., alpha-helixes or beta-sheets). Unstructured regions (regions that do not show up in the crystal structure because these regions are not structured enough to be “frozen” in a crystal) are often preferred options. It is contemplated that the splits can be made in unstructured regions that are exposed on the surface of a reference Cas12b protein.

In some embodiments, the reference Cas12b protein is not split at or in the vicinity (e.g., within about 10, 8, 6, 5, 4, 3, 2, or 1 amino acid residues) to an amino acid residue involved in interaction with a guide RNA, and/or a target RNA. For example, amino acid residues 4-9, 118-122, 143-144, 442-446, 573-574, 742-746, 753-754, 792-796, 800-819, 835-839, 897-900 and 973-978 of the AaCas12b protein are involved in interaction with a single-guide RNA and/or a target DNA, wherein the numbering is based on SEQ ID NO: 33.

In some embodiments, the N-terminal portion of the reference Cas12b protein comprises the WED-I domain of the reference Cas12b protein, and the C-terminal portion of the reference Cas12b protein comprises the REC1, WED-II, RuvC-I, BH, REC2, RuvC-II, Nuc-I, RuvC-III and Nuc-II domains of the reference Cas12b protein.

In some embodiments, the N-terminal portion of the reference Cas12b protein comprises the WED-I, and REC1 domains of the reference Cas12b protein, and the C-terminal portion of the reference Cas12b protein comprises the WED-II, RuvC-I, BH, REC2, RuvC-II, Nuc-I, RuvC-III and Nuc-II domains of the reference Cas12b protein.

In some embodiments, the N-terminal portion of the reference Cas12b protein comprises the WED-I, REC1, and WED-II domains of the reference Cas12b protein, and the C-terminal portion of the reference Cas12b protein comprises the RuvC-I, BH, REC2, RuvC-II, Nuc-I, RuvC-III and Nuc-II domains of the reference Cas12b protein.

In some embodiments, the N-terminal portion of the reference Cas12b protein comprises the WED-I, REC1, WED-II, and RuvC-I domains of the reference Cas12b protein, and the C-terminal portion of the reference Cas12b protein comprises the BH, REC2, RuvC-II, Nuc-I, RuvC-III and Nuc-II domains of the reference Cas12b protein.

In some embodiments, the N-terminal portion of the reference Cas12b protein comprises the WED-I, REC1, WED-II, RuvC-I, and BH domains of the reference Cas12b protein, and the C-terminal portion of the reference Cas12b protein comprises the REC2, RuvC-II, Nuc-I, RuvC-III and Nuc-II domains of the reference Cas12b protein.

In some embodiments, the N-terminal portion of the reference Cas12b protein comprises the WED-I, REC1, WED-II, RuvC-I, BH, and REC2 domains of the reference Cas12b protein, and the C-terminal portion of the reference Cas12b protein comprises the RuvC-II, Nuc-I, RuvC-III and Nuc-II domains of the reference Cas12b protein.

In some embodiments, the N-terminal portion of the reference Cas12b protein comprises the WED-I, REC1, WED-II, RuvC-I, BH, REC2, and RuvC-II domains of the reference Cas12b protein, and the C-terminal portion of the reference Cas12b protein comprises the Nuc-I, RuvC-III and Nuc-II domains of the reference Cas12b protein.

In some embodiments, the N-terminal portion of the reference Cas12b protein comprises the WED-I, REC1, WED-II, RuvC-I, BH, REC2, RuvC-II, and Nuc-I domains of the reference Cas12b protein, and the C-terminal portion of the reference Cas12b protein comprises the RuvC-III and Nuc-II domains of the reference Cas12b protein.

In some embodiments, the N-terminal portion of the reference Cas12b protein comprises the WED-I, REC1, WED-II, RuvC-I, BH, REC2, RuvC-II, Nuc-I, and RuvC-III domains of the reference Cas12b protein, and the C-terminal portion of the reference Cas12b protein comprises the Nuc-II domain of the reference Cas12b protein.

In some embodiments, the RuvC-I, BH, and REC2 domains of the reference Cas12b protein are split between the N-terminal portion of the reference Cas12b protein and the C-terminal portion of the reference Cas12b protein, i.e., the split position is within RuvC-I, BH, or REC2 domain, or between RuvC-I and BH domains, or between BH and REC2 domains.

In some embodiments, the reference Cas12b protein is split at an amino acid residue within amino acid residues corresponding to amino acid residues 516 to 793 of the AaCas12b protein, wherein the numbering is based on SEQ ID NO: 33. In some embodiments, the reference Cas12b protein is split at an amino acid residue bordering the WED-II domain and the RuvC-I domain. In some embodiments, the reference Cas12b protein is split at an amino acid residue within amino acid residues corresponding to amino acid residues 516 to 519 of the AaCas12b protein, wherein the numbering is based on SEQ ID NO: 33. In some embodiments, the reference Cas12b protein is split at an amino acid residue bordering the BH domain and the REC2 domain. In some embodiments, the reference Cas12b protein is split at an amino acid residue within amino acid residues corresponding to amino acid residues 621 to 627 of the AaCas12b protein, wherein the numbering is based on SEQ ID NO: 33. In some embodiments, the reference Cas12b protein is split at an amino acid residue bordering the REC2 domain and the RuvC-II domain. In some embodiments, the reference Cas12b protein is split at an amino acid residue within amino acid residues corresponding to amino acid residues 777 to 793 of the AaCas12b protein, wherein the numbering is based on SEQ ID NO: 33. In some embodiments, the reference Cas12b protein is split within the RCE2 domain. In some embodiments, the reference Cas12b protein is split at an amino acid residue within amino acid residues corresponding to amino acid residues 659 to 664, 676 to 684, or 702 to 706 of the AaCas12b protein, wherein the numbering is based on SEQ ID NO: 33.

In some embodiments, the reference Cas12b protein is split at an amino acid residue within no more than about 20 (e.g., no more than about any one of 18, 16, 14, 12, 10, 8, 6, 5, 4, 3, 2, or 1) amino acid residues from an amino acid residue that corresponds to amino acid residue 518 of the AaCas12b protein, wherein the numbering is based on SEQ ID NO: 33. In some embodiments, the reference Cas12b protein is split at an amino acid residue that corresponds to amino acid residue 518 of the AaCas12b protein, wherein the numbering is based on SEQ ID NO: 33. In some embodiments, the reference Cas12b protein is split at an amino acid residue within no more than about 20 (e.g., no more than about any one of 18, 16, 14, 12, 10, 8, 6, 5, 4, 3, 2, or 1) amino acid residues from an amino acid residue that corresponds to amino acid residue 658 of the AaCas12b protein, wherein the numbering is based on SEQ ID NO: 33. In some embodiments, the reference Cas12b protein is split at an amino acid residue that corresponds to amino acid residue 658 of the AaCas12b protein, wherein the numbering is based on SEQ ID NO: 33. In some embodiments, the reference Cas12b protein is split at an amino acid residue within no more than about 20 (e.g., no more than about any one of 18, 16, 14, 12, 10, 8, 6, 5, 4, 3, 2, or 1) amino acid residues from an amino acid residue that corresponds to amino acid residue 783 of the AaCas12b protein, wherein the numbering is based on SEQ ID NO: 33. In some embodiments, the reference Cas12b protein is split at an amino acid residue that corresponds to amino acid residue 783 of the AaCas12b protein, wherein the numbering is based on SEQ ID NO: 33.

In some embodiments, the N-terminal portion of the reference Cas12b protein comprises the WED-I, REC1, WED-II, RuvC-I and BH domains of an AaCas12b protein, and wherein the C-terminal portion of the reference Cas12b protein comprises the REC2, RuvC-II, Nuc-I, RuvC-III and Nuc-II domains of the AaCas12b protein. In some embodiments, the N-terminal portion of the reference Cas12b protein comprises amino acid residues 1 to 658 of the reference Cas12b protein, and the C-terminal portion of the reference Cas12b protein comprises amino acid residues 659 to 1129 of the reference Cas12b protein, wherein the amino acid residue numbering is according to SEQ ID NO: 33. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 3, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 4. In some embodiments, the N-terminal portion of the reference Cas12b protein comprises the amino acid sequence of SEQ ID NO: 3, and wherein the C-terminal portion of the reference Cas12b protein comprises the amino acid sequence of SEQ ID NO: 4.

AaCas12b NT2₁₋₆₅₈
SEQ ID NO: 3
MAVKSMKVKLRLDNMPEIRAGLWKLHTEVNAGVRYYTEWLSLLRQENLYRRSPNG
DGEQECYKTAEECKAELLERLRARQVENGHCGPAGSDDELLQLARQLYELLVPQAI
GAKGDAQQIARKFLSPLADKDAVGGLGIAKAGNKPRWVRMREAGEPGWEEEKAKA
EARKSTDRTADVLRALADFGLKPLMRVYTDSDMSSVQWKPLRKGQAVRTWDRDM
FQQAIERMMSWESWNQRVGEAYAKLVEQKSRFEQKNFVGQEHLVQLVNQLQQDM
KEASHGLESKEQTAHYLTGRALRGSDKVFEKWEKLDPDAPFDLYDTEIKNVQRRNT
RRFGSHDLFAKLAEPKYQALWREDASFLTRYAVYNSIVRKLNHAKMFATFTLPDAT
AHPIWTRFDKLGGNLHQYTFLFNEFGEGRHAIRFQKLLTVEDGVAKEVDDVTVPISM
SAQLDDLLPRDPHELVALYFQDYGAEQHLAGEFGGAKIQYRRDQLNHLHARRGAR
DVYLNLSVRVQSQSEARGERRPPYAAVFRLVGDNHRAFVHFDKLSDYLAEHPDDGK
LGSEGLLSGLRVMSVDLGLRTSASISVFRVARKDELKPNSEGRVPFCFPIEGNENLVA
VHERSQLLKLPGETESKDLRAIREERQRTLRQLRTQLAYLRLLVRC
AaCas12b CT2₆₅₉₋₁₁₂₉
SEQ ID NO: 4
GSEDVGRRERSWAKLIEQPMDANQMTPDWREAFEDELQKLKSLYGICGDREWTEA
VYESVRRVWRHMGKQVRDWRKDVRSGERPKIRGYQKDVVGGNSIEQIEYLERQYK
FLKSWSFFGKVSGQVIRAEKGSRFAITLREHIDHAKEDRLKKLADRIIMEALGYVYAL
DDERGKGKWVAKYPPCQLILLEELSEYQFNNDRPPSENNQLMQWSHRGVFQELLNQ
AQVHDLLVGTMYAAFSSRFDARTGAPGIRCRRVPARCAREQNPEPFPWWLNKFVAE
HKLDGCPLRADDLIPTGEGEFFVSPFSAEEGDFHQIHADLNAAQNLQRRLWSDFDISQ
IRLRCDWGEVDGEPVLIPRTTGKRTADSYGNKVFYTKTGVTYYERERGKKRRKVFA
QEELSEEEAELLVEADEAREKSVVLMRDPSGIINRGDWTRQKEFWSMVNQRIEGYLV
KQIRSRVRLQESACENTGDI

In some embodiments, the N-terminal portion of the reference Cas12b protein comprises the WED-I, REC1, WED-II, RuvC-I and BH domains of a Bs3Cas12b protein, and wherein the C-terminal portion of the reference Cas12b protein comprises the REC2, RuvC-II, Nuc-I, RuvC-III and Nuc-II domains of the Bs3Cas12b protein. In some embodiments, the N-terminal portion of the reference Cas12b protein comprises amino acid residues 1 to 650 of the reference Cas12b protein, and the C-terminal portion of the reference Cas12b protein comprises amino acid residues 651 to 1112 of the reference Cas12b protein, wherein the amino acid residue numbering is according to SEQ ID NO: 85. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 83, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 84. In some embodiments, the N-terminal portion of the reference Cas12b protein comprises the amino acid sequence of SEQ ID NO: 83, and wherein the C-terminal portion of the reference Cas12b protein comprises the amino acid sequence of SEQ ID NO: 84.

Bs3 Cas12b NT₁₋₆₅₀
SEQ ID NO: 83
MAIRSIKLKMKTNSGTDSIYLRKALWRTHQLINEGIAYYMNLLTLYRQEAIGDKTKE
AYQAELINIIRNQQRNNGSSEEHGSDQEILALLRQLYELIIPSSIGESGDANQLGNKFLY
PLVDPNSQSGKGTSNAGRKPRWKRLKEEGNPDWELEKKKDEERKAKDPTVKIFDNL
NKYGLLPLFPLFTNIQKDIEWLPLGKRQSVRKWDKDMFIQAIERLLSWESWNRRVAD
EYKQLKEKTESYYKEHLTGGEEWIEKIRKFEKERNMELEKNAFAPNDGYFITSRQIRG
WDRVYEKWSKLPESASPEELWKVVAEQQNKMSEGFGDPKVFSFLANRENRDIWRG
HSERIYHIAAYNGLQKKLSRTKEQATFTLPDAIEHPLWIRYESPGGTNLNLFKLEEKQ
KKNYYVTLSKIIWPSEEKWIEKENIEIPLAPSIQFNRQIKLKQHVKGKQEISFSDYSSRIS
LDGVLGGSRIQFNRKYIKNHKELLGEGDIGPVFFNLVVDVAPLQETRNGRLQSPIGKA
LKVISSDFSKVIDYKPKELMDWMNTGSASNSFGVASLLEGMRVMSIDMGQRTSASV
SIFEVVKELPKDQEQKLFYSINDTELFAIHKRSFLLNLPGEVVTKNNKQQRQERRKKR
QFVRSQIRMLANVLRL
Bs3 Cas12b CT₆₅₁₋₁₁₁₂
SEQ ID NO: 84
ETKKTPDERKKAIHKLMEIVQSYDSWTASQKEVWEKELNLLTNMAAFNDEIWKESL
VELHHRIEPYVGQIVSKWRKGLSEGRKNLAGISMWNIDELEDTRRLLISWSKRSRTPG
EANRIETDEPFGSSLLQHIQNVKDDRLKQMANLIIMTALGFKYDKEEKDRYKRWKET
YPACQIILFENLNRYLFNLDRSRRENSRLMKWAHRSIPRTVSMQGEMFGLQVGDVRS
EYSSRFHAKTGAPGIRCHALTEEDLKAGSNTLKRLIEDGFINESELAYLKKGDIIPSQG
GELFVTLSKRYKKDSDNNELTVIHADINAAQNLQKRFWQQNSEVYRVPCQLARMGE
DKLYIPKSQTETIKKYFGKGSFVKNNTEQEVYKWEKSEKMKIKTDTTFDLQDLDGFE
DISKTIELAQEQQKKYLTMFRDPSGYFFNNETWRPQKEYWSIVNNIIKSCLKKKILSN
KVEL

In some embodiments, the N-terminal portion of the reference Cas12b protein comprises the WED-I, REC1, WED-II, RuvC-I and BH domains of a TcCas12b protein, and wherein the C-terminal portion of the reference Cas12b protein comprises the REC2, RuvC-II, Nuc-I, RuvC-III and Nuc-II domains of the TcCas12b protein. In some embodiments, the N-terminal portion of the reference Cas12b protein comprises amino acid residues 1 to 671 of the reference Cas12b protein, and the C-terminal portion of the reference Cas12b protein comprises amino acid residues 672 to 1112 of the reference Cas12b protein, wherein the amino acid residue numbering is according to SEQ ID NO: 88. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 86, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 87. In some embodiments, the N-terminal portion of the reference Cas12b protein comprises the amino acid sequence of SEQ ID NO: 86, and wherein the C-terminal portion of the reference Cas12b protein comprises the amino acid sequence of SEQ ID NO: 87.

Tc Cas12b NT₁₋₆₇₁
SEQ ID NO: 86
MNIHLKELIRMATKSFILKMKTKNNPQLRLSLWKTHELFNFGVAYYMDL
LSLFRQKDLYMHNDEDPDHPVVLKKEEIQERLWMKVRETQQKNGFHGEV
SKDEVLETLRALYEELVPSAVGKSGEANQISNKYLYPLTDPASQSGKGT
ANSGRKPRWKKLKEAGDPSWKDAYEKWEKERQEDPKLKILAALQSFGLI
PLFRPFTENDHKAVISVKWMPKSKNQSVRKFDKDMFNQAIERFLSWESW
NEKVAEDYEKTVSIYESLQKELKGISTKAFEIMERVEKAYEAHLREITF
SNSTYRIGNRAIRGWTEIVKKWMKLDPSAPQGNYLDVVKDYQRRHPRES
GDFKLFELLSRPENQAAWREYPEFLPLYVKYRHAEQRMKTAKKQATFTL
CDPIRHPLWVRYEERSGTNLNKYRLIMNEKEKVVQFDRLICLNADGHYE
EQEDVTVPLAPSQQFDDQIKFSSEDTGKGKHNFSYYHKGINYELKGTLG
GARIQFDREHLLRRQGVKAGNVGRIFLNVTLNIEPMQPFSRSGNLQTSV
GKALKVYVDGYPKVVNFKPKELTEHIKESEKNTLTLGVESLPTGLRVMS
VDLGQRQAAAISIFEVVSEKPDDNKLFYPVKDTDLFAVHRTSFNIKLPG
EKRTERRMLEQQKRDQAIRDLSRKLKFLKNVLNM
Tc Cas12b CT₆₇₂₋₁₁₄₂
SEQ ID NO: 87
QKLEKTDEREKRVNRWIKDREREEENPVYVQEFEMISKVLYSPHSVWVD
QLKSIHRKLEEQLGKEISKWRQSISQGRQGVYGISLKNIEDIEKTRRLL
FRWSMRPENPGEVKQLQPGERFAIDQQNHLNHLKDDRIKKLANQIVMTA
LGYRYDGKRKKWIAKHPACQLVLFEDLSRYAFYDERSRLENRNLMRWSR
REIPKQVAQIGGLYGLLVGEVGAQYSSRFHAKSGAPGIRCRVVKEHELY
ITEGGQKVRNQKFLDSLVENNIIEPDDARRLEPGDLIRDQGGDKFATLD
ERGELVITHADINAAQNLQKRFWTRTHGLYRIRCESREIKDAVVLVPSD
KDQKEKMENLFGIGYLQPFKQENDVYKWVKGEKIKGKKTSSQSDDKELV
SEILQEASVMADELKGNRKTLFRDPSGYVFPKDRWYTGGRYFGTLEHLL
KRKLAERRLFDGGSSRRGLFNGTDSNTNVE

In some embodiments of the foregoing, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises amino acid residues 1 to 783 of the reference Cas12b protein, and the C-terminal portion of the reference Cas12b protein comprises amino acid residues 784 to 1129 of the reference Cas12b protein, wherein the amino acid residue numbering is according to SEQ ID NO: 33. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 5, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 6. In some embodiments, the N-terminal portion of the reference Cas12b protein comprises the amino acid sequence of SEQ ID NO: 5, and wherein the C-terminal portion of the reference Cas12b protein comprises the amino acid sequence of SEQ ID NO: 6.

AaCas12b NT3₁₋₇₈₃
SEQ ID NO: 5
MAVKSMKVKLRLDNMPEIRAGLWKLHTEVNAGVRYYTEWLSLLRQENLY
RRSPNGDGEQECYKTAEECKAELLERLRARQVENGHCGPAGSDDELLQL
ARQLYELLVPQAIGAKGDAQQIARKFLSPLADKDAVGGLGIAKAGNKPR
WVRMREAGEPGWEEEKAKAEARKSTDRTADVLRALADFGLKPLMRVYTD
SDMSSVQWKPLRKGQAVRTWDRDMFQQAIERMMSWESWNQRVGEAYAKL
VEQKSRFEQKNFVGQEHLVQLVNQLQQDMKEASHGLESKEQTAHYLTGR
ALRGSDKVFEKWEKLDPDAPFDLYDTEIKNVQRRNTRRFGSHDLFAKLA
EPKYQALWREDASFLTRYAVYNSIVRKLNHAKMFATFTLPDATAHPIWT
RFDKLGGNLHQYTFLFNEFGEGRHAIRFQKLLTVEDGVAKEVDDVTVPI
SMSAQLDDLLPRDPHELVALYFQDYGAEQHLAGEFGGAKIQYRRDQLNH
LHARRGARDVYLNLSVRVQSQSEARGERRPPYAAVFRLVGDNHRAFVHF
DKLSDYLAEHPDDGKLGSEGLLSGLRVMSVDLGLRTSASISVFRVARKD
ELKPNSEGRVPFCFPIEGNENLVAVHERSQLLKLPGETESKDLRAIREE
RQRTLRQLRTQLAYLRLLVRCGSEDVGRRERSWAKLIEQPMDANQMTPD
WREAFEDELQKLKSLYGICGDREWTEAVYESVRRVWRHMGKQVRDWRKD
VRSGERPKIRGYQKDVVGGNSIEQIEYLERQYKFLKSWSFFGKVSGQV
AaCas12b CT3₇₈₄₋₁₁₂₉
SEQ ID NO: 6
IRAEKGSRFAITLREHIDHAKEDRLKKLADRIIMEALGYVYALDDERGK
GKWVAKYPPCQLILLEELSEYQFNNDRPPSENNQLMQWSHRGVFQELLN
QAQVHDLLVGTMYAAFSSRFDARTGAPGIRCRRVPARCAREQNPEPFPW
WLNKFVAEHKLDGCPLRADDLIPTGEGEFFVSPFSAEEGDFHQIHADLN
AAQNLQRRLWSDFDISQIRLRCDWGEVDGEPVLIPRTTGKRTADSYGNK
VFYTKTGVTYYERERGKKRRKVFAQEELSEEEAELLVEADEAREKSVVL
MRDPSGIINRGDWTRQKEFWSMVNQRIEGYLVKQIRSRVRLQESACENT
GDI

In some embodiments, the N-terminal portion of the reference Cas12b protein comprises WED-I, REC1, WED-II, RuvC-I and BH domains of the reference Cas12b protein, wherein the C-terminal portion of the reference Cas12b protein comprises RuvC-II, Nuc-I, RuvC-III and Nuc-II domains of the reference Cas12b protein, and wherein REC2 domain of the reference Cas12b protein is split between the N-terminal portion of the reference Cas12b protein and the C-terminal portion of the reference Cas12b protein.

In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein. In some embodiments, the N-terminal portion of the reference Cas12b protein comprises amino acid residues 1 to 518 of the reference Cas12b protein, and the C-terminal portion of the reference Cas12b protein comprises amino acid residues 519 to 1129 of the reference Cas12b protein, wherein the amino acid residue numbering is according to SEQ ID NO: 33. In some embodiments, the N-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 1, and wherein the C-terminal portion of the reference Cas12b protein comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 2. In some embodiments, the N-terminal portion of the reference Cas12b protein comprises the amino acid sequence of SEQ ID NO: 1, and wherein the C-terminal portion of the reference Cas12b protein comprises the amino acid sequence of SEQ ID NO: 2.

AaCas12b NT1₁₋₅₁₈
SEQ ID NO: 1
MAVKSMKVKLRLDNMPEIRAGLWKLHTEVNAGVRYYTEWLSLLRQENLY
RRSPNGDGEQECYKTAEECKAELLERLRARQVENGHCGPAGSDDELLQL
ARQLYELLVPQAIGAKGDAQQIARKFLSPLADKDAVGGLGIAKAGNKPR
WVRMREAGEPGWEEEKAKAEARKSTDRTADVLRALADFGLKPLMRVYTD
SDMSSVQWKPLRKGQAVRTWDRDMFQQAIERMMSWESWNQRVGEAYAKL
VEQKSRFEQKNFVGQEHLVQLVNQLQQDMKEASHGLESKEQTAHYLTGR
ALRGSDKVFEKWEKLDPDAPFDLYDTEIKNVQRRNTRRFGSHDLFAKLA
EPKYQALWREDASFLTRYAVYNSIVRKLNHAKMFATFTLPDATAHPIWT
RFDKLGGNLHQYTFLFNEFGEGRHAIRFQKLLTVEDGVAKEVDDVTVPI
SMSAQLDDLLPRDPHELVALYFQDYGAEQHLAGEFGGAKIQYRRDQLNH
LHARRGARDVYLNLSVRVQSQSEARGER
AaCas12b CT1₅₁₉₋₁₁₂₉
SEQ ID NO: 2
RPPYAAVFRLVGDNHRAFVHFDKLSDYLAEHPDDGKLGSEGLLSGLRVM
SVDLGLRTSASISVFRVARKDELKPNSEGRVPFCFPIEGNENLVAVHER
SQLLKLPGETESKDLRAIREERQRTLRQLRTQLAYLRLLVRCGSEDVGR
RERSWAKLIEQPMDANQMTPDWREAFEDELQKLKSLYGICGDREWTEAV
YESVRRVWRHMGKQVRDWRKDVRSGERPKIRGYQKDVVGGNSIEQIEYL
ERQYKFLKSWSFFGKVSGQVIRAEKGSRFAITLREHIDHAKEDRLKKLA
DRIIMEALGYVYALDDERGKGKWVAKYPPCQLILLEELSEYQFNNDRPP
SENNQLMQWSHRGVFQELLNQAQVHDLLVGTMYAAFSSRFDARTGAPGI
RCRRVPARCAREQNPEPFPWWLNKFVAEHKLDGCPLRADDLIPTGEGEF
FVSPFSAEEGDFHQIHADLNAAQNLQRRLWSDFDISQIRLRCDWGEVDG
EPVLIPRTTGKRTADSYGNKVFYTKTGVTYYERERGKKRRKVFAQEELS
EEEAELLVEADEAREKSVVLMRDPSGIINRGDWTRQKEFWSMVNQRIEG
YLVKQIRSRVRLQESACENTGDI

The split point is typically designed in silico and cloned into the constructs. Together, the two split Cas12b portions, the N-terminal and C-terminal parts, form a functional Cas12b protein, comprising preferably at least 70% or more of the wildtype amino acid sequence, such as any one of at least 75%, 80%, 85%, 90%, 95%, 98%, 99% or more of the wildtype amino acid sequence. Some trimming and mutants are envisaged. Non-functional domains may be removed entirely. For all split Cas12b systems, the two split Cas12b portions may be brought together and that the desired Cas12b function is restored or reconstituted.

Activities of the reconstituted Cas12b protein or CRISPR complex (Cas12b+guide RNA complex) can be assessed using known methods in the art. For example, nuclease activity within a cell can be assessed using a T7 endonuclease I (T7EI) assay as described in Example 1. Gene-editing activity can also be assessed by DNA sequencing.

In some embodiments, the reference Cas12b protein is split into more than two portions. In some embodiments, the reference Cas12b protein may be split into three portions. In some embodiments, the reference Cas12b protein may be split into four portions. In some embodiments, the reference Cas12b protein may be split into five portions. In some embodiments, the reference Cas12b protein may be split into six portions.

### Dimerization Domains

The split Cas12b polypeptides may each comprise one or more dimerization domains. In some embodiments, the first polypeptide comprises a first dimerization domain fused to the first split Cas12b portion, and the second polypeptide comprises a second dimerization domain fused to the second split Cas12b portion. The dimerization domain may be fused to the split Cas12b portion via a peptide linker (e.g., a flexible peptide linker such as a GS linker) or a chemical bond. In some embodiments, the dimerization domain is fused to the N-terminus of the split Cas12b portion. In some embodiments, the dimerization domain is fused to the C-terminus of the split Cas12b portion.

In some embodiments, the split Cas12b polypeptides do not comprise any dimerization domains.

In some embodiments, the dimerization domains promotes association of the two split Cas12b portions. In some embodiments, the split Cas12b portions are induced to associate or dimerize into a functional Cas12b protein by an inducer. In some embodiments, the split Cas12b polypeptides comprise inducible dimerization domains. In some embodiments, the dimerization domains are not inducible dimerization domains, i.e., the dimerization domains dimerize without the presence of an inducer.

An inducer may be an inducing energy source or an inducing molecule other than a guide RNA (e.g., a sgRNA). The inducer acts to reconstitute two split Cas12b portions into a functional Cas12b protein via induced dimerization of the dimerization domains. In some embodiments, the inducer brings the two split Cas12b portions together through the action of induced association of the inducible dimerization domains. In some embodiments, without the inducer, the two split Cas12b portions do not associate with each other to reconstitute into a functional Cas12b protein. In some embodiments, without the inducer, the two split Cas12b portions may associate with each other to reconstitute into a functional Cas12b protein in the presence of a guide RNA (e.g., a sgRNA).

The inducer of the present application may be heat, ultrasound, electromagnetic energy or a chemical compound. In some embodiments, the inducer is an antibiotic, a small molecule, a hormone, a hormone derivative, a steroid or a steroid derivative. In some embodiments, the inducer is abscisic acid (ABA), doxycycline (DOX), cumate, rapamycin, 4-hydroxytamoxifen (4OHT), estrogen or ecdysone. In some embodiments, the split Cas12b system is an inducer-controlled system selected from the group consisting of antibiotic based inducible systems, electromagnetic energy based inducible systems, small molecule based inducible systems, nuclear receptor based inducible systems and hormone based inducible systems. In some embodiments, the split Cas12b system is an inducer-controlled system is selected from the group consisting of tetracycline (Tet)/DOX inducible systems, light inducible systems, ABA inducible systems, cumate repressor/operator systems, 4OHT/estrogen inducible systems, ecdysone-based inducible systems and FKBP12/FRAP (FKBP12-rapamycin complex) inducible systems. Such inducers are also discussed herein and in PCT/US2013/051418, which is incorporated herein by reference in its entirety. FRB/FKBP/Rapamycin systems have been described in Paulmurugan and Gambhir, Cancer Res, Aug. 15, 2005 65; 7413; and Crabtree et al., Chemistry & Biology 13, 99-107, January 2006, which are incorporated herein by reference in their entirety.

In some embodiments, wherein the first polypeptide comprises a first dimerization domain and the second polypeptide comprises a second dimerization domain, the first dimerization domain and the second dimerization domain associate with each other in the presence of an inducer. In some embodiments, the first dimerization domain is FK506 binding protein (FKBP) and the second dimerization domain is FKBP-rapamycin-binding domain (FRB). In some embodiments, the first dimerization domain is FRB and the second dimerization domain is FKBP. In some embodiments, the inducer is rapamycin. In some embodiments, the FKBP domain comprises the amino acid sequence of SEQ ID NO: 31. In some embodiments, the FRB domain comprises the amino acid sequence of SEQ ID NO: 32.

FKBP domain
SEQ ID NO: 31
MGVQVETISPGDGRTFPKRGQTCVVHYTGMLEDGKKFDSSRDRNKPFKF
MLGKQEVIRGWEEGVAQMSVGQRAKLTISPDYAYGATGHPGIIPPHATL
VFDVELLKLE
FRB domain
SEQ ID NO: 32
EMWHEGLEEASRLYFGERNVKGMFEVLEPLHAMMERGPQTLKETSFNQA
YGRDLMEAQEWCRKYMKSGNVKDLTQAWDLYYHVFRRISKQ

In some embodiments, the first polypeptide comprises from the N-terminus to the C-terminus: an FKBP domain, an optional peptide linker and a first split Cas12b portion; and the second polypeptide comprising from the N-terminus to the C-terminus: an FRB domain, an optional peptide linker and a second split Cas12b portion. In some embodiments, the first polypeptide comprises from the N-terminus to the C-terminus: an FKBP domain, an optional peptide linker and a first split Cas12b portion; and the second polypeptide comprising from the N-terminus to the C-terminus: a second split Cas12b portion, an optional peptide linker, and a second split Cas12b portion. In some embodiments, the first polypeptide comprises from the N-terminus to the C-terminus: a first split Cas12b portion, an optional peptide linker and an FKBP domain; and the second polypeptide comprising from the N-terminus to the C-terminus: an FRB domain, an optional peptide linker and a second split Cas12b portion. In some embodiments, the first polypeptide comprises from the N-terminus to the C-terminus: a first split Cas12b portion, an optional peptide linker and an FKBP domain; and the second polypeptide comprising from the N-terminus to the C-terminus: a second split Cas12b portion, an optional peptide linker, and an FRB domain. Constructs have the first split Cas12b portion swapped with the second split Cas12b portion with respect to the above constructs are also contemplated.

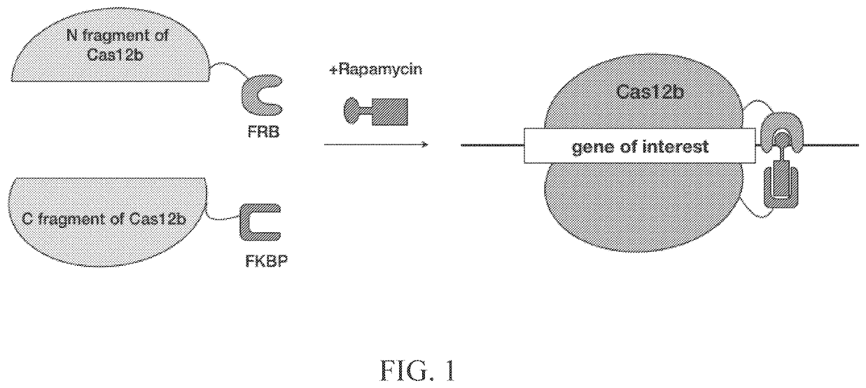

An exemplary inducer-controlled split Cas12b system comprising a first polypeptide having an N-terminus split Cas12b portion fused to an FRB domain, and a second polypeptide having a C-terminus split Cas12b portion fused to an FKBP domain, and rapamycin as the inducer, is shown in FIG. 1. In some embodiments, the first polypeptide comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 11, and wherein the second polypeptide comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 12. In some embodiments, the first polypeptide comprises the amino acid sequence of SEQ ID NO: 11, and wherein the second polypeptide comprises the amino acid sequence of SEQ ID NO: 12. In some embodiments, the first polypeptide comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 13, and wherein the second polypeptide comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 14. In some embodiments, the first polypeptide comprises the amino acid sequence of SEQ ID NO: 13, and wherein the second polypeptide comprises the amino acid sequence of SEQ ID NO: 14. In some embodiments, the first polypeptide comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 15, and wherein the second polypeptide comprises an amino acid sequence having at least about 85% (e.g., at least about any one of 90%, 91%, 92%, 93%, 94%, 95%, 96%, 97%, 98%, or 99%) sequence identity to the amino acid sequence of SEQ ID NO: 16. In some embodiments, the first polypeptide comprises the amino acid sequence of SEQ ID NO: 15, and wherein the second polypeptide comprises the amino acid sequence of SEQ ID NO: 16.

In some embodiments, the engineered CRISPR-Cas system is induced by rapamycin at a sufficient amount and for a suitable duration. In some embodiments, rapamycin induction may last various days. In some embodiments, the rapamycin induction lasts about 1, 2, 3, 4, 5, 6, 7, 8, 9, 10, 11, 12, 13, 14, or 15 days. The suitable amount of rapamycin for inducing the engineered CRISPR-Cas system may be at least about any one of 5 nM, 25 nM, 50 nM, 75 nM, 100 nM, 125 nM, 150 nM, 175 nM, 200 nM, 250 nM, 300 nM, 400 nM, or 500 nM. In some embodiments, the suitable amount of rapamycin for inducing the engineered CRISPR-Cas system is no more than about any one of 500 nM, 400 nM, 300 nM, 250 nM, 200 nM, 175 nM, 150 nM, 125 nM, 100 nM, 75 nM, 50 nM, 25 nM, 5 nM or less. In some embodiments, the rapamycin is applied at about 100 nM and for about 60 hours, e.g., for human embryonic kidney 293T (HEK293T) cell lines. The in vitro rapamycin induction amount and duration may be extrapolated for therapeutic use in vivo. However, it is also envisaged that the standard dosage for administering rapamycin to a subject is used here as well. By the “standard dosage”, it is meant the dosage under rapamycin's normal therapeutic use or primary indication (i.e., the dose used when rapamycin is administered for use to prevent organ rejection).

In some embodiments, the pair of split Cas12b polypeptides are separate and inactive until induced dimerization of the dimerization domains (e.g., FRB and FKBP), which results in reassembly of a functional Cas12b nuclease. In some embodiments, the first split Cas12b polypeptide comprising a first half of an inducible dimer (e.g., FRB) is delivered separately and/or is localized separately from the second split Cas12b polypeptide comprising a second half of an inducible dimer (e.g., FKBP).

Other exemplary FKBP-based inducible systems that may be used in inducer-controlled split Cas12b systems described herein include, but are not limited to, FKBP which dimerizes with CalcineurinA (CNA), in the presence of FK506; FKBP which dimerizes with CyP-Fas, in the presence of FKCsA; FKBP which dimerizes with FRB, in the presence of Rapamycin; GyrB which dimerizes with GryB, in the presence of Coumermycin; GAI which dimerizes with GID1, in the presence of Gibberellin; or Snap-tag which dimerizes with HaloTag, in the presence of HaXS.

Alternatives within the FKBP family itself are also contemplated. For example, FKBP, which homodimerizes (i.e., one FKBP dimerizes with another FKBP) in the presence of FK1012.

In some embodiments, the dimerization domain is FKBP and the inducer is FK1012. In some embodiments, the dimerization domain is GryB and the inducer is coumermycin. In some embodiments, the dimerization domain is ABA and the inducer is Gibberellin.

In some embodiments, the split Cas12b portions may be auto-induced (i.e., auto-activated or self-induced) to associate/dimerize into a functional Cas12b protein without the presence of an inducer. Without being bound by any theory or hypothesis, auto-induction of the split Cas12b portions may be mediated by binding to a guide RNA, such as sgRNA. In some embodiments, the first polypeptide and the second polypeptide do not comprise dimerization domains. In some embodiments, the first polypeptide and the second polypeptide comprise dimerization domains.

In some embodiments, the reconstituted Cas12b protein of the split Cas12b systems described herein (including inducer-controlled and auto-inducible systems) has an editing efficiency of at least 70% (such as at least about any of 70%, 75%, 80%, 85%, 90%, 95%, 98%, 99% or more efficiency, or 100% efficiency) of the editing efficiency of the reference Cas12b protein.

In some embodiments, the reconstituted Cas12b protein of an inducer-controlled split Cas12b systems described herein has an editing efficiency of no more than 50% (such as no more than about any of 50%, 45%, 40%, 35%, 30%, 25%, 20%, 15%, 10%, 5%, or less efficiency, or 0% efficiency) without the presence of an inducer (i.e., due to auto-induction) of the editing efficiency of the reference Cas12b protein.

### Additional Protein Domains and Components

In addition to the split Cas12b portions and optionally dimerization domains, the split Cas12b polypeptides may comprise additional protein domains and/or components, such as linkers, nuclear localization/exportation sequences, and/or reporter proteins.

In some embodiments, the present application provides a split Cas12b system having one or more (e.g., 1, 2, 3, 4, 5, 6, or more) functional domains associated with (i.e., bound to or fused to) one or both split Cas12b portions. The functional domain(s) may be provided as part of the first and/or second split Cas12b polypeptides, as fusions within that construct. The functional domains are typically fused to other parts in the split Cas12b polypeptides (e.g., split Cas12b portions) via a peptide linker, such as GS linker. An exemplary linker sequence is SRGGSGSSGGSGGSGGSG (SEQ ID NO: 71). In some embodiments, the functional domains may have one or more activities selected from methylase activity, demethylase activity, transcription activation activity, transcription repression activity, transcription release factor activity, histone modification activity, RNA cleavage activity, DNA cleavage activity, DNA integration activity or nucleic acid binding activity. In some embodiments, the one or more functional domains are transcriptional activation domains (i.e., transactivation domains) or repressor domains. In some embodiments, the one or more functional domains are histone-modifying domains. In some embodiments, the one or more functional domains are transposase domains, HR (Homologous Recombination) machinery domains, recombinase domains, and/or integrase domains. Although different domains may be combined with each other within a single split Cas12b system, it is preferred that all the functional domains are either activators or repressors. In some embodiments, the reference Cas12b protein is enzymatically inactive. The functional domains can be used to repurpose the function of the split Cas12b system based on a catalytically dead Cas12b.

In some embodiments, the first polypeptide further comprises a functional domain fused to the N-terminal portion of the reference Cas12b protein, and/or the second polypeptide further comprises a functional domain fused to the C-terminal portion of the reference Cas12b protein. In some embodiments, the first polypeptide comprises a first function domain, and the second polypeptide comprises a second functional domain. In some embodiments, only one or the first polypeptide and the second polypeptide comprises one or more functional domains. In some embodiments, the functional domain is selected from the group consisting of a translation initiator domain, a transcription repressor domain, a transactivation domain, an epigenetic modification domain, and a nuclease domain.

In some embodiments, the first polypeptide comprises a transactivation domain fused to the N-terminal portion of the reference Cas12b protein, and/or the second polypeptide comprises a transactivation domain fused to the C-terminal portion of the reference Cas12b protein. In some embodiments, the first polypeptide comprises a first transactivation domain, and the second polypeptide comprises a second transactivation domain. In some embodiments, the first transactivation domain is the same as the second transactivation domain. In some embodiments, the first transactivation domain and the second transactivation domain are different. In some embodiments, the transactivation domain is fused to the N-terminus of a split Cas12b portion. In some embodiments, the transactivation domain is fused to the C-terminus of a split Cas12b portion. In some embodiments, the transactivation domain is selected from the group consisting of VP64, p65, HSF1, VP16, MyoD1, HSF1, RTA, SETT/9, and combinations thereof. In some embodiments, the transactivation domain comprises VP64, p65 and HSF1.

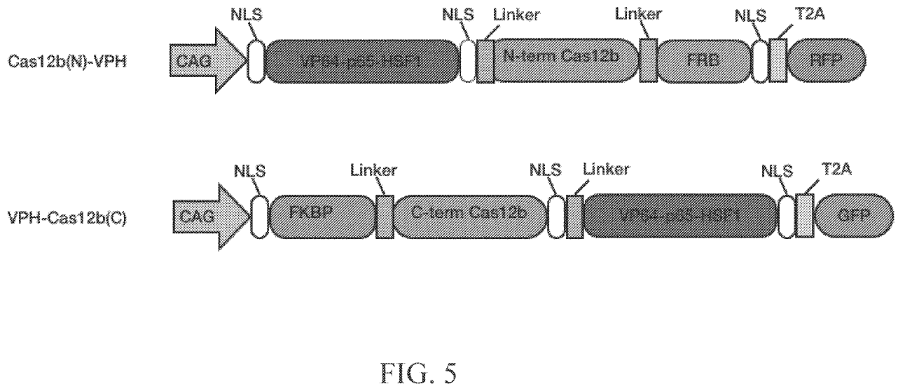

For example, FIG. 5 shows an exemplary inducer-controlled split Cas12b system useful for sequence-specific transcriptional activation of a target nucleic acid (e.g., target DNA). This exemplary system comprises a first polypeptide comprising from the N-terminus to the C-terminus: transactivation domains comprising VP64, p65 and HSF1, a first optional linker, and a first split Cas12b portion (e.g., an N-terminal Cas12b portion), a second optional linker, and a FRB domain; and a second polypeptide comprising from the N-terminus to the C-terminus: a FKBP domain, a first optional linker, a second split Cas12b portion (e.g., a C-terminal Cas12b portion), a second optional linker, and transactivation domains comprising VP64, p65 and HSF1. The first split Cas12b portion and the second Cas12b portion reconstitute into an enzymatically inactive Cas12b protein (dCas12b) upon binding to the guide RNA, forming a CRISPR complex. In some embodiments, reconstitution of the dCas12b requires an inducer, such as rapamycin, that promotes dimerization of the FRB and FKBP domains. Sequence-specific transcriptional activation is induced by the VP64, p65 and HSF1 domains upon binding of the CRISPR complex to a target DNA. Variations of this exemplary split Cas12b system are contemplated, in which the FRB and FKBP domains may be omitted for auto-inducing systems, and the relative order of the split Cas12b portion, the transactivation domains and the optional dimerization domain within each polypeptide may differ from those shown in FIG. 5.

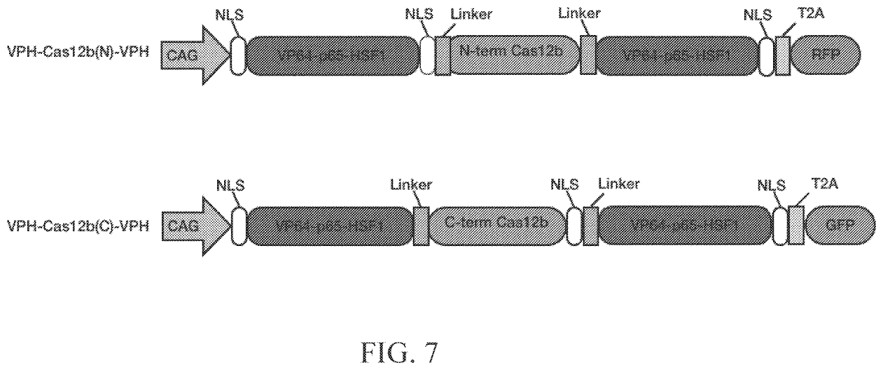

FIG. 7 shows an exemplary auto-inducing split Cas12b system useful for sequence-specific transcriptional activation of a target nucleic acid (e.g., target DNA). This exemplary system comprises a first polypeptide comprising from the N-terminus to the C-terminus: transactivation domains comprising VP64, p65 and HSF1, a first optional linker, and a first split Cas12b portion (e.g., an N-terminal Cas12b portion), a second optional linker, and transactivation domains comprising VP64, p65 and HSF1; and a second polypeptide comprising from the N-terminus to the C-terminus: transactivation domains comprising VP64, p65 and HSF1, a first optional linker, a second split Cas12b portion (e.g., a C-terminal Cas12b portion), a second optional linker, and transactivation domains comprising VP64, p65 and HSF1. Reconstitution of the CRISPR complex does not require an inducer.

In some embodiments, the first polypeptide comprises a transcription repressor domain fused to the N-terminal portion of the reference Cas12b protein, and/or the second polypeptide comprises a transcription repressor domain fused to the C-terminal portion of the reference Cas12b protein. In some embodiments, the first polypeptide comprises a first transcription repressor domain, and the second polypeptide comprises a second transcription repressor domain. In some embodiments, the first transcription repressor domain is the same as the second transcription repressor domain. In some embodiments, the first transcription repressor domain and the second transcription repressor domain are different. In some embodiments, the transcription repressor domain is fused to the N-terminus of a split Cas12b portion. In some embodiments, the transcription repressor domain is fused to the C-terminus of a split Cas12b portion. In some embodiments, the functional domain is a transcription repressor domain. In some embodiments, the transcription repressor is selected from the group consisting of Krüppel associated box (KRAB), EnR, NuE, NcoR, SID, and SID4X.

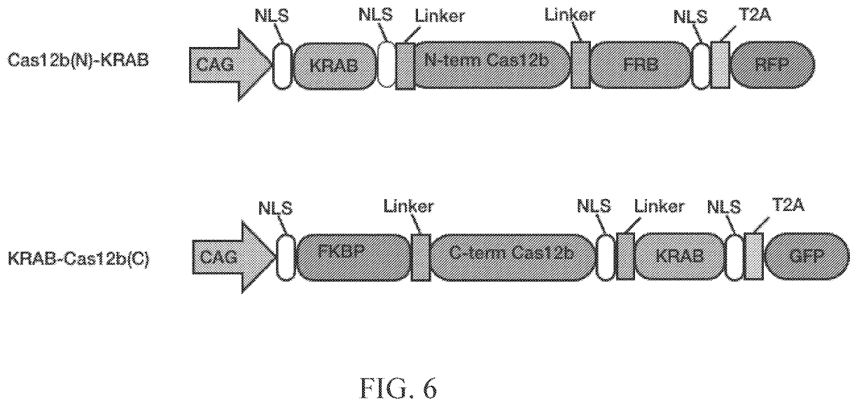

For example, FIG. 6 shows an exemplary split Cas12b system useful for sequence-specific transcriptional repression of a target nucleic acid (e.g., target DNA). This exemplary system comprises a first polypeptide comprising from the N-terminus to the C-terminus: a KRAB domain, a first optional linker, and a first split Cas12b portion (e.g., an N-terminal Cas12b portion), a second optional linker, and a FRB domain; and a second polypeptide comprising from the N-terminus to the C-terminus: a FKBP domain, a first optional linker, a second split Cas12b portion (e.g., a C-terminal Cas12b portion), a second optional linker, and KRAB domain. The first split Cas12b portion and the second Cas12b portion reconstitute into an enzymatically inactive Cas12b protein (dCas12b) upon binding to the guide RNA, forming a CRISPR complex. In some embodiments, reconstitution of the dCas12b requires an inducer, such as rapamycin, that promotes dimerization of the FRB and FKBP domains. Sequence-specific transcriptional repression is induced by the KRAB domain upon binding of the CRISPR complex to a target DNA. Variations of this exemplary split Cas12b system are contemplated, in which the FRB and FKBP domains may be omitted for auto-inducing systems, and the relative order of the split Cas12b portion, the transcription repressor domain and the optional dimerization domain within each polypeptide may differ from those shown in FIG. 6.

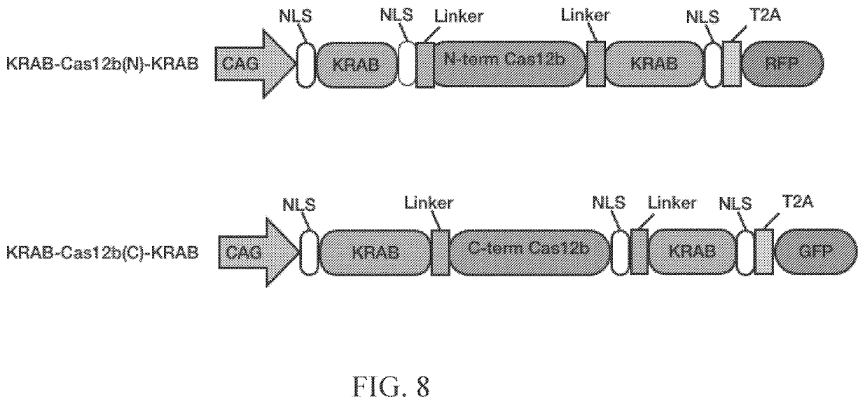

FIG. 8 shows an exemplary auto-inducing split Cas12b system useful for sequence-specific transcriptional activation of a target nucleic acid (e.g., target DNA). This exemplary system comprises a first polypeptide comprising from the N-terminus to the C-terminus: KRAB domain, a first optional linker, and a first split Cas12b portion (e.g., an N-terminal Cas12b portion), a second optional linker, and KRAB domain; and a second polypeptide comprising from the N-terminus to the C-terminus: KRAB domain, a first optional linker, a second split Cas12b portion (e.g., a C-terminal Cas12b portion), a second optional linker, and KRAB domain. Reconstitution of the CRISPR complex does not require an inducer.

In some embodiments, the split Cas12b polypeptides comprise one or more nuclear localization sequences (NLSs) and/or one or more nuclear exportation sequences (NESs).

In some embodiments, the first polypeptide and/or the second polypeptide further comprises a nuclear localization signal (NLS). Exemplary NLS sequences include, for example, PKKKRKVPG (SEQ ID NO: 34) and ASPKKKRKV (SEQ ID NO: 35). In some embodiments, one or more (e.g., two or three) NLSs may be used in operable linkage to the first split Cas12b portion and optionally the first dimerization domain. In some embodiments, one or more (e.g., two or three) NLSs may be used in operable linkage to the second split Cas12b portion and optionally the second dimerization domain. In some embodiments, one or more (e.g., two or three) NESs may be used in operable linkage to the first split Cas12b portion and optionally the first dimerization domain. In some embodiments, one or more (e.g., two or three) NESs may be used in operable linkage to the second split Cas12b portion and optionally the second dimerization domain. The NLSs and/or the NESs preferably flank the split Cas12b portions, i.e., one NLS may be positioned at the N-terminus of the first split Cas12b portion and one NLS may be at the C-terminus of the first split Cas12b portion. Similarly, one NES may be positioned at the N-terminus of the second split Cas12b portion and one NES may be at the C-terminus of the second split Cas12b portion.

In some embodiments, the NES functions to localize the second Cas12b fusion construct outside of the nucleus, at least until the inducer is provided (e.g., at least until an energy source is provided to the inducer to perform its function). The presence of the inducer stimulates dimerization of the two split Cas12b polypeptides within the cytoplasm and makes it thermodynamically worthwhile for the dimerized, first and second split Cas12b polypeptides to localize to the nucleus. A skilled artisan may use the NES and/or NLS to shift an equilibrium (the equilibrium of nuclear transport) to a desired direction.

In some embodiments, the first split Cas12b polypeptide and/or the second split Cas12b polypeptide comprises a reporter protein, such as a fluorescent protein, e.g., GFP. Such system could allow imaging of genomic loci (see, for example, “Dynamic Imaging of Genomic Loci in Living Human Cells by an Optimized CRISPR/Cas System” Chen B et al. Cell 2013). In some embodiments, the split Cas12b system is an inducible system that can be used to image genomic loci.

### Guide RNA

The split Cas12b systems of the present application may comprise any suitable guide RNAs. A guide RNA (gRNA) may comprise a guide sequence capable of hybridizing to a target sequence in a target nucleic acid of interest, such as a genomic locus of interest in a cell. In some embodiments, the guide RNA comprises a CRISPR RNA (crRNA) molecule and a trans-activating CRISPR RNA (tracrRNA) molecule. In some embodiments, the split Cas12b system is a dual RNA system, comprising a crRNA and a tracrRNA. In some embodiments, the guide RNA is a single-guide RNA (sgRNA). In some embodiments, the sgRNA comprises a crRNA sequence comprising the guide sequence. In some embodiments, the sgRNA comprises a tracrRNA sequence. In some embodiments, the sgRNA comprises a tracrRNA sequence fused to a crRNA sequence.

In some embodiments, the gRNA is a multiplex gRNA that target more than one target nucleic acids. In some embodiments, the gRNA comprises a plurality (e.g., 2, 3, 4, 5, 6, or more) crRNA sequences, wherein each crRNA sequences comprises a different target sequence.

The guide sequence may have a suitable length. In some embodiments, the guide sequence is between about 18 to about 35 nucleotides, including, for example, any one of 18, 19, 20, 21, 22, 23, 24, 25, 26, 27, 28, 29, 30, 31, 32, 33, 34 or 35 nucleotides. The guide sequence may have at least about 70%, 75%, 80%, 85%, 90%, 95%, 98%, 99%, or 100% complementarity to a target sequence of the target nucleic acid.

In some embodiments, the gRNA comprises a cognate crRNA sequence and/or tracrRNA sequence that corresponds to the reference Cas12b protein. In some embodiments, the gRNA comprises a non-cognate crRNA sequence and/or tracrRNA sequence that are not naturally found in the CRISPR loci of the reference Cas12b protein. For example, cognate tracrRNA and crRNA sequences of AaCas12b, AkCas12b, AmCas12b, BhCas12b, BsCas12b, Bs3Cas12b, LsCas12b and SbCas12b, as well as exemplary sgRNA sequences have been described in FIG. S4 and FIG. S8 of Teng F. et al., Cell Discovery (2019) 5:23, which are incorporated herein by reference in their entirety.

In some embodiments, the tracrRNA comprises the nucleic acid sequence as follows:5′ GUCUAAAGGACAGAAUUUUUCAACGGGUGUGCCAAUGGCCACUUUCC AGGUGGCAAAGCCCGUUGAACUUCUCAAAAAGAACGCUCGCUCAGUGUUCUGA C-3′ (SEQ ID NO: 36). In some embodiments, the crRNA comprises the nucleic acid sequence as follows: 5′-GUCGGAUCACUGAGCGAGCGAUCUGAGAAGUGGCAC-Nₓ-3′ (SEQ ID NO: 37), wherein Nₓ represents a nucleic acid sequence having x consecutive nucleotides, wherein each N is independently chosen from A, G, C or U, and wherein X is an integer between 18 and 35. In some embodiments, X is 20. In some embodiments, Nₓ comprises the guide sequence.

In some embodiments, the sgRNA comprises a nucleic acid sequence selected from the following:

(SEQ ID NO: 38)
5′-GUCUAAAGGACAGAAUUUUUCAACGGGUGUGCCAAUGGCCACUUUCCAGGU
GGCAAAGCCCGUUGAACUUCUCAAAAAGAACGCUCGCUCAGUGUUCUGACGUC
GG AUCACUGAGCGAGCGAUCUGAGAAGUGGCAC-Nₓ-3′;
(SEQ ID NO: 39)
5′-AACUGUCUAAAGGACAGAAUUUUUCAACGGGUGUGCCAAUGGCCACUUUCC
AGGUGGCAAAGCCCGUUGAACUUCUCAAAAAGAACGCUCGCUCAGUGUUCUGA
CG UCGGAUCACUGAGCGAGCGAUCUGAGAAGUGGCAC-Nₓ-3′;
(SEQ ID NO: 40)
5′-CUGUCUAAAGGACAGAAUUUUUCAACGGGUGUGCCAAUGGCCACUUUCCAG
GUGGCAAAGCCCGUUGAACUUCUCAAAAAGAACGCUCGCUCAGUGUUCUGACG
UC GGAUCACUGAGCGAGCGAUCUGAGAAGUGGCAC-Nₓ-3′;
(SEQ ID NO: 41)
5′-GUCUAAAGGACAGAAUUUUUCAACGGGUGUGCCAAUGGCCACUUUCCAGGU
GGCAAAGCCCGUUGAACUUCUCAAAAAGAACGCUCGCUCAGUGUUAUCACUGA
GC GAGCGAUCUGAGAAGUGGCAC-Nₓ-3′;
(SEQ ID NO: 42)
5′-GUCUAAAGGACAGAAUUUUUCAACGGGUGUGCCAAUGGCCACUUUCCAGGU
GGCAAAGCCCGUUGAACUUCUCAAAAAGAACGAUCUGAGAAGUGGCAC-Nₓ-3′;
(SEQ ID NO: 43)
5′-GUCUAAAGGACAGAAUUUUUCAACGGGUGUGCCAAUGGCCACUUUCCAGGU
GGCAAAGCCCGUUGAACUUCUCAAAAAGCUGAGAAGUGGCAC-Nₓ-3′;
(SEQ ID NO: 44)
5′-GUCUAAAGGACAGAAUUUUUCAACGGGUGUGCCAAUGGCCACUUUCCAGGU
GGCAAAGCCCGUUGAACUUCUCAAAGCUGAGAAGUGGCAC-Nₓ-3′;
(SEQ ID NO: 45)
5′-GUCUAAAGGACAGAAUUUUUCAACGGGUGUGCCAAUGGCCACUUUCCAGGU
GGCAAAGCCCGUUGAACUUCUCAAAACUGAGAAGUGGCAC-Nₓ-3′;
(SEQ ID NO: 46)
5′-GUCUAAAGGACAGAAUUUUUCAACGGGUGUGCCAAUGGCCACUUUCCAGGU
GGCAAAGCCCGUUGAACUUCUCAAGCGAGAAGUGGCAC-Nₓ-3′;
(SEQ ID NO: 47)
5′-GUCUAAAGGACAGAAUUUUUCAACGGGUGUGCCAAUGGCCACUUUCCAGGU
GGCAAAGCCCGUUGAACUUCUAAGCAGAAGUGGCAC-Nₓ-3′;
and
(SEQ ID NO: 48)
5′-GUCUAAAGGACAGAAUUUUUCAACGGGUGUGCCAAUGGCCACUUUCCAGGU
GGCAAAGCCCGUUGAACUUCAAGCGAAGUGGCAC-Nₓ-3′;

wherein Nₓ represents a nucleic acid sequence having x consecutive nucleotides, wherein each N is independently chosen from A, G, C or U, and wherein X is an integer between 18 and 35. In some embodiments, X is 20. In some embodiments, Nₓ comprises the guide sequence. Other exemplary sgRNA scaffolds for Cas12b proteins have been disclosed, for example, in WO2019/127087, which is incorporated herein by reference in its entirety.

In some embodiments, the sgRNA comprises from the 5′ to the 3′: a first stem loop, a second stem loop, a third stem loop and a fourth stem loop. In some embodiments, the sgRNA comprises artificial sgRNA scaffold 13 (artsgRNA13). In some embodiments, the sgRNA comprises a nucleic acid sequence as follows:

(SEQ ID NO: 7)
5′GUCGUCUAUAGGACGGCGAGUUUUUCAACGGGUGUGCCAAUGGCCACU
UUCCAGGUGGCAAAGCCCGUUGAGCUUCAAGAAGUGGCAC-Nₓ-3′
or
(SEQ ID NO: 96)
5′GUCGUCUAUAGGACGGCGAGUUUUUCAACGGGUGUGCCAAUGGCCACU
UUCCAGGUGGCAAAGCCCGUUGAGCUUCAAAGAAGUGGCAC-Nₓ-3′;

wherein Nₓ represents a nucleic acid sequence having x consecutive nucleotides, wherein each N is independently chosen from A, G, C or U, and wherein X is an integer between 18 and 35.

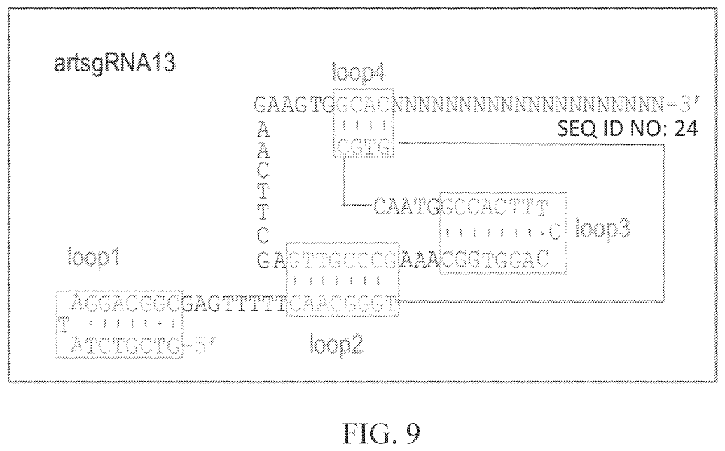

In some embodiments, the guide RNA is a truncated sgRNA comprising a tracrRNA sequence and a crRNA sequence comprising the guide sequence, and wherein compared to a full-length sgRNA comprising a wildtype tracrRNA sequence and a wildtype crRNA sequence corresponding to the reference Cas12b protein, the truncated sgRNAs lacks one or more stem loops. The full-length sgRNA may comprise various number of stem loops. By way of example, as illustrated in FIG. 9, the full-length sgRNA may comprise from the 5′ to the 3′: a first stem loop, a second stem loop, a third stem loop and a fourth stem loop. Without being bound by any theory or hypothesis, it is believed that one or more of the stem loops may mediate auto-induction of the split Cas12b polypeptides.

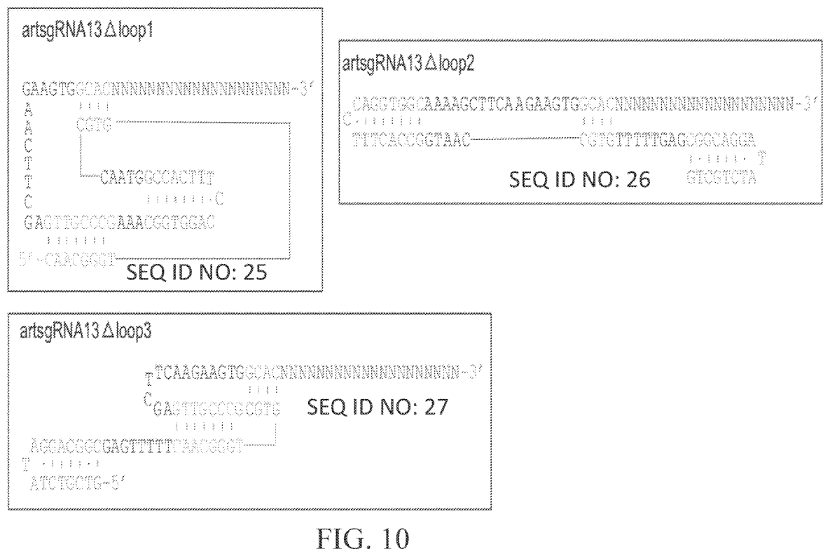

Exemplary truncated sgRNA scaffolds are shown in FIG. 10. In some embodiments, the truncated sgRNA lacks the first stem loop. In some embodiments, the sgRNA comprises artsgRNA13Δloop1. In some embodiments, the sgRNA comprises a nucleic acid sequence as follows:

(SEQ ID NO: 8)
5′CAACGGGUGUGCCAAUGGCCACUUUCCAGGUGGCAAAGCCCGUUGAGC
UUCAAGAAGUGGCAC-Nₓ-3′
or
(SEQ ID NO: 97)
5′CAACGGGUGUGCCAAUGGCCACUUUCCAGGUGGCAAAGCCCGUUGAGC
UUCAAAGAAGUGGCAC-Nₓ-3′;

wherein Nₓ represents a nucleic acid sequence having x consecutive nucleotides, wherein each N is independently chosen from A, G, C or U, and wherein X is an integer between 18 and 35. In some embodiments, X is 20.

In some embodiments, the truncated sgRNA lacks the second stem loop. In some embodiments, the sgRNA comprises artsgRNA13Δloop2. In some embodiments, the sgRNA comprises a nucleic acid sequence as follows:

(SEQ ID NO: 9)
5′GUCGUCUAUAGGACGGCGAGUUUUUGUGCCAAUGGCCACUUUCCAGGU
GGCAAAAGCUUCAAGAAGUGGCAC-Nₓ-3′
or
(SEQ ID NO: 98)
5′GUCGUCUAUAGGACGGCGAGUUUUUGUGCCAAUGGCCACUUUCCAGGU
GGCAAAAGCUUCAAAGAAGUGGCAC-Nₓ-3′;

wherein Nₓ represents a nucleic acid sequence having x consecutive nucleotides, wherein each N is independently chosen from A, G, C or U, and wherein X is an integer between 18 and 35. In some embodiments, X is 20.

In some embodiments, the truncated sgRNA lacks the third stem loop. In some embodiments, the sgRNA comprises artsgRNA13Δloop3. In some embodiments, the sgRNA comprises a nucleic acid sequence as follows:

(SEQ ID NO: 10)
5′GUCGUCUAUAGGACGGCGAGUUUUUCAACGGGUGUGCCCGUUGAGCUU
CAAGAAGUGGCAC-Nₓ-3′
or
(SEQ ID NO: 99)
5′GUCGUCUAUAGGACGGCGAGUUUUUCAACGGGUGUGCCCGUUGAGCUU
CAAAGAAGUGGCAC-Nₓ-3′;

wherein Nₓ represents a nucleic acid sequence having x consecutive nucleotides, wherein each N is independently chosen from A, G, C or U, and wherein X is an integer between 18 and 35. In some embodiments, X is 20.

In some embodiments, the sgRNA comprises a nucleic acid sequence as follows:

(SEQ ID NO: 100)
5′GUCUAAAGGACAGAAUUUUUCAACGGGUGUGCCAAUGGCCACUUUCCA
GGUGGCAAAGCCCGUUGAACUUCUCAAAAAGAACGCUCGCUCAGUGUUCU
GACGUCGGAUCACUGAGCGAGCGAUCUGAGAAGUGGCAC-Nₓ-3′,

wherein Nₓ represents a nucleic acid sequence having x consecutive nucleotides, wherein each N is independently chosen from A, G, C or U, and wherein X is an integer between 18 and 35.

In some embodiments, the sgRNA comprises a nucleic acid sequence as follows:

(SEQ ID NO: 101)
5′GUCUAAAGGACAGAAUUUUUCAACGGGUGUGCCAAUGGCCACUUUCCA
GGUGGCAAAGCCCGUUGAACUUCAAGCGAAGUGGCAC-Nₓ-3′,

wherein Nₓ represents a nucleic acid sequence having x consecutive nucleotides, wherein each N is independently chosen from A, G, C or U, and wherein X is an integer between 18 and 35.

In some embodiments, the truncated sgRNA lacks the fours stem loop. In some embodiments, the truncated sgRNA lacks one or more (e.g., 2 or 3) of the four stem loops.

In some embodiments, the truncated sgRNA is capable of inducing the split portions of Cas12b into forming a functional Cas12b. In some embodiments, the truncated sgRNA is capable of inducing the split portions of Cas12b into forming a functional Cas12b without any inducer.

In some embodiments, the truncated sgRNA is capable of associating with the split portions of Cas12b to form a functional CRISPR complex in the presence of an inducer. Such truncated sgRNA scaffold is useful in combination with inducer-controlled split Cas12b systems described herein, which can reduce off-target editing and allow more precise control over the split Cas12b systems.

### Constructs and Vectors

Also provided herein are split Cas12b constructs, vectors and expression systems encoding one or more components of the CRISPR-Cas systems described herein, such as the split Cas12b polypeptides and/or gRNAs (e.g., sgRNAs).

In some embodiments, there is provided a first vector comprising a nucleic acid sequence encoding a first split Cas12b polypeptide. In some embodiments, there is provided a second vector comprising a nucleic acid sequence encoding a second split Cas12b polypeptide. In some embodiments, there is provided a third vector comprising a nucleic acid sequence encoding the guide RNA (e.g., sgRNA). In some embodiments, the CRISPR-Cas system comprises the first vector, the second vector, and the third vector. In some embodiments, the CRISPR-Cas system comprises the first vector, the second vector, and the guide RNA. In some embodiments, the guide RNA is encoded by the first vector or the second vector. In some embodiments, the CRISPR-Cas system comprises a single vector encoding the first split Cas12b polypeptide, the second split Cas12b polypeptide and the guide RNA.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In general, a suitable vector contains an origin of replication functional in at least one organism, a promoter sequence, convenient restriction endonuclease sites, and one or more selectable markers. The term “vector” should also be construed to include non-plasmid and non-viral compounds, which facilitate transfer of nucleic acid into cells, such as, for example, polylysine compounds, liposomes, and the like.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used.

Extensive efforts have been employed to deliver CRISPR-Cas with adeno-associated viruses (AAVs). AAVs are prevalent and serologically compatible with a large fraction of the human population (Gao, G. et al. Clades of Adeno-associated viruses are widely disseminated in human tissues. Journal of virology 78, 6381-6388 (2004); Boutin, S. et al. Prevalence of serum IgG and neutralizing factors against adeno-associated virus (AAV) types 1, 2, 5, 6, 8, and 9 in the healthy population: implications for gene therapy using AAV vectors. Hum Gene Ther 21, 704-712 (2010)) and are generally not considered to be pathogenic. Furthermore, AAVs allow both programmable tissue-tropism and systemic delivery (Zincarelli, C, Soltys, S., Rengo, G. & Rabinowitz, J. E. Analysis of AAV serotypes 1-9 mediated gene expression and tropism in mice after systemic injection. Mol Ther 16, 1073-1080 (2008)). The preclinical promise of AAV-CRISPR-Cas9 by correcting genetic defects in mice has been described (Ran, F. A. et al. In vivo genome editing using Staphylococcus aureus Cas9. Nature 520, 186-191 (2015); Nelson, C. E. et al. In vivo genome editing improves muscle function in a mouse model of Duchenne muscular dystrophy. Science 351, 403-407 (2016); Tabebordbar, M. et al. In vivo gene editing in dystrophic mouse muscle and muscle stem cells. Science 351, 407-411 (2016); Long, C. et al. Postnatal genome editing partially restores dystrophin expression in a mouse model of muscular dystrophy. Science 351, 400-403 (2016); Yang, Y. et al. A dual AAV system enables the Cas9-mediated correction of a metabolic liver disease in newborn mice. Nature biotechnology (2016); Yin, H. et al. Therapeutic genome editing by combined viral and non-viral delivery of CRISPR system components in vivo. Nature biotechnology (2016)). Any one of the known AAV vectors for delivering Cas9 and other Cas proteins may be used for delivery of the split Cas12b systems of the present application.

Delivery of a CRISPR-Cas system via an AAV vector is limited, in part because the large Cas transgenes leave little space for additional function-conferring elements within current designs (AAV payload limit<4.7 kb). For example, this obstacle is exacerbated with the most widely used, but larger, Streptococcus pyogenes Cas9 (SpCas9, 4.2 kb), which makes packaging of even the minimum functional cassette extremely challenging. The split Cas12b systems of the present application have advantages over other known CRISPR-Cas systems in the art in that the split Cas12b systems have much reduced transgene sizes by splitting a reference Cas12b protein into two or more portions, which facilitate delivery using AAV vectors. In addition, a split Cas12b system frees up space in an AAV vector, which allows delivery of polypeptides comprising additional functional domains fused to the split Cas12b portions, which are useful for transcriptional regulation and other sequence-specific gene modifications.

Methods of introducing vectors into a mammalian cell are known in the art. The vectors can be transferred into a host cell by physical, chemical, or biological methods.

Physical methods for introducing the vector into a host cell include calcium phosphate precipitation, lipofection, particle bombardment, microinjection, electroporation, and the like. Methods for producing cells comprising vectors and/or exogenous nucleic acids are well known in the art. See, for example, Sambrook et al. (2001) Molecular Cloning: A Laboratory Manual, Cold Spring Harbor Laboratory, New York. In some embodiments, the vector is introduced into the cell by electroporation.

Biological methods for introducing the heterologous nucleic acid into a host cell include the use of DNA and RNA vectors. Viral vectors have become the most widely used method for inserting genes into mammalian, e.g., human cells.

Chemical means for introducing the vector into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, the vector(s) or expression system encoding the CRISPR-Cas systems or components thereof comprise one or more selectable or detectable markers that provide a means to isolate or efficiently select cells that contain and/or have been modified by the CRISPR-Cas system, e.g., at an early stage and on a large scale.

In some embodiments, the split Cas12b constructs may encode additional components, such as reporter proteins. In some embodiments, each split Cas12b constructs encodes a florescent protein, such as GFP or RFP. The reporter proteins may be used to assess co-localization and/or dimerization of the split Cas12b polypeptides, e.g., using microscopy. A nucleic acid sequence encoding a split Cas12b polypeptide may be fused to a nucleic acid sequence encoding an additional component using a sequence encoding a self-cleaving peptide, such as a T2A, P2A, E2A or F2A peptide.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)).

Other methods to confirm the presence of the heterologous nucleic acid in a host cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

In some embodiments, the nucleic acid sequences encoding the first split Cas12b polypeptide, the second split Cas 12b polypeptide, and/or the guide RNA are each operably linked to a promoter. In some embodiments, the promoter is an endogenous promoter with respect to a cell that is engineered using the split Cas12b system. For example, the nucleic acid encoding the split Cas12b polypeptides may be knocked-in to the genome of an engineered mammalian cell downstream of an endogenous promoter using any methods known in the art. In some embodiments, the endogenous promoter is a promoter for an abundant protein, such as beta-actin. In some embodiments, the endogenous promoter is an inducible promoter, for example, inducible by an endogenous activation signal of an engineered mammalian cell. In some embodiments, wherein the engineered mammalian cell is a T cell, the promoter is a T cell activation-dependent promoter (such as an IL-2 promoter, an NFAT promoter, or an NFκB promoter).

In some embodiments, the promoter is a heterologous promoter with respect to a cell that is engineered using the split Cas12b system. Varieties of promoters have been explored for gene expression in mammalian cells, and any of the promoters known in the art may be used in the present application. Promoters may be roughly categorized as constitutive promoters or regulated promoters, such as inducible promoters.

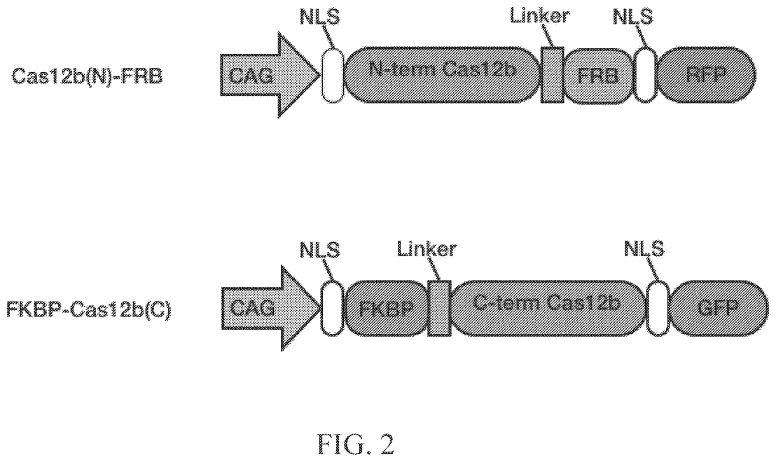

In some embodiments, the nucleic acid sequences encoding the split Cas12b polypeptide(s) and/or the guide RNA are operably linked to a constitutive promoter. Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). In some embodiments, the promoter is a CAG promoter comprising a cytomegalovirus (CMV) early enhancer element, the promoter, the first exon and the first intron of chicken beta-actin gene, and the splice acceptor of the rabbit beta-globin gene. Exemplary engineered split Cas12b constructs are shown in FIGS. 2 and 5-8, in which the CAG promoter is used for expression of the split Cas12b polypeptides.

In some embodiments, the nucleic acid sequences encoding the split Cas12b polypeptide(s) and/or the guide RNA are operably linked to an inducible promoter. Inducible promoters belong to the category of regulated promoters. The inducible promoter can be induced by one or more conditions, such as a physical condition, microenvironment, or the physiological state of a host cell, an inducer (i.e., an inducing agent), or a combination thereof. In some embodiments, the inducing condition is selected from the group consisting of: an inducer, irradiation (such as ionizing radiation, light), temperature (such as heat), redox state, tumor environment, and the activation state of a cell to be engineered by the split Cas12b system. In some embodiments, the promoter is inducible by a small molecule inducer, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most mature system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems.com. Any of the known chemically regulated promoters may be used to drive expression of the split Cas12b polypeptides and/or the guide RNA in the present application.

Also provided are any one of the engineered polypeptides and guide RNAs described herein.

### Methods of Using the CRISPR-Cas Systems

One aspect of the present application provides methods of using the any one of the CRISPR-Cas systems described herein for detecting a target nucleic acid or modifying a nucleic acid in vitro, ex vivo, or in vivo, as well as methods of treatment or diagnosis using the CRISPR-Cas systems. Also provided are use of the CRISPR-Cas systems described herein for detecting or modifying a nucleic acid in a cell, and for treating or diagnosing a disease or condition in a subject; and compositions comprising one or more components of the CRISPR-Cas systems for use in the manufacture of a medicament for detecting or modifying a nucleic acid in a cell, and for treating or diagnosing a disease or condition in a subject.

In some embodiments, the present application provides a method of detecting or modifying a target nucleic acid, comprising contacting the target nucleic acid with any one of the engineered CRISPR-Cas systems described herein, wherein the guide sequence of the guide RNA is complementary to a target sequence of the target nucleic acid, wherein the first polypeptide, the second first polypeptide and the guide RNA associate with each other to bind to the target nucleic acid, thereby detecting or modifying the target nucleic acid. In some embodiments, the target nucleic acid is in a cell. In some embodiments, the method is carried out in vivo. In some embodiments, the method is carried out ex vivo. In some embodiments, the target nucleic acid is not in a cell. In some embodiments, the method is carried out in vitro.

In some embodiments, there is provided a method of detecting a target nucleic acid in a cell, comprising contacting the target nucleic acid with an engineered CRISPR-Cas system comprising: (a) a first polypeptide comprising a N-terminal portion of a reference Cas12b protein, (b) a second polypeptide comprising a C-terminal portion of the reference Cas12b protein, and (c) a guide RNA comprising a guide sequence that is complementary to a target sequence of the target nucleic acid; wherein the reference Cas12b protein comprises from the N-terminus to the C-terminus: WED-I, REC1, WED-II, RuvC-I, BH, REC2, RuvC-II, Nuc-I, RuvC-III and Nuc-II domains; wherein the reference Cas12b protein is enzymatically inactive; wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; and wherein the first polypeptide, the second polypeptide and the guide RNA are capable of associating with each other to form a CRIPSR complex that specifically binds to the target nucleic acid in the cell, thereby detecting the target nucleic acid in the cell. In some embodiments, the first polypeptide comprises a first label, and the second polypeptide comprises a second label, and wherein simultaneous detection (i.e., co-localization) of the first label and the second label allows detection of the target nucleic acid in the cell. In some embodiments, the first label and/or the second label are fluorescent labels, e.g., fluorescent proteins. In some embodiments, the reference Cas12b protein is a Cas12b protein from Alicyclobacillus acidiphilus (AaCas12b), Bh3Cas12b or TcCas12b or a functional derivative thereof. In some embodiments, the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein. In some embodiments, the first polypeptide comprises a first dimerization domain, and the second polypeptide comprises a second dimerization domain. In some embodiments, the method further comprises contacting the target nucleic acid with an inducer. In some embodiments, the method does not comprise contacting the target nucleic acid with an inducer. In some embodiments, the first polypeptide, the second polypeptide and/or the guide RNA are delivered to the cell via one or more viral vectors, e.g., AAV vectors.

The split Cas12b systems described herein can modify a target nucleic acid in a cell in a variety of ways. In some embodiments, the method induces a site-specific cleavage in the target nucleic acid. In some embodiments, the method cleaves a genomic DNA in a cell, such as a bacterial cell, a plant cell, or an animal cell (e.g., a mammalian cell). In some embodiments, the method kills a cell by cleaving a genomic DNA in the cell. In some embodiments, the method cleaves a viral nucleic acid in a cell.

In some embodiments, there is provided a method of cleaving a target nucleic acid in a cell, comprising contacting the target nucleic acid with an engineered CRISPR-Cas system comprising: (a) a first polypeptide comprising a N-terminal portion of a reference Cas12b protein, (b) a second polypeptide comprising a C-terminal portion of the reference Cas12b protein, and (c) a guide RNA comprising a guide sequence that is complementary to a target sequence of the target nucleic acid; wherein the reference Cas12b protein comprises from the N-terminus to the C-terminus: WED-I, REC1, WED-II, RuvC-I, BH, REC2, RuvC-II, Nuc-I, RuvC-III and Nuc-II domains; wherein the reference Cas12b protein is a nuclease that cleaves a single strand or both strands of a duplex nucleic acid; wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; and wherein the first polypeptide, the second polypeptide and the guide RNA are capable of associating with each other to form a CRIPSR complex that specifically binds to and cleaves the target nucleic acid in the cell. In some embodiments, the reference Cas12b protein is a Cas12b protein from Alicyclobacillus acidiphilus (AaCas12b), Bh3Cas12b or TcCas12b or a functional derivative thereof. In some embodiments, the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein. In some embodiments, the first polypeptide comprises a first dimerization domain, and the second polypeptide comprises a second dimerization domain. In some embodiments, the method further comprises contacting the target nucleic acid with an inducer. In some embodiments, the method does not comprise contacting the target nucleic acid with an inducer. In some embodiments, the first polypeptide, the second polypeptide and/or the guide RNA are delivered to the cell via one or more viral vectors, e.g., AAV vectors.

In some embodiments, the method alters (such as increase or decrease) the expression level of the target nucleic acid in the cell. In some embodiments, the method increases the expression level of the target nucleic acid in the cell, e.g., using a split Cas12b system comprising split Cas12b portions based on an enzymatically inactive Cas12b protein fused to transactivation domains. In some embodiments, the method reduces the expression level of the target nucleic acid in the cell, e.g., using a split Cas12b system comprising split Cas12b portions based on an enzymatically inactive Cas12b protein fused to transcription repressor domains. In some embodiments, the method introduces epigenetic modifications to the target nucleic acid in the cell, e.g., using a split Cas12b system comprising split Cas12b portions based on an enzymatically inactive Cas12b protein fused to epigenetic modification domains. The split Cas12b systems described herein may be used to introduce other modifications to the target nucleic acid, depending on the functional domains comprised by the split Cas12b polypeptides.

In some embodiments, there is provided a method of altering the expression level of a target nucleic acid in a cell, comprising contacting the target nucleic acid with an engineered CRISPR-Cas system comprising: (a) a first polypeptide comprising a N-terminal portion of a reference Cas12b protein fused to a first functional domain, (b) a second polypeptide comprising a C-terminal portion of the reference Cas12b protein fused to a second functional domain, and (c) a guide RNA comprising a guide sequence that is complementary to a target sequence of the target nucleic acid; wherein the reference Cas12b protein comprises from the N-terminus to the C-terminus: WED-I, REC1, WED-II, RuvC-I, BH, REC2, RuvC-II, Nuc-I, RuvC-III and Nuc-II domains; wherein the reference Cas12b protein is enzymatically inactive; wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; wherein the first polypeptide, the second polypeptide and the guide RNA are capable of associating with each other to form a CRIPSR complex that specifically binds to the target nucleic acid in the cell; and wherein the first functional domain and the second functional domain alters the expression level of the target nucleic acid. In some embodiments, the first functional domain and the second functional domain are transactivation domains, wherein the method increases the expression level of the target nucleic acid. In some embodiments, the transactivation domains are selected from the group consisting of VP64, p65, HSF1, VP16, MyoD1, HSF1, RTA, SETT/9, and combinations thereof. In some embodiments, the first functional domain and the second functional domain are transcription repressor domains, wherein the method decreases the expression level of the target nucleic acid. In some embodiments, the transcription repressor domains are selected from the group consisting of (KRAB), EnR, NuE, NcoR, SID, and SID4X. In some embodiments, the reference Cas12b protein is a Cas12b protein from Alicyclobacillus acidiphilus (AaCas12b), Bh3Cas12b or TcCas12b or a functional derivative thereof. In some embodiments, the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein. In some embodiments, the first polypeptide comprises a first dimerization domain, and the second polypeptide comprises a second dimerization domain. In some embodiments, the method further comprises contacting the target nucleic acid with an inducer. In some embodiments, the method does not comprise contacting the target nucleic acid with an inducer. In some embodiments, the first polypeptide, the second polypeptide and/or the guide RNA are delivered to the cell via one or more viral vectors, e.g., AAV vectors.

In some embodiments, the method alters a target sequence in the target nucleic acid in the cell. In some embodiments, the method introduces a mutation to the target nucleic acid in the cell. In some embodiments, the method uses one or more endogenous DNA repair pathways, such as Non-homologous end joining (NHEJ) or Homology directed recombination (HDR), in the cell to repair a double-strand break induced in a target DNA as a result of sequence-specific cleavage by the CRISPR complex. Exemplary mutations include, but are not limited to, insertions, deletions, substitutions, and frameshifts. In some embodiments, the method inserts a donor DNA at the target locus. In some embodiments, the insertion of the donor DNA results in introduction of a selection marker or a reporter protein to the cell. In some embodiments, the insertion of the donor DNA results in knock-in of a gene. In some embodiments, the insertion of the donor DNA results in a knockout mutation. In some embodiments, the insertion of the donor DNA results in a substitution mutation, such as a single nucleotide substitution. In some embodiments, the method induces a phenotypic change to the cell.

In some embodiments, there is provided a method of editing a target nucleic acid in a cell comprising contacting the target nucleic acid with an engineered CRISPR-Cas system comprising: (a) a first polypeptide comprising a N-terminal portion of a reference Cas12b protein, (b) a second polypeptide comprising a C-terminal portion of the reference Cas12b protein, and (c) a guide RNA comprising a guide sequence that is complementary to a target sequence in the target nucleic acid; wherein the reference Cas12b protein comprises from the N-terminus to the C-terminus: WED-I, REC1, WED-II, RuvC-I, BH, REC2, RuvC-II, Nuc-I, RuvC-III and Nuc-II domains; wherein the reference Cas12b protein is enzymatically active (e.g., a nuclease that cleaves a single strand or both strands of a duplex nucleic acid); wherein the N-terminal portion of the reference Cas12b protein comprises the WED-I, REC1, and WED-II domains of the reference Cas12b protein; wherein the C-terminal portion of the reference Cas12b protein comprises the RuvC-II, Nuc-I, RuvC-III and Nuc-II domains of the reference Cas12b protein; wherein the RuvC-I, BH, and REC2 domains of the reference Cas12b protein are split between the N-terminal portion of the reference Cas12b protein and the C-terminal portion of the reference Cas12b protein; wherein the first polypeptide, the second polypeptide and the guide RNA are capable of associating with each other to form a CRIPSR complex that specifically binds to and cleaves the target sequence in the target nucleic acid in the cell; and wherein a mutation is introduced at the cleavage site of the target sequence. In some embodiments, the mutation (e.g., indel, substitution or frameshift mutation) is introduced by NHEJ. In some embodiments, the mutation is introduced by HR. In some embodiments, the method further comprises contacting the cell with a donor DNA, wherein the donor DNA is inserted at the cleavage site of the target sequence. In some embodiments, the reference Cas12b protein is a Cas12b protein from Alicyclobacillus acidiphilus (AaCas12b), Bh3Cas12b or TcCas12b or a functional derivative thereof. In some embodiments, the N-terminal portion of the reference Cas12b protein comprises the WED-I, REC1, WED-II, RuvC-I and BH domains of the reference Cas12b protein, and wherein the C-terminal portion of the reference Cas12b protein comprises the REC2, RuvC-II, Nuc-I, RuvC-III and Nuc-II domains of the reference Cas12b protein. In some embodiments, the N-terminal portion of the reference Cas12b protein comprises WED-I, REC1, WED-II, RuvC-I, BH and REC2 domains of the reference Cas12b protein, and wherein the C-terminal portion of the reference Cas12b protein comprises RuvC-II, Nuc-I, RuvC-III and Nuc-II domains of the reference Cas12b protein. In some embodiments, the N-terminal portion of the reference Cas12b protein comprises WED-I, REC1 and WED-II domains of the reference Cas12b protein, and wherein the C-terminal portion of the reference Cas12b protein comprises RuvC-I, BH, REC2, RuvC-II, Nuc-I, RuvC-III and Nuc-II domains of the reference Cas12b protein. In some embodiments, the first polypeptide comprises a first dimerization domain, and the second polypeptide comprises a second dimerization domain. In some embodiments, the method further comprises contacting the target nucleic acid with an inducer. In some embodiments, the method does not comprise contacting the target nucleic acid with an inducer. In some embodiments, the first polypeptide, the second polypeptide, the guide RNA and/or the donor DNA are delivered to the cell via one or more viral vectors, e.g., AAV vectors.

In some embodiments, there is provided a method of assembling a functional Cas12b comprising contacting a target nucleic acid with any one of the engineered split Cas12b systems described herein, wherein the guide sequence of the guide RNA is complementary to a target sequence of the target nucleic acid, wherein the first polypeptide, the second first polypeptide and the guide RNA associate with each other to bind to the target nucleic acid, thereby assembling a functional Cas 12b. In some embodiments, the contacting is in the presence of an inducer. In some embodiments, the contacting is without an inducer.

In some embodiments, there is provided method of inducing nuclease (e.g., cleavage of a single strand or both strands of a duplex nucleic acid) activity of an engineered split Cas12b system, comprising contacting a target nucleic acid with any one of the engineered split Cas12b systems described herein, wherein the guide sequence of the guide RNA is complementary to a target sequence of the target nucleic acid, wherein the first polypeptide, the second first polypeptide and the guide RNA associate with each other to bind to the target nucleic acid, thereby inducing nuclease activity of the engineered Cas 12b system. In some embodiments, the contacting is in the presence of an inducer. In some embodiments, the contacting is without an inducer.

In some embodiments, the engineered CRISPR-Cas system is used a part of a genetic circuit, or for inserting a genetic circuit into the genomic DNA of a cell. The inducer-controlled engineered CRISPR-Cas systems described herein may be especially useful as a component of a genetic circuit. In some embodiments, an inducer-controlled engineered CRISPR-Cas system is used in combination with a truncated sgRNA that reduces auto-induction. Genetic circuits can be useful for gene therapy. Methods and techniques of designing and using genetic circuits are known in the art. Further reference may be made to, for example, Brophy, Jennifer A N, and Christopher A. Voigt. “Principles of genetic circuit design.” Nature methods 11.5 (2014): 508.

The engineered CRISPR-Cas systems described herein are useful for modifying a wide range of target nucleic acids. In some embodiments, the target nucleic acid is in a cell. In some embodiments, the target nucleic acid is a genomic DNA. In some embodiments, the target nucleic acid is an extrachromosomal DNA. In some embodiments, the target nucleic acid is exogenous to a cell. In some embodiments, the target nucleic acid is a viral nucleic acid, such as viral DNA. In some embodiments, the target nucleic acid is a plasmid is a cell. In some embodiments, the target nucleic acid is a horizontally transferred plasmid. In some embodiments, the target nucleic acid is a RNA.

In some embodiments, the target nucleic acid is an isolated nucleic acid, such as an isolated DNA. In some embodiments, the target nucleic acid is present in a cell-free environment. In some embodiments, the target nucleic acid is an isolated vector, such as a plasmid. In some embodiments, the target nucleic acid is an isolated linear DNA fragment.

The methods described herein are applicable for any suitable cell type. In some embodiments, the cell is a bacterium, a yeast cell, a fungal cell, an algal cell, a plant cell, or an animal cell. (e.g., a mammalian cell, such as a human cell). In some embodiments, the cell is a cell isolated from natural sources, such as a tissue biopsy. In some embodiments, the cell is a cell isolated from an in vitro cultured cell line. In some embodiments, the cell is from a primary cell line. In some embodiments, the cell is from an immortalized cell line. In some embodiments, the cell is a genetically engineered cell.

In some embodiments, the cell is an animal cell from an organism selected from the group consisting of cattle, sheep, goat, horse, pig, deer, chicken, duck, goose, rabbit, and fish.

In some embodiments, the cell is a plant cell from an organism selected from the group consisting of maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, and a biomass crop.

In some embodiments, the cell is a mammalian cell. In some embodiments, the cell is a human cell. In some embodiments, the human cell is a human embryonic kidney 293T (HEK293T or 293T) cell or a HeLa cell. In some embodiments, the cell is a human embryonic kidney (HEK293T) cell. In some embodiments, the mammalian the mammalian cell is selected from the group consisting of an immune cell, a hepatic cell, a tumor cell, a stem cell, a zygote, a muscle cell, and a skin cell.

In some embodiments, the cell is an immune cell selected from the group consisting of a cytotoxic T cell, a helper T cell, a natural killer (NK) T cell, an iNK-T cell, an NK-T like cell, a γδT cell, a tumor-infiltrating T cell and a dendritic cell (DC)-activated T cell. In some embodiments, the method produces a modified immune cell, such as a CAR-T cell or a TCR-T cell.

In some embodiments, the cell is an embryonic stem (ES) cell, an induced pluripotent stem (iPS) cell, a progenitor cell of a gamete, a gamete, a zygote, or a cell in an embryo.

The methods described herein can be used to a modify a target cell in vivo, ex vivo or in vitro and, may be conducted in a manner that alters the cell such that once modified the progeny or cell line of the modified cell retains the altered phenotype. The modified cells and progeny may be part of a multi-cellular organism such as a plant or animal with ex vivo or in vivo applications, such as genome editing and gene therapy.

In some embodiments, the method is carried out ex vivo. In some embodiments, the modified cell (e.g., mammalian cell) is propagated ex vivo after introduction of the engineered CRISPR-Cas system into the cell. In some embodiments, the modified cell is cultured to propagate for at least about any of 1 day, 2 days, 3 days, 4 days, 5 days, 6 days, 7 days, 10 days, 12 days, or 14 days. In some embodiments, the modified cell is cultured for no more than about any of 1 day, 2 days, 3 days, 4 days, 5 days, 6 days, 7 days, 10 days, 12 days, or 14 days. In some embodiments, the modified cell is further evaluated or screened to select cells with one or more desirable phenotypes or properties.

In some embodiments, the target sequence is a sequence associated with a disease or condition. Exemplary diseases or conditions include, but are not limited to, cancer, cardiovascular diseases, hereditary diseases, autoimmune diseases, metabolic diseases, neurodegenerative diseases, ocular diseases, bacterial infections and viral infections. In some embodiments, the disease or condition is a genetic disease. In some embodiments, the disease or condition is a monogenetic disease or condition. In some embodiments, the disease or condition is a polygenetic disease or condition.

In some embodiments, the target sequence has a mutation compared to a wildtype sequence. In some embodiments, the target sequence has a single-nucleotide polymorphism (SNP) associated with a disease or condition.

In some embodiments, the donor DNA that is inserted into the target nucleic acid encodes a biological product selected from the group consisting of a reporter protein, an antigen-specific receptor, a therapeutic protein, an antibiotic resistance protein, an RNAi molecule, a cytokine, a kinase, an antigen, an antigen-specific receptor, a cytokine receptor, and a suicide polypeptide. In some embodiments, the donor DNA encodes a therapeutic protein. In some embodiments, the donor DNA encodes a therapeutic protein useful for gene therapy. In some embodiments, the donor DNA encodes a therapeutic antibody. In some embodiments, the donor DNA encodes an engineered receptor, such as a chimeric antigen receptor (CAR), or an engineered TCR. In some embodiments, the donor DNA encodes a therapeutic RNA, such as a small RNA (e.g., siRNA, shRNA, or miRNA), or a long non-coding RNA (lincRNA).

The methods described herein may be used for multiplex gene editing or regulation at two or more (e.g., 2, 3, 4, 5, 6, 8, 10 or more) different target loci. In some embodiments, the method detects or modifies a plurality of target nucleic acids or target nucleic acid sequences. In some embodiments, the method comprises contacting the target nucleic acid with a guide RNA comprises a plurality (e.g., 2, 3, 4, 5, 6, 8, 10 or more) of crRNA sequences, wherein each crRNA comprises a different target sequence.

Also provided are engineered cells comprising a modified target nucleic acid, which are produced using any one of the methods described herein. The engineered cells may be used for cell therapy. Autologous or allogeneic cells may be used to prepare engineered cells using the methods described herein for cell therapy.

The methods described herein may also be used to generate isogenic lines of cells (e.g., mammalian cells) to study genetic variants.

Also provided are engineered non-human animals comprising the engineered cells described herein. In some embodiments, the engineered non-human animals are genome-edited non-human animals. The engineered non-human animals can be used as disease models.

Techniques for producing non-human genome-edited or transgenic animals are well known in the art and include, but are not limited to, pronuclear microinjection, viral infection, and transformation of embryonic stem cells and induced pluripotent stem (iPS) cells. Detailed methods that can be used include, but are not limited to, those described in Sundberg and Ichiki (2006, Genetically Engineered Mice Handbook, CRC Press) and Gibson (2004, A Primer Of Genome Science 2nd ed. Sunderland, Mass.: Sinauer).

The engineered animals may be of any suitable species, including, but not limited to, such as bovids, equids, ovids, canids, cervids, felids, goats, swine, primates as well as less commonly known mammals such as elephants, deer, zebra, or camels.

Further provided are methods of treatment using any one of the methods of modifying a target nucleic acid in a cell described herein, and methods of diagnosis using any one of the methods of detecting a target nucleic acid described herein.

In some embodiments, the present application provides a method of treating a disease or condition associated with a target nucleic acid in cells of an individual, comprising contacting the target nucleic acid with any one of the engineered CRISPR-Cas systems described herein, wherein the guide sequence of the guide RNA is complementary to a target sequence of the target nucleic acid, wherein the first polypeptide, the second first polypeptide and the guide RNA associate with each other to bind to the target nucleic acid to modify the target nucleic acid, thereby the disease or condition is treated. In some embodiments, a mutation (e.g., knockout or knock-in mutation) is introduced to the target nucleic acid. In some embodiments, expression of the target nucleic acid is enhanced. In some embodiments, expression of the target nucleic acid is inhibited.

In some embodiments, the present application provides a method of treating a disease or condition in an individual, comprising administering to the individual an effective amount of any one of the engineered CRISPR-Cas systems described herein, and a donor DNA encoding a therapeutic agent, wherein the guide sequence of the guide RNA is complementary to a target sequence of a target nucleic acid of the individual, wherein the first polypeptide, the second first polypeptide and the guide RNA associate with each other to bind to the target nucleic acid and inserts the donor DNA in the target sequence, thereby the disease or condition is treated.

In some embodiments, the present application provides a method of treating a disease or condition in an individual, comprising administering to the individual an effective amount of engineered cells comprising a modified target nucleic acid, wherein the engineered cells are prepared by contacting the cell with any one of the engineered CRISPR-Cas systems described herein, wherein the guide sequence of the guide RNA is complementary to a target sequence of the target nucleic acid, wherein the first polypeptide, the second first polypeptide and the guide RNA associate with each other to bind to the target nucleic acid to modify the target nucleic acid. In some embodiments, the engineered cells are immune cells.

In some embodiments, the individual is a human being. In some embodiments, the individual is an animal, e.g., a model animal such as a rodent, a pet, or a farm animal. In some embodiments, the individual is a mammal.

In some embodiments, the disease or condition is selected from the group consisting of cancer, cardiovascular diseases, hereditary diseases, autoimmune diseases, metabolic diseases, neurodegenerative diseases, ocular diseases, bacterial infections and viral infections. In some embodiments, the target nucleic acid is PCSK9. In some embodiments, the disease or condition is a cardiovascular disease. In some embodiments, the disease or condition is a coronary artery disease.

In some embodiments, the present application provides a method of reducing cholesterol levels in an individual, comprising administering to the individual an effective amount of any one of the engineered CRISPR-Cas systems described above, wherein the first polypeptide and/or the second polypeptide comprise a transcription repressor, wherein the guide sequence of the guide RNA is complementary to a target sequence of PCSK9, wherein the first polypeptide, the second first polypeptide and the guide RNA associate with each other to bind to the target sequence of PCSK9, thereby repressing the expression of PCSK9 in the individual. In some embodiments, the method treats diabetes in the individual.

### Kits and Articles of Manufacture

Further provided are compositions, kits, unit dosages, and articles of manufacture comprising one or more components of any one of the engineered CRISPR-Cas systems described herein.

In some embodiments, there is provided a kit comprising: (1) a first AAV vector encoding a first split Cas12b polypeptide of any one of the engineered CRISPR-Cas systems described herein; and (2) a second AAV vector encoding a second split Cas12b polypeptide of the engineered CRISPR-Cas system. In some embodiments, the kit further comprises one or more guide RNAs. In some embodiment, the kit further comprises a donor DNA. In some embodiments, the kit further comprises an inducer, such as rapamycin. In some embodiments, the kit further comprises a cell. In some embodiments, the cell is a human embryonic kidney (HEK293T) cell.

The kits may contain one or more additional components, such as containers, reagents, culturing media, cytokines, buffers, antibodies, and the like to allow propagation of an engineered cell. The kits may also contain a device for administration of the composition.

The kit may further comprise instructions for using the engineered CRISPR-Cas system described herein, such as methods of detecting or modifying a target nucleic acid. In some embodiments, the kit comprises instructions for treating or diagnosing a disease or condition. The instructions relating to the use of the components of the kit generally include information as to dosage, dosing schedule, and route of administration for the intended treatment. The containers may be unit doses, bulk packages (e.g., multi-dose packages) or sub-unit doses. For example, kits may be provided that contain sufficient dosages of the composition as disclosed herein to provide effective treatment of an individual for an extended period. Kits may also include multiple unit doses of the composition and instructions for use, packaged in quantities sufficient for storage and use in pharmacies, for example, hospital pharmacies and compounding pharmacies.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual.

Package insert refers to instructions customarily included in commercial packages of therapeutic products that contain information about the indications, usage, dosage, administration, contraindications and/or warnings concerning the use of such therapeutic products.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The examples below are intended to be purely exemplary of the invention and should therefore not be considered to limit the invention in any way. The following examples and detailed description are offered by way of illustration and not by way of limitation.

### Example 1: Development of Exemplary Split Cas12b Systems

This example describes the development of exemplary split Cas12b systems useful for genome editing. FIG. 1 illustrates a schematic of the exemplary split Cas12b systems. Split Cas12b protein constructs based on AaCas12b (SEQ ID NO: 33), Bs3Cas12b (SEQ ID NO: 85) and TcCas12b (SEQ ID NO: 88) were prepared.

### Materials and Methods

### Constructs

DNA manipulations, including DNA preparation, digestion, ligation, amplification, purification, agarose gel electrophoresis, etc. were conducted according to Molecular Cloning: A Laboratory Manual with some modifications. The split AaCas12b coding sequence fragments were cloned from the full-length AaCas12b vector reported in Teng F. et al., Cell Discovery, 4, Article number: 63 (2018) and assembled into expression vectors via homologous recombination in vitro using NEBUILDER® HiFi DNA Assembly Master Mix (NEB).

FIG. 3 shows the splitting schemes of three split AaCas12b systems, where the arrows indicate the three split locations on a full-length AaCas12b protein sequence. Specifically, Split 1 splits at amino acid residue 518 of the AaCas12b protein, or a corresponding position in a homolog or orthologue. Split 2 splits at amino acid residue 658 of the AaCas12b protein, or a corresponding position in a homolog or orthologue. Split 3 splits at amino acid residue 783 of the AaCas12b protein, or a corresponding position in a homolog or orthologue.

Constructs of split AaCas12b proteins with split position 1 (Split 1 AaCas12b, SEQ ID NOs: 1-2), split position 2 (Split 2 AaCas12b, SEQ ID NOs: 3-4), and split position 3 (Split 3 AaCas12b, SEQ ID NOs: 5-6) with no dimerization domains were made. In addition, Split2 Bh3Cas12b proteins (SEQ ID NOs: 83-84) and Split 2 TcCas12b proteins (SEQ ID NOs: 86-87) with no dimerization domains were made.

FIG. 2 shows schematics of a rapamycin-inducible split Cas12b system, in which split Cas12b proteins are fused to inducible dimerization domains. SEQ ID NOs: 11-16 show the amino acid sequences of the various split AaCas12b proteins (Cas12b sequences are bolded and FRB/FKBP sequences are underlined). SEQ ID NOs: 17-22 show the nucleotide sequences of the constructs encoding the various split AaCas12b proteins.

SEQ ID NO: 23 shows the nucleotide sequence encoding an artificial sgRNA13 scaffold (U6 promoter-sgRNA Scaffold-Spacer-Terminator).

Split1 AaCas12b NT1-FRB (N-fragment) (NLS-Cas12b NT1₁₋₅₁₈-FRB-NLS)
SEQ ID NO: 11
PKKKRKVPGMAVKSMKVKLRLDNMPEIRAGLWKLHTEVNAGVRYYTEWLSLL
RQENLYRRSPNGDGEQECYKTAEECKAELLERLRARQVENGHCGPAGSDDELL
QLARQLYELLVPQAIGAKGDAQQIARKFLSPLADKDAVGGLGIAKAGNKPRWV
RMREAGEPGWEEEKAKAEARKSTDRTADVLRALADFGLKPLMRVYTDSDMSS
VQWKPLRKGQAVRTWDRDMFQQAIERMMSWESWNQRVGEAYAKLVEQKSR
FEQKNFVGQEHLVQLVNQLQQDMKEASHGLESKEQTAHYLTGRALRGSDKVF
EKWEKLDPDAPFDLYDTEIKNVQRRNTRRFGSHDLFAKLAEPKYQALWREDAS
FLTRYAVYNSIVRKLNHAKMFATFTLPDATAHPIWTRFDKLGGNLHQYTFLFNE
FGEGRHAIRFQKLLTVEDGVAKEVDDVTVPISMSAQLDDLLPRDPHELVALYFQ
DYGAEQHLAGEFGGAKIQYRRDQLNHLHARRGARDVYLNLSVRVQSQSEARG
ERSRGGSGSSGGSGGSGGSGEMWHEGLEEASRLYFGERNVKGMFEVLEPLHAMME
RGPQTLKETSFNQAYGRDLMEAQEWCRKYMKSGNVKDLTQAWDLYYHVFRRISK
QASPKKKRKV
Split1 AaCas12b CT1-FKBP (C-fragment) (NLS-FKBP-Cas12b CT1₅₁₉₋₁₁₂₉-NLS)
SEQ ID NO: 12
PKKKRKVPGMGVQVETISPGDGRTFPKRGQTCVVHYTGMLEDGKKFDSSRDRNKPF
KFMLGKQEVIRGWEEGVAQMSVGQRAKLTISPDYAYGATGHPGIIPPHATLVFDVEL
LKLEGGSGGSGGGSGGGLKRPPYAAVFRLVGDNHRAFVHFDKLSDYLAEHPDDG
KLGSEGLLSGLRVMSVDLGLRTSASISVFRVARKDELKPNSEGRVPFCFPIEGNE
NLVAVHERSQLLKLPGETESKDLRAIREERQRTLRQLRTQLAYLRLLVRCGSED
VGRRERSWAKLIEQPMDANQMTPDWREAFEDELQKLKSLYGICGDREWTEAV
YESVRRVWRHMGKQVRDWRKDVRSGERPKIRGYQKDVVGGNSIEQIEYLERQ
YKFLKSWSFFGKVSGQVIRAEKGSRFAITLREHIDHAKEDRLKKLADRIIMEAL
GYVYALDDERGKGKWVAKYPPCQLILLEELSEYQFNNDRPPSENNQLMQWSH
RGVFQELLNQAQVHDLLVGTMYAAFSSRFDARTGAPGIRCRRVPARCAREQNP
EPFPWWLNKFVAEHKLDGCPLRADDLIPTGEGEFFVSPFSAEEGDFHQIHADLN
AAQNLQRRLWSDFDISQIRLRCDWGEVDGEPVLIPRTTGKRTADSYGNKVFYT
KTGVTYYERERGKKRRKVFAQEELSEEEAELLVEADEAREKSVVLMRDPSGIIN
RGDWTRQKEFWSMVNQRIEGYLVKQIRSRVRLQESACENTGDIASPKKKRKV
Split2 AaCas12b NT2-FRB (N-fragment) (NLS-Cas12b NT2₁₋₆₅₈-FRB-NLS)
SEQ ID NO: 13
PKKKRKVPGMAVKSMKVKLRLDNMPEIRAGLWKLHTEVNAGVRYYTEWLSLL
RQENLYRRSPNGDGEQECYKTAEECKAELLERLRARQVENGHCGPAGSDDELL
QLARQLYELLVPQAIGAKGDAQQIARKFLSPLADKDAVGGLGIAKAGNKPRWV
RMREAGEPGWEEEKAKAEARKSTDRTADVLRALADFGLKPLMRVYTDSDMSS
VQWKPLRKGQAVRTWDRDMFQQAIERMMSWESWNQRVGEAYAKLVEQKSR
FEQKNFVGQEHLVQLVNQLQQDMKEASHGLESKEQTAHYLTGRALRGSDKVF
EKWEKLDPDAPFDLYDTEIKNVQRRNTRRFGSHDLFAKLAEPKYQALWREDAS
FLTRYAVYNSIVRKLNHAKMFATFTLPDATAHPIWTRFDKLGGNLHQYTFLFNE
FGEGRHAIRFQKLLTVEDGVAKEVDDVTVPISMSAQLDDLLPRDPHELVALYFQ
DYGAEQHLAGEFGGAKIQYRRDQLNHLHARRGARDVYLNLSVRVQSQSEARG
ERRPPYAAVFRLVGDNHRAFVHFDKLSDYLAEHPDDGKLGSEGLLSGLRVMSV
DLGLRTSASISVFRVARKDELKPNSEGRVPFCFPIEGNENLVAVHERSQLLKLPG
ETESKDLRAIREERQRTLRQLRTQLAYLRLLVRCSRGGSGSSGGSGGSGGSGEM
WHEGLEEASRLYFGERNVKGMFEVLEPLHAMMERGPQTLKETSFNQAYGRDLMEA
QEWCRKYMKSGNVKDLTQAWDLYYHVFRRISKQASPKKKRKV
Split2 AaCas12b CT2-FKBP (C-fragment) (NLS-FKBP-Cas12b CT2₆₅₉₋₁₁₂₉-NLS)
SEQ ID NO: 14
PKKKRKVPGMGVQVETISPGDGRTFPKRGQTCVVHYTGMLEDGKKFDSSRDRNKPF
KFMLGKQEVIRGWEEGVAQMSVGQRAKLTISPDYAYGATGHPGIIPPHATLVFDVEL
LKLEGGSGGSGGGSGGGLKGSEDVGRRERSWAKLIEQPMDANQMTPDWREAFE
DELQKLKSLYGICGDREWTEAVYESVRRVWRHMGKQVRDWRKDVRSGERPKI
RGYQKDVVGGNSIEQIEYLERQYKFLKSWSFFGKVSGQVIRAEKGSRFAITLRE
HIDHAKEDRLKKLADRIIMEALGYVYALDDERGKGKWVAKYPPCQLILLEELS
EYQFNNDRPPSENNQLMQWSHRGVFQELLNQAQVHDLLVGTMYAAFSSRFDA
RTGAPGIRCRRVPARCAREQNPEPFPWWLNKFVAEHKLDGCPLRADDLIPTGE
GEFFVSPFSAEEGDFHQIHADLNAAQNLQRRLWSDFDISQIRLRCDWGEVDGEP
VLIPRTTGKRTADSYGNKVFYTKTGVTYYERERGKKRRKVFAQEELSEEEAEL
LVEADEAREKSVVLMRDPSGIINRGDWTRQKEFWSMVNQRIEGYLVKQIRSRV
RLQESACENTGDIASPKKKRKV
Split3 AaCas12b NT3-FRB (N-fragment) (NLS-Cas12b NT3₁₋₇₈₃-FRB-NLS)
SEQ ID NO: 15
PKKKRKVPGMAVKSMKVKLRLDNMPEIRAGLWKLHTEVNAGVRYYTEWLSLL
RQENLYRRSPNGDGEQECYKTAEECKAELLERLRARQVENGHCGPAGSDDELL
QLARQLYELLVPQAIGAKGDAQQIARKFLSPLADKDAVGGLGIAKAGNKPRWV
RMREAGEPGWEEEKAKAEARKSTDRTADVLRALADFGLKPLMRVYTDSDMSS
VQWKPLRKGQAVRTWDRDMFQQAIERMMSWESWNQRVGEAYAKLVEQKSR
FEQKNFVGQEHLVQLVNQLQQDMKEASHGLESKEQTAHYLTGRALRGSDKVF
EKWEKLDPDAPFDLYDTEIKNVQRRNTRRFGSHDLFAKLAEPKYQALWREDAS
FLTRYAVYNSIVRKLNHAKMFATFTLPDATAHPIWTRFDKLGGNLHQYTFLFNE
FGEGRHAIRFQKLLTVEDGVAKEVDDVTVPISMSAQLDDLLPRDPHELVALYFQ
DYGAEQHLAGEFGGAKIQYRRDQLNHLHARRGARDVYLNLSVRVQSQSEARG
ERRPPYAAVFRLVGDNHRAFVHFDKLSDYLAEHPDDGKLGSEGLLSGLRVMSV
DLGLRTSASISVFRVARKDELKPNSEGRVPFCFPIEGNENLVAVHERSQLLKLPG
ETESKDLRAIREERQRTLRQLRTQLAYLRLLVRCGSEDVGRRERSWAKLIEQP
MDANQMTPDWREAFEDELQKLKSLYGICGDREWTEAVYESVRRVWRHMGKQ
VRDWRKDVRSGERPKIRGYQKDVVGGNSIEQIEYLERQYKFLKSWSFFGKVSG
QVSRGGSGSSGGSGGSGGSGEMWHEGLEEASRLYFGERNVKGMFEVLEPLHAMME
RGPQTLKETSFNQAYGRDLMEAQEWCRKYMKSGNVKDLTQAWDLYYHVFRRISK
QASPKKKRKV
Split3 AaCas12b CT3-FKBP (C-fragment) (NLS-FKBP-Cas12b CT3₇₈₄₋₁₁₂₉-NLS)
SEQ ID NO: 16
PKKKRKVPGMGVQVETISPGDGRTFPKRGQTCVVHYTGMLEDGKKFDSSRDRNKPF
KFMLGKQEVIRGWEEGVAQMSVGQRAKLTISPDYAYGATGHPGIIPPHATLVFDVEL
LKLEGGSGGSGGGSGGGLKIRAEKGSRFAITLREHIDHAKEDRLKKLADRIIMEA
LGYVYALDDERGKGKWVAKYPPCQLILLEELSEYQFNNDRPPSENNQLMQWS
HRGVFQELLNQAQVHDLLVGTMYAAFSSRFDARTGAPGIRCRRVPARCAREQN
PEPFPWWLNKFVAEHKLDGCPLRADDLIPTGEGEFFVSPFSAEEGDFHQIHADL
NAAQNLQRRLWSDFDISQIRLRCDWGEVDGEPVLIPRTTGKRTADSYGNKVFYT
KTGVTYYERERGKKRRKVFAQEELSEEEAELLVEADEAREKSVVLMRDPSGIIN
RGDWTRQKEFWSMVNQRIEGYLVKQIRSRVRLQESACENTGDIASPKKKRKV
Split1 AaCas12b NT1-FRB (N-fragment) (NLS-Cas12b NT1₁₋₁₅₅₄-FRB-NLS)
SEQ ID NO: 17
CCCAAGAAGAAGCGCAAGGTGcccgggATGGCCGTGAAGAGCATGAAGGTGAAGCT
GCGCCTGGACAACATGCCCGAGATCCGCGCCGGCCTGTGGAAGCTGCACACCGA
GGTGAACGCCGGCGTGCGCTACTACACCGAGTGGCTGAGCCTGCTGCGCCAGGA
GAACCTGTACCGCCGCAGCCCCAACGGCGACGGCGAGCAGGAGTGCTACAAGAC
CGCCGAGGAGTGCAAGGCCGAGCTGCTGGAGCGCCTGCGCGCCCGCCAGGTGGA
GAACGGCCACTGCGGCCCCGCCGGCAGCGACGACGAGCTGCTGCAGCTGGCCCG
CCAGCTGTACGAGCTGCTGGTGCCCCAGGCCATCGGCGCCAAGGGCGACGCCCA
GCAGATCGCCCGCAAGTTCCTGAGCCCCCTGGCCGACAAGGACGCCGTGGGCGG
CCTGGGCATCGCCAAGGCCGGCAACAAGCCCCGCTGGGTGCGCATGCGCGAGGC
CGGCGAGCCCGGCTGGGAGGAGGAGAAGGCCAAGGCCGAGGCCCGCAAGAGCA
CCGACCGCACCGCCGACGTGCTGCGCGCCCTGGCCGACTTCGGCCTGAAGCCCCT
GATGCGCGTGTACACCGACAGCGACATGAGCAGCGTGCAGTGGAAGCCCCTGCG
CAAGGGCCAGGCCGTGCGCACCTGGGACCGCGACATGTTCCAGCAGGCCATCGA
GCGCATGATGAGCTGGGAGAGCTGGAACCAGCGCGTGGGCGAGGCCTACGCCAA
GCTGGTGGAGCAGAAGAGCCGCTTCGAGCAGAAGAACTTCGTGGGCCAGGAGCA
CCTGGTGCAGCTGGTGAACCAGCTGCAGCAGGACATGAAGGAGGCCAGCCACGG
CCTGGAGAGCAAGGAGCAGACCGCCCACTACCTGACCGGCCGCGCCCTGCGCGG
CAGCGACAAGGTGTTCGAGAAGTGGGAGAAGCTGGACCCCGACGCCCCCTTCGA
CCTGTACGACACCGAGATCAAGAACGTGCAGCGCCGCAACACCCGCCGCTTCGG
CAGCCACGACCTGTTCGCCAAGCTGGCCGAGCCCAAGTACCAGGCCCTGTGGCG
CGAGGACGCCAGCTTCCTGACCCGCTACGCCGTGTACAACAGCATCGTGCGCAAG
CTGAACCACGCCAAGATGTTCGCCACCTTCACCCTGCCCGACGCCACCGCCCACC
CCATCTGGACCCGCTTCGACAAGCTGGGCGGCAACCTGCACCAGTACACCTTCCT
GTTCAACGAGTTCGGCGAGGGCCGCCACGCCATCCGCTTCCAGAAGCTGCTGACC
GTGGAGGACGGCGTGGCCAAGGAGGTGGACGACGTGACCGTGCCCATCAGCATG
AGCGCCCAGCTGGACGACCTGCTGCCCCGCGACCCCCACGAGCTGGTGGCCCTGT
ACTTCCAGGACTACGGCGCCGAGCAGCACCTGGCCGGCGAGTTCGGCGGCGCCA
AGATCCAGTACCGCCGCGACCAGCTGAACCACCTGCACGCCCGCCGCGGCGCCC
GCGACGTGTACCTGAACCTGAGCGTGCGCGTGCAGAGCCAGAGCGAGGCCCGCG
GCGAGCGCtctagaGGCGGCAGCGGCAGCAGCGGCGGCAGCGGCGGCAGCGGCGGC
AGCGGCGAGATGTGGCACGAGGGCCTGGAGGAGGCCAGCCGCCTGTACTTCGGC
GAGCGCAACGTGAAGGGCATGTTCGAGGTGCTGGAGCCCCTGCACGCCATGATGG
AGCGCGGCCCCCAGACCCTGAAGGAGACCAGCTTCAACCAGGCCTACGGCCGCG
ACCTGATGGAGGCCCAGGAGTGGTGCCGCAAGTACATGAAGAGCGGCAACGTGA
AGGACCTGACCCAGGCCTGGGACCTGTACTACCACGTGTTCCGCCGCATCAGCAA
GCAGgctagcCCCAAGAAGAAGCGCAAGGTG
Split1 AaCas12b CT1-FKBP (C-fragment) (NLS-FKBP-Cas12b CT1₁₅₅₅₋₃₃₈₇-NLS)
SEQ ID NO: 18
CCCAAGAAGAAGCGCAAGGTGcccgggATGGGCGTGCAGGTGGAGACCATCAGCC
CCGGCGACGGCCGCACCTTCCCCAAGCGCGGCCAGACCTGCGTGGTGCACTACA
CCGGCATGCTGGAGGACGGCAAGAAGTTCGACAGCAGCCGCGACCGCAACAAG
CCCTTCAAGTTCATGCTGGGCAAGCAGGAGGTGATCCGCGGCTGGGAGGAGGGC
GTGGCCCAGATGAGCGTGGGCCAGCGCGCCAAGCTGACCATCAGCCCCGACTAC
GCCTACGGCGCCACCGGCCACCCCGGCATCATCCCCCCCCACGCCACCCTGGTGT
TCGACGTGGAGCTGCTGAAGCTGGAGGGCGGCAGCGGCGGCAGCGGCGGCGGC
AGCGGCGGCGGCcttaagCGCCCCCCCTACGCCGCCGTGTTCCGCCTGGTGGGCGAC
AACCACCGCGCCTTCGTGCACTTCGACAAGCTGAGCGACTACCTGGCCGAGCAC
CCCGACGACGGCAAGCTGGGCAGCGAGGGCCTGCTGAGCGGCCTGCGCGTGATG
AGCGTGGACCTGGGCCTGCGCACCAGCGCCAGCATCAGCGTGTTCCGCGTGGCC
CGCAAGGACGAGCTGAAGCCCAACAGCGAGGGCCGCGTGCCCTTCTGCTTCCCC
ATCGAGGGCAACGAGAACCTGGTGGCCGTGCACGAGCGCAGCCAGCTGCTGAAG
CTGCCCGGCGAGACCGAGAGCAAGGACCTGCGCGCCATCCGCGAGGAGCGCCAG
CGCACCCTGCGCCAGCTGCGCACCCAGCTGGCCTACCTGCGCCTGCTGGTGCGCT
GCGGCAGCGAGGACGTGGGCCGCCGCGAGCGCAGCTGGGCCAAGCTGATCGAG
CAGCCCATGGACGCCAACCAGATGACCCCCGACTGGCGCGAGGCCTTCGAGGAC
GAGCTGCAGAAGCTGAAGAGCCTGTACGGCATCTGCGGCGACCGCGAGTGGACC
GAGGCCGTGTACGAGAGCGTGCGCCGCGTGTGGCGCCACATGGGCAAGCAGGTG
CGCGACTGGCGCAAGGACGTGCGCAGCGGCGAGCGCCCCAAGATCCGCGGCTAC
CAGAAGGACGTGGTGGGCGGCAACAGCATCGAGCAGATCGAGTACCTGGAGCGC
CAGTACAAGTTCCTGAAGAGCTGGAGCTTCTTCGGCAAGGTGAGCGGCCAGGTG
ATCCGCGCCGAGAAGGGCAGCCGCTTCGCCATCACCCTGCGCGAGCACATCGAC
CACGCCAAGGAGGACCGCCTGAAGAAGCTGGCCGACCGCATCATCATGGAGGCC
CTGGGCTACGTGTACGCCCTGGACGACGAGCGCGGCAAGGGCAAGTGGGTGGCC
AAGTACCCCCCCTGCCAGCTGATCCTGCTGGAGGAGCTGAGCGAGTACCAGTTC
AACAACGACCGCCCCCCCAGCGAGAACAACCAGCTGATGCAGTGGAGCCACCGC
GGCGTGTTCCAGGAGCTGCTGAACCAGGCCCAGGTGCACGACCTGCTGGTGGGC
ACCATGTACGCCGCCTTCAGCAGCCGCTTCGACGCCCGCACCGGCGCCCCCGGCA
TCCGCTGCCGCCGCGTGCCCGCCCGCTGCGCCCGCGAGCAGAACCCCGAGCCCTT
CCCCTGGTGGCTGAACAAGTTCGTGGCCGAGCACAAGCTGGACGGCTGCCCCCT
GCGCGCCGACGACCTGATCCCCACCGGCGAGGGCGAGTTCTTCGTGAGCCCCTTC
AGCGCCGAGGAGGGCGACTTCCACCAGATCCACGCCGACCTGAACGCCGCCCAG
AACCTGCAGCGCCGCCTGTGGAGCGACTTCGACATCAGCCAGATCCGCCTGCGCT
GCGACTGGGGCGAGGTGGACGGCGAGCCCGTGCTGATCCCCCGCACCACCGGCA
AGCGCACCGCCGACAGCTACGGCAACAAGGTGTTCTACACCAAGACCGGCGTGA
CCTACTACGAGCGCGAGCGCGGCAAGAAGCGCCGCAAGGTGTTCGCCCAGGAGG
AGCTGAGCGAGGAGGAGGCCGAGCTGCTGGTGGAGGCCGACGAGGCCCGCGAG
AAGAGCGTGGTGCTGATGCGCGACCCCAGCGGCATCATCAACCGCGGCGACTGG
ACCCGCCAGAAGGAGTTCTGGAGCATGGTGAACCAGCGCATCGAGGGCTACCTG
GTGAAGCAGATCCGCAGCCGCGTGCGCCTGCAGGAGAGCGCCTGCGAGAACACC
GGCGACATCgctagcCCCAAGAAGAAGCGCAAGGTG
Split2 AaCas12b NT2-FRB (N-fragment) (NLS-Cas12b NT2₁₋₁₉₇₄-FRB-NLS)
SEQ ID NO: 19
CCCAAGAAGAAGCGCAAGGTGcccgggATGGCCGTGAAGAGCATGAAGGTGAAGCT
GCGCCTGGACAACATGCCCGAGATCCGCGCCGGCCTGTGGAAGCTGCACACCGA
GGTGAACGCCGGCGTGCGCTACTACACCGAGTGGCTGAGCCTGCTGCGCCAGGA
GAACCTGTACCGCCGCAGCCCCAACGGCGACGGCGAGCAGGAGTGCTACAAGAC
CGCCGAGGAGTGCAAGGCCGAGCTGCTGGAGCGCCTGCGCGCCCGCCAGGTGGA
GAACGGCCACTGCGGCCCCGCCGGCAGCGACGACGAGCTGCTGCAGCTGGCCCG
CCAGCTGTACGAGCTGCTGGTGCCCCAGGCCATCGGCGCCAAGGGCGACGCCCA
GCAGATCGCCCGCAAGTTCCTGAGCCCCCTGGCCGACAAGGACGCCGTGGGCGG
CCTGGGCATCGCCAAGGCCGGCAACAAGCCCCGCTGGGTGCGCATGCGCGAGGC
CGGCGAGCCCGGCTGGGAGGAGGAGAAGGCCAAGGCCGAGGCCCGCAAGAGCA
CCGACCGCACCGCCGACGTGCTGCGCGCCCTGGCCGACTTCGGCCTGAAGCCCCT
GATGCGCGTGTACACCGACAGCGACATGAGCAGCGTGCAGTGGAAGCCCCTGCG
CAAGGGCCAGGCCGTGCGCACCTGGGACCGCGACATGTTCCAGCAGGCCATCGA
GCGCATGATGAGCTGGGAGAGCTGGAACCAGCGCGTGGGCGAGGCCTACGCCAA
GCTGGTGGAGCAGAAGAGCCGCTTCGAGCAGAAGAACTTCGTGGGCCAGGAGCA
CCTGGTGCAGCTGGTGAACCAGCTGCAGCAGGACATGAAGGAGGCCAGCCACGG
CCTGGAGAGCAAGGAGCAGACCGCCCACTACCTGACCGGCCGCGCCCTGCGCGG
CAGCGACAAGGTGTTCGAGAAGTGGGAGAAGCTGGACCCCGACGCCCCCTTCGA
CCTGTACGACACCGAGATCAAGAACGTGCAGCGCCGCAACACCCGCCGCTTCGG
CAGCCACGACCTGTTCGCCAAGCTGGCCGAGCCCAAGTACCAGGCCCTGTGGCG
CGAGGACGCCAGCTTCCTGACCCGCTACGCCGTGTACAACAGCATCGTGCGCAAG
CTGAACCACGCCAAGATGTTCGCCACCTTCACCCTGCCCGACGCCACCGCCCACC
CCATCTGGACCCGCTTCGACAAGCTGGGCGGCAACCTGCACCAGTACACCTTCCT
GTTCAACGAGTTCGGCGAGGGCCGCCACGCCATCCGCTTCCAGAAGCTGCTGACC
GTGGAGGACGGCGTGGCCAAGGAGGTGGACGACGTGACCGTGCCCATCAGCATG
AGCGCCCAGCTGGACGACCTGCTGCCCCGCGACCCCCACGAGCTGGTGGCCCTGT
ACTTCCAGGACTACGGCGCCGAGCAGCACCTGGCCGGCGAGTTCGGCGGCGCCA
AGATCCAGTACCGCCGCGACCAGCTGAACCACCTGCACGCCCGCCGCGGCGCCC
GCGACGTGTACCTGAACCTGAGCGTGCGCGTGCAGAGCCAGAGCGAGGCCCGCG
GCGAGCGCCGCCCCCCCTACGCCGCCGTGTTCCGCCTGGTGGGCGACAACCACCG
CGCCTTCGTGCACTTCGACAAGCTGAGCGACTACCTGGCCGAGCACCCCGACGAC
GGCAAGCTGGGCAGCGAGGGCCTGCTGAGCGGCCTGCGCGTGATGAGCGTGGAC
CTGGGCCTGCGCACCAGCGCCAGCATCAGCGTGTTCCGCGTGGCCCGCAAGGAC
GAGCTGAAGCCCAACAGCGAGGGCCGCGTGCCCTTCTGCTTCCCCATCGAGGGC
AACGAGAACCTGGTGGCCGTGCACGAGCGCAGCCAGCTGCTGAAGCTGCCCGGC
GAGACCGAGAGCAAGGACCTGCGCGCCATCCGCGAGGAGCGCCAGCGCACCCTG
CGCCAGCTGCGCACCCAGCTGGCCTACCTGCGCCTGCTGGTGCGCTGCtctagaGGC
GGCAGCGGCAGCAGCGGCGGCAGCGGCGGCAGCGGCGGCAGCGGCGAGATGTG
GCACGAGGGCCTGGAGGAGGCCAGCCGCCTGTACTTCGGCGAGCGCAACGTGAA
GGGCATGTTCGAGGTGCTGGAGCCCCTGCACGCCATGATGGAGCGCGGCCCCCAG
ACCCTGAAGGAGACCAGCTTCAACCAGGCCTACGGCCGCGACCTGATGGAGGCC
CAGGAGTGGTGCCGCAAGTACATGAAGAGCGGCAACGTGAAGGACCTGACCCAG
GCCTGGGACCTGTACTACCACGTGTTCCGCCGCATCAGCAAGCAGgctagcCCCAAG
AAGAAGCGCAAGGTG
Split2 AaCas12b CT2-FKBP (C-fragment) (NLS-FKBP-Cas12b CT2₁₉₇₅₋₃₃₈₇-NLS)
SEQ ID NO: 20
CCCAAGAAGAAGCGCAAGGTGcccgggATGGGCGTGCAGGTGGAGACCATCAGCC
CCGGCGACGGCCGCACCTTCCCCAAGCGCGGCCAGACCTGCGTGGTGCACTACA
CCGGCATGCTGGAGGACGGCAAGAAGTTCGACAGCAGCCGCGACCGCAACAAG
CCCTTCAAGTTCATGCTGGGCAAGCAGGAGGTGATCCGCGGCTGGGAGGAGGGC
GTGGCCCAGATGAGCGTGGGCCAGCGCGCCAAGCTGACCATCAGCCCCGACTAC
GCCTACGGCGCCACCGGCCACCCCGGCATCATCCCCCCCCACGCCACCCTGGTGT
TCGACGTGGAGCTGCTGAAGCTGGAGGGCGGCAGCGGCGGCAGCGGCGGCGGC
AGCGGCGGCGGCcttaagGGCAGCGAGGACGTGGGCCGCCGCGAGCGCAGCTGGGC
CAAGCTGATCGAGCAGCCCATGGACGCCAACCAGATGACCCCCGACTGGCGCGA
GGCCTTCGAGGACGAGCTGCAGAAGCTGAAGAGCCTGTACGGCATCTGCGGCGA
CCGCGAGTGGACCGAGGCCGTGTACGAGAGCGTGCGCCGCGTGTGGCGCCACAT
GGGCAAGCAGGTGCGCGACTGGCGCAAGGACGTGCGCAGCGGCGAGCGCCCCA
AGATCCGCGGCTACCAGAAGGACGTGGTGGGCGGCAACAGCATCGAGCAGATCG
AGTACCTGGAGCGCCAGTACAAGTTCCTGAAGAGCTGGAGCTTCTTCGGCAAGG
TGAGCGGCCAGGTGATCCGCGCCGAGAAGGGCAGCCGCTTCGCCATCACCCTGC
GCGAGCACATCGACCACGCCAAGGAGGACCGCCTGAAGAAGCTGGCCGACCGC
ATCATCATGGAGGCCCTGGGCTACGTGTACGCCCTGGACGACGAGCGCGGCAAG
GGCAAGTGGGTGGCCAAGTACCCCCCCTGCCAGCTGATCCTGCTGGAGGAGCTG
AGCGAGTACCAGTTCAACAACGACCGCCCCCCCAGCGAGAACAACCAGCTGATG
CAGTGGAGCCACCGCGGCGTGTTCCAGGAGCTGCTGAACCAGGCCCAGGTGCAC
GACCTGCTGGTGGGCACCATGTACGCCGCCTTCAGCAGCCGCTTCGACGCCCGCA
CCGGCGCCCCCGGCATCCGCTGCCGCCGCGTGCCCGCCCGCTGCGCCCGCGAGC
AGAACCCCGAGCCCTTCCCCTGGTGGCTGAACAAGTTCGTGGCCGAGCACAAGC
TGGACGGCTGCCCCCTGCGCGCCGACGACCTGATCCCCACCGGCGAGGGCGAGT
TCTTCGTGAGCCCCTTCAGCGCCGAGGAGGGCGACTTCCACCAGATCCACGCCGA
CCTGAACGCCGCCCAGAACCTGCAGCGCCGCCTGTGGAGCGACTTCGACATCAG
CCAGATCCGCCTGCGCTGCGACTGGGGCGAGGTGGACGGCGAGCCCGTGCTGAT
CCCCCGCACCACCGGCAAGCGCACCGCCGACAGCTACGGCAACAAGGTGTTCTA
CACCAAGACCGGCGTGACCTACTACGAGCGCGAGCGCGGCAAGAAGCGCCGCA
AGGTGTTCGCCCAGGAGGAGCTGAGCGAGGAGGAGGCCGAGCTGCTGGTGGAG
GCCGACGAGGCCCGCGAGAAGAGCGTGGTGCTGATGCGCGACCCCAGCGGCATC
ATCAACCGCGGCGACTGGACCCGCCAGAAGGAGTTCTGGAGCATGGTGAACCAG
CGCATCGAGGGCTACCTGGTGAAGCAGATCCGCAGCCGCGTGCGCCTGCAGGAG
AGCGCCTGCGAGAACACCGGCGACATCgctagcCCCAAGAAGAAGCGCAAGGTG
Split3 AaCas12b NT3-FRB (N-fragment) (NLS-Cas12b NT3₁₋₂₃₅₂-FRB-NLS)
SEQ ID NO: 21
CCCAAGAAGAAGCGCAAGGTGcccgggATGGCCGTGAAGAGCATGAAGGTGAAGCT
GCGCCTGGACAACATGCCCGAGATCCGCGCCGGCCTGTGGAAGCTGCACACCGA
GGTGAACGCCGGCGTGCGCTACTACACCGAGTGGCTGAGCCTGCTGCGCCAGGA
GAACCTGTACCGCCGCAGCCCCAACGGCGACGGCGAGCAGGAGTGCTACAAGAC
CGCCGAGGAGTGCAAGGCCGAGCTGCTGGAGCGCCTGCGCGCCCGCCAGGTGGA
GAACGGCCACTGCGGCCCCGCCGGCAGCGACGACGAGCTGCTGCAGCTGGCCCG
CCAGCTGTACGAGCTGCTGGTGCCCCAGGCCATCGGCGCCAAGGGCGACGCCCA
GCAGATCGCCCGCAAGTTCCTGAGCCCCCTGGCCGACAAGGACGCCGTGGGCGG
CCTGGGCATCGCCAAGGCCGGCAACAAGCCCCGCTGGGTGCGCATGCGCGAGGC
CGGCGAGCCCGGCTGGGAGGAGGAGAAGGCCAAGGCCGAGGCCCGCAAGAGCA
CCGACCGCACCGCCGACGTGCTGCGCGCCCTGGCCGACTTCGGCCTGAAGCCCCT
GATGCGCGTGTACACCGACAGCGACATGAGCAGCGTGCAGTGGAAGCCCCTGCG
CAAGGGCCAGGCCGTGCGCACCTGGGACCGCGACATGTTCCAGCAGGCCATCGA
GCGCATGATGAGCTGGGAGAGCTGGAACCAGCGCGTGGGCGAGGCCTACGCCAA
GCTGGTGGAGCAGAAGAGCCGCTTCGAGCAGAAGAACTTCGTGGGCCAGGAGCA
CCTGGTGCAGCTGGTGAACCAGCTGCAGCAGGACATGAAGGAGGCCAGCCACGG
CCTGGAGAGCAAGGAGCAGACCGCCCACTACCTGACCGGCCGCGCCCTGCGCGG
CAGCGACAAGGTGTTCGAGAAGTGGGAGAAGCTGGACCCCGACGCCCCCTTCGA
CCTGTACGACACCGAGATCAAGAACGTGCAGCGCCGCAACACCCGCCGCTTCGG
CAGCCACGACCTGTTCGCCAAGCTGGCCGAGCCCAAGTACCAGGCCCTGTGGCG
CGAGGACGCCAGCTTCCTGACCCGCTACGCCGTGTACAACAGCATCGTGCGCAAG
CTGAACCACGCCAAGATGTTCGCCACCTTCACCCTGCCCGACGCCACCGCCCACC
CCATCTGGACCCGCTTCGACAAGCTGGGCGGCAACCTGCACCAGTACACCTTCCT
GTTCAACGAGTTCGGCGAGGGCCGCCACGCCATCCGCTTCCAGAAGCTGCTGACC
GTGGAGGACGGCGTGGCCAAGGAGGTGGACGACGTGACCGTGCCCATCAGCATG
AGCGCCCAGCTGGACGACCTGCTGCCCCGCGACCCCCACGAGCTGGTGGCCCTGT
ACTTCCAGGACTACGGCGCCGAGCAGCACCTGGCCGGCGAGTTCGGCGGCGCCA
AGATCCAGTACCGCCGCGACCAGCTGAACCACCTGCACGCCCGCCGCGGCGCCC
GCGACGTGTACCTGAACCTGAGCGTGCGCGTGCAGAGCCAGAGCGAGGCCCGCG
GCGAGCGCCGCCCCCCCTACGCCGCCGTGTTCCGCCTGGTGGGCGACAACCACCG
CGCCTTCGTGCACTTCGACAAGCTGAGCGACTACCTGGCCGAGCACCCCGACGAC
GGCAAGCTGGGCAGCGAGGGCCTGCTGAGCGGCCTGCGCGTGATGAGCGTGGAC
CTGGGCCTGCGCACCAGCGCCAGCATCAGCGTGTTCCGCGTGGCCCGCAAGGAC
GAGCTGAAGCCCAACAGCGAGGGCCGCGTGCCCTTCTGCTTCCCCATCGAGGGC
AACGAGAACCTGGTGGCCGTGCACGAGCGCAGCCAGCTGCTGAAGCTGCCCGGC
GAGACCGAGAGCAAGGACCTGCGCGCCATCCGCGAGGAGCGCCAGCGCACCCTG
CGCCAGCTGCGCACCCAGCTGGCCTACCTGCGCCTGCTGGTGCGCTGCGGCAGCG
AGGACGTGGGCCGCCGCGAGCGCAGCTGGGCCAAGCTGATCGAGCAGCCCATGG
ACGCCAACCAGATGACCCCCGACTGGCGCGAGGCCTTCGAGGACGAGCTGCAGA
AGCTGAAGAGCCTGTACGGCATCTGCGGCGACCGCGAGTGGACCGAGGCCGTGT
ACGAGAGCGTGCGCCGCGTGTGGCGCCACATGGGCAAGCAGGTGCGCGACTGGC
GCAAGGACGTGCGCAGCGGCGAGCGCCCCAAGATCCGCGGCTACCAGAAGGACG
TGGTGGGCGGCAACAGCATCGAGCAGATCGAGTACCTGGAGCGCCAGTACAAGT
TCCTGAAGAGCTGGAGCTTCTTCGGCAAGGTGAGCGGCCAGGTGtctagaGGCGGCA
GCGGCAGCAGCGGCGGCAGCGGCGGCAGCGGCGGCAGCGGCGAGATGTGGCAC
GAGGGCCTGGAGGAGGCCAGCCGCCTGTACTTCGGCGAGCGCAACGTGAAGGGC
ATGTTCGAGGTGCTGGAGCCCCTGCACGCCATGATGGAGCGCGGCCCCCAGACCC
TGAAGGAGACCAGCTTCAACCAGGCCTACGGCCGCGACCTGATGGAGGCCCAGG
AGTGGTGCCGCAAGTACATGAAGAGCGGCAACGTGAAGGACCTGACCCAGGCCT
GGGACCTGTACTACCACGTGTTCCGCCGCATCAGCAAGCAGgctagcCCCAAGAAGA
AGCGCAAGGTG
Split3 AaCas12b CT3-FKBP (C-fragment) (NLS-FKBP-Cas12b CT3₂₃₅₃₋₃₃₈₇-NLS)
SEQ ID NO: 22
CCCAAGAAGAAGCGCAAGGTGcccgggATGGGCGTGCAGGTGGAGACCATCAGCC
CCGGCGACGGCCGCACCTTCCCCAAGCGCGGCCAGACCTGCGTGGTGCACTACA
CCGGCATGCTGGAGGACGGCAAGAAGTTCGACAGCAGCCGCGACCGCAACAAG
CCCTTCAAGTTCATGCTGGGCAAGCAGGAGGTGATCCGCGGCTGGGAGGAGGGC
GTGGCCCAGATGAGCGTGGGCCAGCGCGCCAAGCTGACCATCAGCCCCGACTAC
GCCTACGGCGCCACCGGCCACCCCGGCATCATCCCCCCCCACGCCACCCTGGTGT
TCGACGTGGAGCTGCTGAAGCTGGAGGGCGGCAGCGGCGGCAGCGGCGGCGGC
AGCGGCGGCGGCcttaagATCCGCGCCGAGAAGGGCAGCCGCTTCGCCATCACCCTG
CGCGAGCACATCGACCACGCCAAGGAGGACCGCCTGAAGAAGCTGGCCGACCGC
ATCATCATGGAGGCCCTGGGCTACGTGTACGCCCTGGACGACGAGCGCGGCAAG
GGCAAGTGGGTGGCCAAGTACCCCCCCTGCCAGCTGATCCTGCTGGAGGAGCTG
AGCGAGTACCAGTTCAACAACGACCGCCCCCCCAGCGAGAACAACCAGCTGATG
CAGTGGAGCCACCGCGGCGTGTTCCAGGAGCTGCTGAACCAGGCCCAGGTGCAC
GACCTGCTGGTGGGCACCATGTACGCCGCCTTCAGCAGCCGCTTCGACGCCCGCA
CCGGCGCCCCCGGCATCCGCTGCCGCCGCGTGCCCGCCCGCTGCGCCCGCGAGC
AGAACCCCGAGCCCTTCCCCTGGTGGCTGAACAAGTTCGTGGCCGAGCACAAGC
TGGACGGCTGCCCCCTGCGCGCCGACGACCTGATCCCCACCGGCGAGGGCGAGT
TCTTCGTGAGCCCCTTCAGCGCCGAGGAGGGCGACTTCCACCAGATCCACGCCGA
CCTGAACGCCGCCCAGAACCTGCAGCGCCGCCTGTGGAGCGACTTCGACATCAG
CCAGATCCGCCTGCGCTGCGACTGGGGCGAGGTGGACGGCGAGCCCGTGCTGAT
CCCCCGCACCACCGGCAAGCGCACCGCCGACAGCTACGGCAACAAGGTGTTCTA
CACCAAGACCGGCGTGACCTACTACGAGCGCGAGCGCGGCAAGAAGCGCCGCA
AGGTGTTCGCCCAGGAGGAGCTGAGCGAGGAGGAGGCCGAGCTGCTGGTGGAG
GCCGACGAGGCCCGCGAGAAGAGCGTGGTGCTGATGCGCGACCCCAGCGGCATC
ATCAACCGCGGCGACTGGACCCGCCAGAAGGAGTTCTGGAGCATGGTGAACCAG
CGCATCGAGGGCTACCTGGTGAAGCAGATCCGCAGCCGCGTGCGCCTGCAGGAG
AGCGCCTGCGAGAACACCGGCGACATCgctagcCCCAAGAAGAAGCGCAAGGTG
Artificial sgRNA13 Scaffold (U6 promoter-sgRNA
Scaffold-Spacer-Terminator; scaffold sequence
is bolded and spacer sequence is italicized)
SEQ ID NO: 23
GAGGGCCTATTTCCCATGATTCCTTCATATTTGCATATACGATACAAGGCTGTTA
GAGAGATAATTGGAATTAATTTGACTGTAAACACAAAGATATTAGTACAAAATA
CGTGACGTAGAAAGTAATAATTTCTTGGGTAGTTTGCAGTTTTAAAATTATGTTTT
AAAATGGACTATCATATGCTTACCGTAACTTGAAAGTATTTCGATTTCTTGGCTTT
ATATATCTTGTGGAAAGGACGAAACACCGGGGTCGTCTATAGGACGGCGAGTT
TTTCAACGGGTGTGCCAATGGCCACTTTCCAGGTGGCAAAGCCCGTTGAGC
TTCAAAGAAGTGGCACNNNNNNNNNNNNNNNNNNNNtttttttt

### Guide RNAs

Targeting single guide RNAs (sgRNAs) for cell transfection assay were constructed by ligating annealed oligos into BasI-digested pUC19-U6-gRNA vectors. These vectors were extracted using a TIANPURE™ Midi Plasmid Kit (Tiangen) and quantified using NANODROP™ 2000 (Thermo Fisher). The guide sequences used for sgRNA constructions are shown in Table 1.

### Cell Culture, Transfection and Fluorescence-Activated Cell Sorting (FACS)

Human embryonic kidney cell line HEK293T cells were maintained in Dulbecco's Modified Eagle's Medium (DMEM, Gibco) supplemented with 10% fetal bovine serum (FBS, Gibco) and 100 U/ml Penicillin and 100 μg/ml Streptomycin (Gibco). At 24 hours prior to transfection, the HEK293T cells were placed into 12-well plates at a density of 150,000 cells/well or passaged into 6-well plates at a ratio of 1:4˜1:6, such that the cell density would reach to 60%-70% in 24 hours of incubation. The cells were then transferred to serum-free opti-MEM medium (Gibco) for transfection. The 293T cells were transfected using Lipofectamine LTX and PLUS reagents (Invitrogen) following the manufacturer's recommended protocol. For each well of the 12-well plate, a total of 1 μg plasmids (split Cas12b N-terminus: split Cas12b C-terminus: sgRNA=2: 2: 1) were used. And for each well of the 6-well plate, a total of 2.5 μg plasmids (split Cas12b N-VPH/KRAB: split Cas12b C-VPH/KRAB:sgRNAs=2:2:1) were used. After 4 to 6 hours, the medium was changed into serum-containing DMEM (Gibco). At 12 hours after transfection, rapamycin was added to the medium to reach a concentration of 200 nM. Fresh DMEM having 200 nM rapamycin was used for incubation every 24 hours thereafter. Cells were harvested at 60 hours post transfection for genotyping analysis directly or sorted using the MoFlo XDP (Beckman Coulter).

### Mutation Analysis Using T7 Endonuclease I (T7EI) Assay and DNA Sequencing

Harvested HEK293T at 60h post transfection were lysed by One Step Mouse GenoTyping Kit (Vazyme). Briefly, cells were lysed with 100 uL Buffer L(supplemented with Proteinase K) per well, and incubated at 55° C. for 3 hours, and denatured at 95° C. for 10 min. Genomic region surrounding the split CRISPR-Cas12b target site for each gene of interest was PCR-amplified using 2×Taq Plus Master Mix (Vazyme) using PCR setup (95° C., 5 min; (95° C., 30 s; 62° C., 30 s; 72° C., 30 s)×35cycles; 72° C., 5 min). The PCR primers used were shown in Table 2.

RNA Extraction and Quantitative qRT-PCR Analysis

Total RNA was extracted with TRIzol reagent (Invitrogen, 15596-018) from the harvested cells at 60h post transfection by FACS and the genomic DNA was removed using the RNase-Free DNase I Kit (Promega, M6101). Then, 1 μg RNA was reverse-transcribed into cDNAs using the reverse Transcription System (Promega, A3500) in a total volume of 20 μl and 0.2 μl of the product was used as the template for qRT-PCR using a SYBR Premix Ex Taq kit (TaKaRa, RR420A) on Agilent Stratagene Mx3005P. The setup of the qRT-PCR program was as follows: (95° C., 5 min; 95° C., 30 s; 62° C., 30 s; 72° C., 30 s)×35cycles; 72° C., 5 min. Relative gene expression was analyzed based on the 2^{-ΔΔCt} method with ACTB gene as internal control. All primers are listed in the Table 3.

TABLE 1
Target sequences in the human genome.

| | | SEQ | |
|---|---|---|---|
| | Genomic Sequence | ID | 5′PAM |
| Editing Target | (5′-3′) | NO | (5′-3′) |
| CCR5 Target 1 | TCCTTCTCCTGAACACCTTC | 49 | TTG |
| CCR5 Target 2 | TTTGGCCTGAATAATTGCAG | 50 | TTC |
| Dnmt1 Target 1 | CCCTTCAGCTAAAATAAAGG | 51 | TTT |
| RNF2 Target 1 | TAGTCATGGTGTTCTTCAAC | 52 | TTG |
| VEGFA Target 1 | GCTCTCAAGACCCACAATCC | 53 | TTT |
| PCSK9 Target1 | AATCAGAGAGGATCTTCCGA | 54 | TTT |
| PCSK9 Target2 | CGGCCTCGCCCTCCCCAGAC | 55 | TTT |
| PCSK9 Target3 | GACGCTGTCTGGGGAGGGCG | 56 | TTT |
| HBG Target1 | TTCTTCATCCCTAGCCAGCC | 57 | TTA |
| HBG Target2 | CCTTGTCAAGGCTATTGGTC | 58 | TTG |
| HBG Target3 | GCCAGGGACCGTTTCAGACA | 59 | TTA |
| HBG Target4 | AGACAGATATTTGCATTGAG | 60 | TTC |
| PLK1 Target1 | TGCTTGGCTGCCAGTACCTG | 89 | TTG |
| PLK1 Target4 | ATCGAGACCTCAAGCTGGGC | 90 | TTC |
| PLK1 Target6 | CCTGAATGAAGATCTGGAGG | 91 | TTT |
| PLK1 Target8 | CTCCTCTTGTGCAGCTCCAG | 92 | TTC |
| PLK1 Target11 | GCCGTAGGTAGTATCGGGCC | 93 | TTT |
| PLK1 Target13 | CGGTGCAGGTACTGGCAGCC | 94 | TTT |
| PLK1 Target20 | TCACCTCCAGATCTTCATTC | 95 | TTT |

TABLE 2
Primer sequences used in amplifying the
human genomic regions surrounding
the CRISPR-Cas 12b target sites.

| Editing | Forward Primer | Reverse Primer |
|---|---|---|
| Target | (5′-3′) | (5′-3′) |
| CCR5 | GCAGCTCTCATTTTCCATAC | GATCGGGTGTAAACTGAGC |
| Target 1 | AGT (SEQ ID NO: 61) | TTG (SEQ ID NO: 62) |
| CCR5 | TCTGCTACTCGGGAATCCTA | TGTCACAAGCCCACAGATA |
| Target 2 | AAA (SEQ ID NO: 63) | TTTC (SEQ ID NO: 64) |
| Dnmt1 | CTCCTGCTCGGTGAATTTGG | TAGTTGATAAGCGAACCTC |
| Target 1 | (SEQ ID NO: 65) | ACAC (SEQ ID NO: 66) |
| RNF2 | GGAGCTGTAGGCGATTATAG | TTCTCAAACCCTGGAAAGCA |
| Target 1 | TTGAA (SEQ ID NO: 67) | CTTT (SEQ ID NO: 68) |
| VEGFA | ACTTCCACCCTCTGTCTTAT | CCAGCTTCCTCAAATCTTAT |
| Target 1 | CTC (SEQ ID NO: 69) | GCA (SEQ ID NO: 70) |

TABLE 3
Primer sequences used in qRT-PCR for
quantifying the expression level of
the CRISPR-Cas 12b target sites.

| | Forward Primer | Reverse Primer |
|---|---|---|
| Target | (5′-3′) | (5′-3′) |
| HBG | TGCCCAGCTGAGTGAACTGC | TGCAGTCACCATCTTCTGCC |
| | (SEQ ID NO: 72) | AG (SEQ ID NO: 73) |
| PCSK9 | ATCACCGACTTCAACAGCGTG | TGACAGTTGAGCACACGCAG |
| | (SEQ ID NO: 74) | (SEQ ID NO: 75) |
| ACTB | AACTGGGACGACATGGAGAAA | GAAGGTCTCAAACATGATCT |
| | AT (SEQ ID NO: 76) | GGG (SEQ ID NO: 77) |

10 μL of each PCR product was taken and subjected to re-annealing process to enable heteroduplex formation following the methods in Li, W., et al. (Nat Biotechnol, 2013. 31(8): p. 684-6). After re-annealing, each PCR product was treated with 0.3 μL T7EI enzyme (NEB) and 1/10 volume of NEBuffer2 (NEB) for digestion at 37° C. for 45 minutes. The digested product was analyzed on a 3% TAE-agarose gel.

Indels were quantitated based on relative band intensities (Cong, L., et al., Science, 2013. 339(6121): p. 819-23). The indel frequency was determined according to the following equation: 100×(1−(1−b/(a+b))^{1/2}), where a is the intensity of the undigested DNA and b is the summed intensity of DNA product digested by T7EI. The T7EI assay-identified positive PCR samples were cloned into pEASY-T1 or pEASY-B vectors (Transgen), transformed into competent E. coli cells, and plated on growth medium. After incubation at 37° C. overnight, single colonies were randomly picked and sequenced by Sanger sequencing. Mutations, or lack thereof, were determined by comparing the obtained sequences with wild-type sequences.

### Results

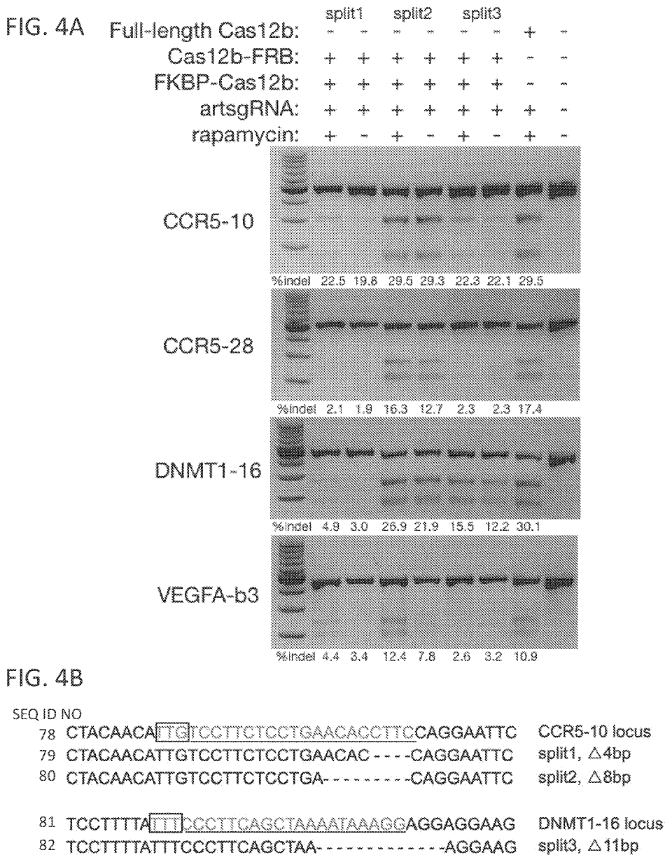

FIG. 4A shows the T7 endonuclease I (T7EI) assay assessing the Insertion-Deletion (InDel) mutations at human target sites induced by the three pairs of split AaCas12b polypeptides in FIG. 3. The human target sites include CCR5-1, CCR5-2, DNMT1, RNF2, and VEGFA. Sanger sequencing results of exemplary mutants using the three split AaCas12b proteins are shown in FIG. 4B. Deleted bases are shown as dashes; PAM sequence is boxed and spacer sequence is underlined.

The results show that the split AaCas12b system induced InDel mutations upon rapamycin treatment at the targeted CCR5-1, CCR5-2, DNMT1, RNF2, and VEGFA locations. The results also suggest that different splitting schemes may have different effects for the split AaCas12b system in inducing InDel mutations. Based on the InDel frequencies (as measured by darkness of the gel bands indicative of digestion and DNA sequencing), Split 2 of the AaCas12b had the highest efficiency across the tested target sites. Additionally, Split 2 showed comparable gene-editing efficiency at multiple target sites without or without induction by rapamycin. This shows that the Split 2 system can be auto-induced.

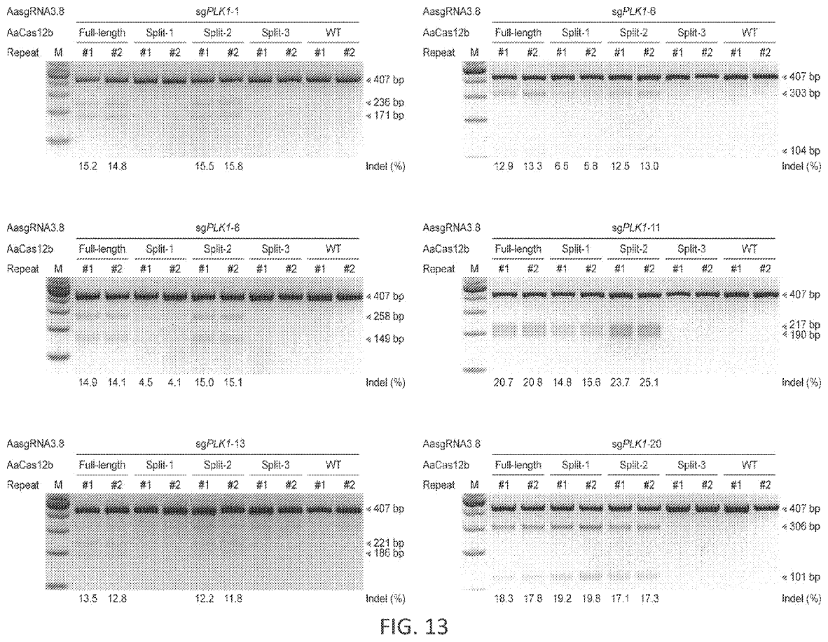

FIG. 13 confirms auto-induced gene-editing activity (i.e., introduction of indel mutations) of Split2 AaCas12b systems without dimerization domains at human all PLK1 target sites tested, and Split1 AaCas12b system also showed auto-induced gene-editing activity at PLK1-6, PLK1-8, PLK1-11 and PLK1-20 target sites. The sgRNA (SEQ ID NO: 101) used in this experiment is a truncated sgRNA based on SEQ ID NO: 100. Notably, the auto-induced gene-editing activity of the Split2 AaCas12b system was comparable to the gene-editing activity of the full-length AaCas12b.

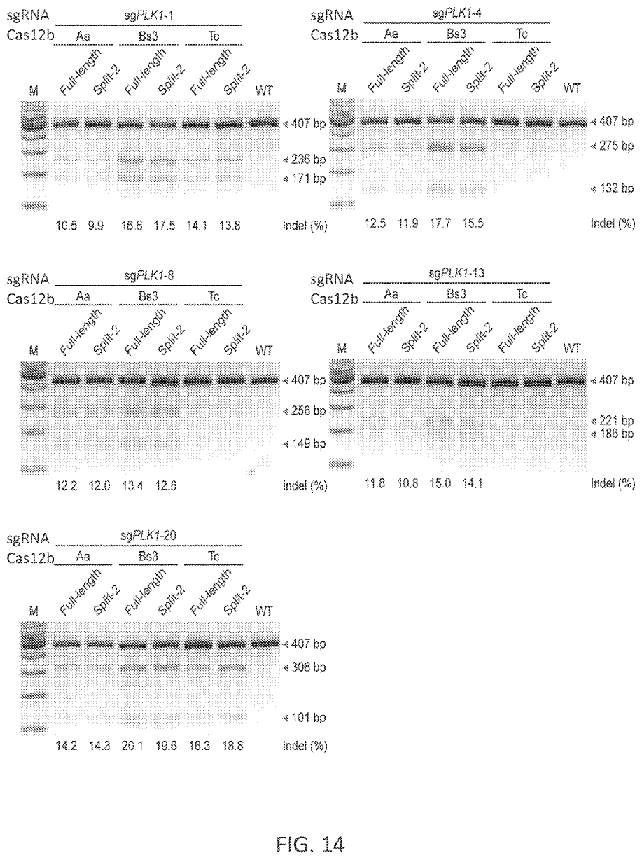

As shown in FIG. 14, similar to the Split2 AaCas12b system with no dimerization domains, the Split2 Bs3Cas12b system with no dimerization domains showed auto-induced gene-editing activity at all PLK-1 target sites tested, while the Split2 TcCas12b system with no dimerization domains showed auto-induced gene-editing activity at PLK1-1 and PLK1-20 target sites. The auto-induced gene-editing activity of the Split2 Cas12b systems at various PLK target sites was comparable to the gene-editing activity of the corresponding full-length Cas12b protein.

Taken together, these data demonstrate that Cas12b can be split into two distinct fragments, which form a functional Cas12 nuclease when brought back together by chemical induction or auto-induction. Thus, this example demonstrates successful development of split AaCas12b systems useful for various fields of basic research and biotechnological applications.

### Example 2: Development of Exemplary Rapamycin-Inducible Split dAaCas12b-Based Gene Activation and Repression Platform

This example describes the development of exemplary rapamycin-inducible split dAaCas12b-based gene activation and repression systems.

### Transcription Activation

FIG. 5 shows a rapamycin-inducible split Cas12b-based gene activation system, comprising a first split Cas12b polypeptide comprising from the N-terminus to the C-terminus: VP64-p65-HSF1, dAaCas12b (catalytically dead Cas12b)N-terminus fragment, and an FRB domain; and a second split Cas12b polypeptide comprising from the N-terminus to the C-terminus: an FKBP domain, dAaCas12b C-terminus fragment, and VP64-p65-HSF1. For dAaCas12b-based transcription activation of endogenous genes, the first split Cas12b polypeptide, the second split Cas12b polypeptide and sgRNAs are co-transfected into human 293T cells. For each gene, different target sgRNAs were constructed and mixed into a pool. All target sites are located within the ˜500 bp upstream of the transcription start site (TSS).

As an example, the Split 2 Cas12b constructs were used to construct a rapamycin-inducible split Cas12b-based gene activation system (also referred herein as “N+C-VPH”), which was used to activate the HBG gene using four target sgRNAs with guide sequences as shown in Table 1. Cells in a well of 6-well plate were transfected with a total of 2.5 μg plasmids (split Cas12b N-VPH: split Cas12b C-VPH:sgRNAs=2: 2: 1). Gene activation was induced by treating the cells with 200 nM rapamycin in serum-containing DMEM (Gibco) at 12h after transfection and GFP⁺/RFP⁺ double positive cells were harvested at 60h post transfection using the MoFlo XDP (Beckman Coulter).

In comparison, full-length dAaCas12b fused to VP64-p65-HSF1 at the N-terminus (i.e., “dAa-VPH”) and the same pool of sgRNAs were co-transfected into human 293T cells. Cells in a well of 6-well plate were transfected with a total of 2.5 μg plasmids (dAa-VPH:sgRNAs=2:1) and GFP⁺ positive cells were harvested at 60h after transfection using the MoFlo XDP (Beckman Coulter). As a negative control, vectors that do not encode N+C-VPH or dAa-VPH were transfected into human 293T cells. qRT-PCR experiments were carried out to determine the transcription level of the HBG gene under each condition.

FIG. 11 shows results of the qRT-PCR experiments that compared gene up-regulation efficiency of rapamycin-inducible split dAaCas12b-VPH (N+C-VPH) and full length dAaCas12b-VPH(dAa-VPH). The results demonstrate that the rapamycin-inducible split dAaCas12b-based gene activation system can up-regulate target gene expression more efficiently than the full-length dAaCas12b-based gene activation system.

### Transcription Repression

FIG. 6 shows a rapamycin-inducible split Cas12b-based gene repression system, comprising a first split Cas12b polypeptide comprising from the N-terminus to the C-terminus: KRAB domain, dAaCas12b N-terminus fragment, and an FRB domain; and a second split Cas12b polypeptide comprising from the N-terminus to the C-terminus: an FKBP domain, dAaCas12b C-terminus fragment, and KRAB domain. For dAaCas12b-based transcription repression of endogenous genes, vectors encoding the first split Cas12b polypeptide, the second split Cas12b polypeptide and sgRNAs were co-transfected into human 293T cells. For each gene, different target sgRNAs were constructed and mixed into a pool. All target sites are located within the ˜500 bp upstream of the transcription start site (TSS).

As an example, the Split 2 Cas12b constructs were used to construct a rapamycin-inducible split Cas12b-based gene repression system (also referred herein as “N+C-KRAB”), which was used to repress the PCSK9 gene using three target sgRNAs with guide sequences as shown in Table 1. Cells in a well of 6-well plate were transfected with a total of 2.5 μg plasmids (split Cas12b N-KRAB: split Cas12b C-KRAB: sgRNAs=2: 2: 1). Gene repression was induced by treating the cells with 200 nM rapamycin at 12h after transfection and GFP⁺/RFP⁺ double positive cells were harvested at 60h post transfection using the MoFLo XDP (Beckman Coulter).

In comparison, full-length dAaCas12b fused to the KRAB domain at the N-terminus (i.e., “dAa-KRAB”) and the same pool of sgRNAs were co-transfected into human 293T cells. Cells in a well of 6-well plate were transfected with a total of 2.5 μg plasmids (dAa-KRAB:sgRNAs=2:1) and GFP⁺ positive cells were harvested at 60h after transfection using the MoFlo XDP (Beckman Coulter). As a negative control, vectors that do not encode N+C-KRAB or dAa-KRAB were transfected into human 293T cells. qRT-PCR experiments were carried out to determine the transcription level of the PCSK9 gene under each condition.

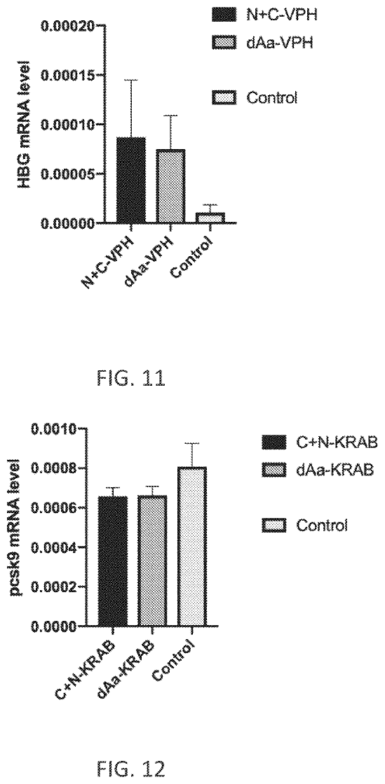

FIG. 12 shows results of the qRT-PCR experiments that compared gene efficiency of rapamycin-inducible split dAaCas12b-KRAB (N+C-KRAB) and full length dAaCas12b-KRAB(dAa-KRAB). The results demonstrate that the rapamycin-inducible split dAaCas12b-based gene repression system can down-regulate target gene expression with a comparable efficiency as the full-length dAaCas12b-based repression system.

### Example 3: Development of Exemplary Auto-Inducing Split dAaCas12b-Based Enhanced Gene Activation and Repression Systems

This example describes the development of exemplary auto-inducing split dAaCas12b-based enhanced gene activation and repression systems.

### Enhanced Transcription Activation

FIG. 7 shows an auto-inducible split Cas12b-based enhanced gene activation system, comprising a first split Cas12b polypeptide comprising from the N-terminus to the C-terminus: VP64-p65-HSF1, dAaCas12b N-terminus fragment, and VP64-p65-HSF1; and a second split Cas12b polypeptide comprising from the N-terminus to the C-terminus: VP64-p65-HSF1, dAaCas12b C-terminus fragment, and VP64-p65-HSF1. For dAaCas12b-based enhanced transcription activation of endogenous genes, the first split Cas12b polypeptide, the second split Cas12b polypeptide and sgRNAs are co-transfected into human 293T cells. For each gene, eight target sgRNAs are constructed and mixed into a pool. All target sites are located within the ˜500 bp upstream of the transcription start site (TSS).

### Enhanced Transcription Repression

FIG. 8 shows an auto-inducible split Cas12b-based enhanced gene activation system, comprising a first split Cas12b polypeptide comprising from the N-terminus to the C-terminus: KRAB domain, dAaCas12b N-terminus fragment, and KRAB domain; and a second split Cas12b polypeptide comprising from the N-terminus to the C-terminus: KRAB domain, dAaCas12b C-terminus fragment, and KRAB domain. For dAaCas12b-based enhanced transcription repression of endogenous genes, the first split Cas12b polypeptide, the second split Cas12b polypeptide and sgRNAs are co-transfected into human 293T cells. For each gene, eight target sgRNAs are constructed and mixed into a pool. All target sites are located within the ˜500 bp upstream of the transcription start site (TSS).

### Example 4: Engineered sgRNA Scaffolds

Truncated sgRNA scaffolds are designed based on artsgRNA13 (FIG. 9) and tested in combination with inducer-controlled split Cas12b polypeptides (e.g., Split 1 and Split 3), and auto-inducing split Cas12b polypeptides (e.g., Split 2 without FKB and FKBP domains) for gene editing efficiency at various target loci in human cells using the T7EI assay described in Example 1. The truncated sgRNA scaffolds lack one or more stem loops from artsgRNA13, including, for example, artsgRNA13Δloop1, artsgRNA13Δloop2, and artsgRNA13Δloop3 (FIG. 10). A truncated sgRNA that is capable of inducing reconstitution of the CRISPR complex using inducer-controlled split Cas12b polypeptides, but not auto-inducing split Cas12b polypeptides is selected to allow tighter control over the split Cas12b system, and to reduce off-target editing events.

### Example 5: In Vivo Gene Repression of PCSK9 Using Split dAaCas12b-Based Enhanced Gene Repression System Reduces Cholesterol Levels in Mice

This example describes in vivo gene repression of PCSK9 using split dAaCas12b-based enhanced gene repression system reduces cholesterol levels in mice. Wildtype adult mice are fed with a high lipid diet to induce high cholesterol levels. AAV vectors encoding a split dAaCas12b-based enhanced gene repression system (e.g., FIG. 8), including sgRNAs targeting PCSK9, are injected intravenously into a first group of mice. In comparison, AAV vectors encoding full-length dAaCas12b-KRAB protein and sgRNAs targeting PCSK9 is injected intravenously into a second group of mice. As a negative control, AAV vectors encoding sgRNAs targeting PCSK9, but no Cas12b proteins or split Cas12b proteins are injected intravenously into a third group of mice. mRNA and protein expression levels of PCSK9 as well as cholesterol levels of each group of mice are determined over time. Mice treated with the split dAaCas12b-based enhanced gene repression system are expected to have reduced cholesterol levels.

1. An engineered Clustered Regularly Interspersed Short Palindromic Repeat (CRISPR)-CRISPR associated (Cas) (CRISPR-Cas) system comprising:
(a) a first polypeptide comprising an N-terminal portion of a reference Cas12b protein,
(b) a second polypeptide comprising a C-terminal portion of the reference Cas12b protein, and
(c) a guide RNA comprising a guide sequence;
wherein the reference Cas12b protein comprises from the N-terminus to the C-terminus: a first WED domain (WED-I), a first REC domain (REC1), a second WED domain (WED-II), a first RuvC domain (RuvC-I), a BH domain, a second REC domain (REC2), a second RuvC domain (RuvC-II), a first Nuc domain (Nuc-I), a third RuvC domain (RuvC-III), and a second Nuc domain (Nuc-II),
wherein the N-terminal portion of the reference Cas12b protein comprises the WED-I, REC1, and WED-II domains of the reference Cas12b protein;
wherein the C-terminal portion of the reference Cas12b protein comprises the RuvC-II, Nuc-I, RuvC-III, and Nuc-II domains of the reference Cas12b protein;
wherein the RuvC-I, BH, and REC2 domains of the reference Cas12b protein are split between the N-terminal portion of the reference Cas12b protein and the C-terminal portion of the reference Cas12b protein; and
wherein the first polypeptide, the second polypeptide and the guide RNA are capable of associating with each other to form a CRIPSR complex that specifically binds to a target nucleic acid comprising a target sequence complementary to the guide sequence.

2. (canceled)

3. The engineered CRISPR-Cas system of claim 1,
(i) wherein the N-terminal portion of the reference Cas12b protein comprises the WED-I, REC1, WED-II, RuvC-I, and BH domains of the reference Cas12b protein, and wherein the C-terminal portion of the reference Cas12b protein comprises the REC2, RuvC-II, Nuc-I, RuvC-III, and Nuc-II domains of the reference Cas12b protein;
(ii) wherein the N-terminal portion of the reference Cas12b protein comprises the WED-I, REC1, WED-II, RuvC-I, BH, and REC2 domains of the reference Cas12b protein, and wherein the C-terminal portion of the reference Cas12b protein comprises the RuvC-II, Nuc-I, RuvC-III, and Nuc-II domains of the reference Cas12b protein;
(iii) wherein the N-terminal portion of the reference Cas12b protein comprises the WED-I, REC1, WED-II, RuvC-I, and BH domains of the reference Cas12b protein, wherein the C-terminal portion of the reference Cas12b protein comprises the RuvC-II, Nuc-I, RuvC-III, and Nuc-II domains of the reference Cas12b protein, and wherein the REC2 domain of the reference Cas12b protein is split between the N-terminal portion of the reference Cas12b protein and the C-terminal portion of the reference Cas12b protein; or
(iv) wherein the N-terminal portion of the reference Cas12b protein comprises the WED-I, REC1, and WED-II domains of the reference Cas12b protein, and wherein the C-terminal portion of the reference Cas12b protein comprises the RuvC-I, BH, REC2, RuvC-II, Nuc-I, RuvC-III, and Nuc-II domains of the reference Cas12b protein.

4-7. (canceled)

8. The engineered CRISPR-Cas system of claim 1, wherein the reference Cas12b protein is a Cas12b protein from Alicyclobacillus acidiphilus (AaCas12b) or a functional derivative thereof.

9. The engineered CRISPR-Cas system of claim 8,
(i) wherein the N-terminal portion of the reference Cas12b protein comprises amino acid residues 1 to 658 of the reference Cas12b protein, and wherein the C-terminal portion of the reference Cas12b protein comprises amino acid residues 659 to 1129 of the reference Cas12b protein;
(ii) wherein the N-terminal portion of the reference Cas12b protein comprises amino acid residues 1 to 783 of the reference Cas12b protein, and wherein the C-terminal portion of the reference Cas12b protein comprises amino acid residues 784 to 1129 of the reference Cas12b protein; or
(iii) wherein the N-terminal portion of the reference Cas12b protein comprises amino acid residues 1 to 518 of the reference Cas12b protein, and wherein the C-terminal portion of the reference Cas12b protein comprises amino acid residues 519 to 1129 of the reference Cas12b protein; and
wherein the amino acid residue numbering is according to SEQ ID NO: 33.

10. The engineered CRISPR-Cas system of claim 9,
(i) wherein the N-terminal portion of the reference Cas12b protein comprises an amino acid sequence having at least about 85% sequence identity to the amino acid sequence of SEQ ID NO: 3, and wherein the C-terminal portion of the reference Cas12b protein comprises an amino acid sequence having at least about 85% sequence identity to the amino acid sequence of SEQ ID NO: 4;
(ii) wherein the N-terminal portion of the reference Cas12b protein comprises an amino acid sequence having at least about 85% sequence identity to the amino acid sequence of SEQ ID NO: 5, and wherein the C-terminal portion of the reference Cas12b protein comprises an amino acid sequence having at least about 85% sequence identity to the amino acid sequence of SEQ ID NO: 6; or
(iii) wherein the N-terminal portion of the reference Cas12b protein comprises an amino acid sequence having at least about 85% sequence identity to the amino acid sequence of SEQ ID NO: 1, and wherein the C-terminal portion of the reference Cas12b protein comprises an amino acid sequence having at least about 85% sequence identity to the amino acid sequence of SEQ ID NO: 2.

11-14. (canceled)

15. The engineered CRISPR-Cas system of claim 1, wherein the reference Cas12b protein is a Cas12b protein from Bacillus sp. V3-13 (Bs3Cas12b) or a functional derivative thereof.

16. The engineered CRISPR-Cas system of claim 15, wherein the N-terminal portion of the reference Cas12b protein comprises amino acid residues 1 to 650 of the reference Cas12b protein, wherein the C-terminal portion of the reference Cas12b protein comprises amino acid residues 651 to 1112 of the reference Cas12b protein, and wherein the amino acid residue numbering is according to SEQ ID NO: 85.

17. The engineered CRISPR-Cas system of claim 16, wherein the N-terminal portion of the reference Cas12b protein comprises an amino acid sequence having at least about 85% sequence identity to the amino acid sequence of SEQ ID NO: 83, and wherein the C-terminal portion of the reference Cas12b protein comprises an amino acid sequence having at least about 85% sequence identity to the amino acid sequence of SEQ ID NO: 84.

18. The engineered CRISPR-Cas system of claim 1, wherein the reference Cas12b protein is a Cas12b protein from Tuberibacillus calidus (TcCas12b) or a functional derivative thereof.

19. The engineered CRISPR-Cas system of claim 18, wherein the N-terminal portion of the reference Cas12b protein comprises amino acid residues 1 to 671 of the reference Cas12b protein, wherein the C-terminal portion of the reference Cas12b protein comprises amino acid residues 672 to 1142 of the reference Cas12b protein, and wherein the amino acid residue numbering is according to SEQ ID NO: 88.

20. The engineered CRISPR-Cas system of claim 19, wherein the N-terminal portion of the reference Cas12b protein comprises an amino acid sequence having at least about 85% sequence identity to the amino acid sequence of SEQ ID NO: 86, and wherein the C-terminal portion of the reference Cas12b protein comprises an amino acid sequence having at least about 85% sequence identity to the amino acid sequence of SEQ ID NO: 87.

21. The engineered CRISPR-Cas system of claim 1, wherein the first polypeptide and the second polypeptide do not comprise dimerization domains.

22. The engineered CRISPR-Cas system of claim 1, wherein the first polypeptide comprises a first dimerization domain, and the second polypeptide comprises a second dimerization domain.

23. The engineered CRISPR-Cas system of claim 22, wherein the first dimerization domain and the second dimerization domain associate with each other in the presence of an inducer.

24-30. (canceled)

31. The engineered CRISPR-Cas system of claim 1, wherein the reference Cas12b protein is enzymatically inactive.

32. The engineered CRISPR-Cas system of claim 31, wherein the reference Cas12b protein comprises one or more mutations selected from the group consisting of D570A, R785A, R911A, and D977A, and wherein the amino acid numbering is according to SEQ ID NO: 33.

33. The engineered CRISPR-Cas system of claim 32, wherein the first polypeptide further comprises a functional domain fused to the N-terminal portion of the reference Cas12b protein, and/or the second polypeptide further comprises a functional domain fused to the C-terminal portion of the reference Cas12b protein.

34. The engineered CRISPR-Cas system of claim 33, wherein the functional domain is selected from the group consisting of a translation initiator domain, a transcription repressor domain, a transactivation domain, an epigenetic modification domain, and a nuclease domain.

35-45. (canceled)

46. A method of modifying a target nucleic acid, comprising: contacting the target nucleic acid with the engineered CRISPR-Cas system of claim 1, wherein the guide sequence of the guide RNA is complementary to a target sequence of the target nucleic acid, wherein the first polypeptide, the second first polypeptide and the guide RNA associate with each other to bind to the target nucleic acid, thereby modifying the target nucleic acid.

47-57. (canceled)

58. A method of treating a disease or condition associated with a target nucleic acid in cells of an individual, comprising modifying the target nucleic acid in the cells of the individual using the method of claim 46, thereby treating the disease or condition.

59-60. (canceled)

61. The engineered CRISPR-Cas system of claim 1, wherein the first polypeptide comprises an amino acid sequence selected from the group consisting of SEQ ID NOs: 11, 13, and 15, and the second polypeptide comprises an amino acid sequence selected from the group consisting of SEQ ID NOs: 12, 14, and 16.

62. An engineered cell comprising a modified target nucleic acid, wherein the target nucleic acid is modified using the method of claim 46.

63. (canceled)
